(19) United States
(12) Patent Application Publication
(10) Pub. No.: US 2014/0228860 A1
(43) Pub. Date: Aug. 14, 2014

(54) Automated Design, Selection, Manufacturing and Implantation of Patient-Adapted and Improved Articular Implants, Designs and Related Guide Tools

(71) Applicants: Daniel Steines, Lexington, MA (US); Philipp Lang, Lexington, MA (US); Wolfgang Fitz, Sherborn, MA (US)

(72) Inventors: Daniel Steines, Lexington, MA (US); Philipp Lang, Lexington, MA (US); Wolfgang Fitz, Sherborn, MA (US)

(73) Assignee: CONFORMIS, INC., Bedford, MA (US)

(21) Appl. No.: 14/236,782

(22) PCT Filed: Aug. 3, 2012
(86) PCT No.: PCT/US12/49472

§ 371 Date: Feb. 3, 2014

Related U.S. Application Data
(60) Provisional application No. 61/514,868, filed on Aug. 3, 2011.

(51) Int. Cl. A61B 19/00

(52) U.S. Cl. CPC A61B 19/50; A61B 19/2203
USPC 606/130

(57) ABSTRACT

Disclosed are automated systems, devices and methods that facilitate the design, selection, manufacturing and/or implantation of improved and/or patient-adapted (e.g., patient-specific and/or patient-engineered) orthopedic implants and guide tools, as well as associated methods, designs and models.

### RELATED APPLICATIONS

This application claims the benefit of: U.S. Ser. No. 61/514,868, entitled “Patient-Adapted and Improved Articular Implants, Designs and Related Guide Tools,” filed Aug. 3, 2011, the disclosure of which is incorporated herein by reference in its entirety.

### TECHNICAL FIELD

This disclosure relates to automated systems, devices and methods that facilitate the design, selection, modification, manufacturing and/or implantation of improved and/or patient-adapted (e.g., patient-specific and/or patient-engineered) orthopedic implants and guide tools, as well as associated methods, designs and models.

### BACKGROUND

Recently, the medical industry has seen a shift towards personalized medicine, including the use of customized and/or patient-specific medical implants. Such advanced implant designs and related devices and methods desirably address the needs of individual patients, which can include creating an optimal fit of implant components with the articular surfaces they replace, improving joint congruity. Better alignment and joint congruity can, for example, lead to greater stability of the joint and better surgical outcomes.

Where a surgical repair and associated implants are intended to be particularized for an individual patient, the patient's unique anatomy can often pose difficulties for fully-automated systems to accommodate. While automated systems excel at repetitive tasks and handling voluminous calculations, the design and/or selection of individualized patient-adapted implant components and/or surgical procedure steps can involve a combination of engineering and intuition that may be problematic for fully automated systems. A need exists, therefore, for methods and system that automate and/or otherwise modify many of the tasks associated with designing, selecting, modifying, manufacturing and/or implanting patient-adapted implant components.

### SUMMARY

Various methods and techniques described herein can enable an automated system to utilize patient anatomical data in modeling and/or otherwise approximating anatomical features of interest for a given patient, and then utilize these models/approximations in various ways to derive and/or select an appropriate surgical plan and/or associated implant components. Such systems and methods can enable and/or facilitate the automation and/or semi-automation of many of the operations inherent with the use of patient-specific and/or patient-adapted joint implant components. In various alternative embodiments, features disclosed herein may also be used to facilitate the selection, adaptation, modification and/or implantation of standard and/or modular implant components and/or systems.

The embodiments described herein include advancements in or that arise out of the area of patient-adapted articular implants tailored to address the needs of individual, single patients. Such patient-adapted articular implants offer advantages over traditional one-size-fits-all approaches and/or a few-sizes-fit-all approach. The advantages include, for example, better fit, more natural movement of the joint, reduction in the amount of bone removed during surgery and a less invasive procedure. Such patient-adapted articular implants can be created from images of the patient's joint. Based on the image data, patient-adapted implant components can be selected and/or designed to include features (e.g., surface contours, curvatures, widths, lengths, thicknesses, and other features) that match existing features in the single, individual patient's joint as well as features that approximate an ideal and/or healthy feature that may not exist in the patient prior to a procedure.

Patient-adapted features can include features that are patient-specific and/or patient-engineered. Patient-specific (or patient-matched) implant component or guide tool features can include features adapted to match one or more of the patient's biological features including structural and/or functional features, for example, one or more biological/anatomical structures, alignments, kinematics, and/or soft tissue features. Patient-engineered (or patient-derived) features of an implant component can be designed, manufactured (e.g., preoperatively designed and manufactured), selected, and/or adapted based, at least partially, on patient-specific data (e.g., information about one or more of the patient's biological features, structural and/or functional) to substantially enhance or improve one or more of the patient's anatomical and/or biological features.

The patient-adapted (e.g., patient-specific and/or patient-engineered) implant components and guide tools described herein can be selected (e.g., from a library), designed (e.g., preoperatively designed including, optionally, manufacturing the components or tools), and/or selected and optionally further adapted (e.g., by selecting a blank component or tool having certain blank features and then optionally altering the blank features to be patient-adapted). Moreover, related methods, such as designs and strategies for resectioning a patient's biological structure also can be selected and/or designed. For example, an implant component bone-facing surface and a resectioning strategy for the corresponding bone can be selected and/or designed together so that an implant component's bone-facing surfaces match the resected surface(s). In addition, one or more guide tools optionally can be selected and/or designed to facilitate the resection cuts that are predetermined in accordance with resectioning strategy and implant component selection and/or design.

In certain embodiments, patient-adapted features of an implant component, guide tool or related method can be achieved by analyzing imaging test data and selecting, adapting and/or designing (e.g., preoperatively selecting from a library and/or designing) an implant component, a guide tool, and/or a procedure having one or more features that have been matched and/or otherwise optimized for the particular patient's biology. The imaging test data can include data from the patient's joint, for example, data generated from an image of the joint such as x-ray imaging, cone beam CT, digital tomosynthesis, and ultrasound, a MRI or CT scan or a PET or SPECT scan, which is processed to generate a varied or corrected version of the joint or of portions of the joint or of surfaces within the joint. Certain embodiments provide methods and devices to create a desired model of a joint or of portions or surfaces of a joint based on data derived from the existing joint. For example, the data can be used to create a model that can be used to analyze the patient's joint and to devise and evaluate a course of corrective action. The data and/or model also can be used to select, adapt and/or design an implant component having one or more patient-adapted features, such as a surface or curvature.

Any one or more steps of the assessment, selection, adaptation and/or design may be partially or fully automated, for example, using a computer-run software program and/or one or more robots. For example, processing of the patient data, the assessment of biological features and/or feature measurements, the assessment of implant component features and/or feature measurements, the optional assessment of resection cut and/or guide tool features and/or feature measurements, the selection and/or design of one or more features of a patient-adapted implant component, the manufacture of the implant components and/or associated guide tools, the preparation of the patient's various anatomical structures and/or the implantation procedure(s) may be partially or wholly automated.

In various embodiments, an automated system can design, adapt and/or select one or more articular implant components that include (a) an outer or external, joint-facing surface and (b) an inner or internal, bone-facing surface. The outer or external, joint-facing surface can include a bearing surface. The inner or internal, bone-facing surface can include one or more patient-engineered bone cuts selected and/or designed using, at least in part, patient-specific data. In certain embodiments, the patient-engineered bone cuts can be selected, adapted and/or designed using patient-specific data to minimize the amount and/or extent of bone resected in one or more corresponding resection cuts. In certain embodiments, the patient-engineered bone cuts can substantially negatively-match one or more of the resection cuts.

In various embodiments, the articular implant component (as well as any subsequent revision components) can be a knee joint implant component, a hip joint implant component, a shoulder joint implant component, or a spinal implant component. For example, the articular implant component can be a knee joint implant component, such as a femoral implant component.

It is to be understood that the features of the various embodiments described herein are not mutually exclusive and may exist in various combinations and permutations.

### BRIEF DESCRIPTION OF THE DRAWINGS

The foregoing and other objects, aspects, features, and advantages of embodiments will become more apparent and may be better understood by referring to the following description, taken in conjunction with the accompanying drawings, in which:

FIG. 2 is a flow chart illustrating an exemplary process that includes selecting and/or designing a patient-adapted implant;

FIG. 9 is a flow chart illustrating an exemplary process for generating a model of a patient's joint (and/or a resection cut, guide tool, and/or implant component);

FIG. 14 displays an image of one embodiment of a user interface for a computer software program for generating models of patient-specific renderings of implant assembly and defects (e.g., osteophyte structures), together with bone models;

FIG. 15 shows an exemplary illustrative flow chart of various high level processes of an exemplary computer software program for generating models of patient-specific renderings of implant assembly and defects (e.g., osteophyte structures), together with bone models;

FIG. 20 depicts a femoral implant component having six bone cuts with the intersect of bone cuts on the inner, bone-facing surface of the implant highlighted;

FIG. 26 is a flow chart illustrating one exemplary process for assessing and selecting and/or designing one or more implant component features and/or feature measurements, and, optionally assessing and selecting and/or designing one or more resection cut features and feature measurements, for a particular patient;

FIG. 27 is an illustrative flow chart showing exemplary steps to assess a joint and select and/or design a suitable replacement implant component;

FIG. 87 is a flow chart illustrating an exemplary process for selecting and/or designing a patient-adapted total knee implant;

FIGS. 188A and 188B are views of a surface outline and a derived model for a patient's femur and tibia;

FIG. 189 depicts an exemplary flowchart of steps in certain embodiments of a deformable segmentation method;

FIGS. 190A through 190O depict various views of an exemplary display interface for one embodiment of a computer program that applies a deformable segmentation method;

FIGS. 194A through 194J depict various steps in one exemplary method of planning an anterior bone cut on a targeted femur of a patient, in preparation for receiving a patient-specific implant;

FIG. 195 depicts a flowchart of steps in various embodiments of a method of designing, selecting, adapting and/or modifying an implant for use in a targeted anatomical site;

FIG. 196 depicts a flowchart of steps in various alternative embodiments of a method of designing, selecting, adapting and/or modifying an implant for use in a targeted anatomical site;

FIG. 197 is a schematic sagittal view of a model of a condyle of a patient's femur including five virtual cuts;

FIG. 198 is a graphical illustration of an exemplary best fit process using a virtual curved derived from the virtual facet cuts of FIG. 197;

FIG. 199 is a schematic sagittal view of a model of a condyle of a patient's femur having virtual cuts; and

FIGS. 200A through 200C are schematic sagittal views of three possible implant designs having five faceted inner surfaces to accommodate bone cuts on the patient's condyle during surgery.

Additional figure descriptions are included in the text below. Unless otherwise denoted in the description for each figure, “M” and “L” in certain figures indicate medial and lateral sides of the view; “A” and “P” in certain figures indicate anterior and posterior sides of the view, and “S” and “I” in certain figures indicate superior and inferior sides of the view.

### DETAILED DESCRIPTION

When a surgeon uses a traditional off-the-shelf implant to replace a patient's joint, for example, a knee joint, hip joint, or shoulder joint, certain features of the implant typically do not match the particular patient's biological features. These mismatches can cause various complications during and after surgery. For example, surgeons may need to extend the surgery time and apply estimates and rules of thumb during surgery to address the mismatches. The hospital typically needs to stock an inventory of standard, off-the-shelf implants as well as instruments and also needs to re-process (e.g., sterilization) these devices before and/or after each surgery. Furthermore, for the patient, complications associated with these mismatches can include pain, discomfort, soft tissue impingement, and an unnatural feeling of the joint during motion, e.g., so-called mid-flexion instability, as well as an altered range of movement and an increased likelihood of implant failure. In order to fit a traditional implant component to a patient's articular bone, surgeons typically remove substantially more of the patient's bone than is necessary to merely clear diseased bone from the site. This removal of substantial portions of the patient's bone frequently diminishes the patient's bone stock to the point that only one subsequent revision implant is possible.

Various embodiments described herein include advancements in or arise out of the area of patient-adapted implants that are tailored to address the needs of individual, single patients. While patient-adapted implants and surgical procedures typically enjoy significant advantages over one-size-fits-all and/or modular system in terms of implant fit and performance, the custom design or individualizing of such systems can often involve significant additional cost to create as compared to mass-produced implants, and often also involve significant delay as compared to simply choosing a pre-stocked implant “off the shelf” for surgery. There exists, therefore, a need in the industry for methods of reducing costs and/or delays in designing, selecting, modifying, manufacturing and implanting patient-adapted implant systems.

### Automated Treatment Systems

Disclosed herein are improvements to various systems, implants, guide tools, and related methods of employing automated systems for designing (e.g., designing and making), selecting, adapting, manufacturing, modifying, manufacturing and/or using and implanting patient-adapted implants and guide tools, that can desirably be applied to any joint including, without limitation, a spine, spinal articulations, an intervertebral disk, a facet joint, a shoulder, an elbow, a wrist, a hand, a finger, a hip, a knee, an ankle, a foot, or a toe joint. Furthermore, various embodiments described herein can apply to methods and procedures, and the design of methods and procedures, for planning and executing resectioning of the patient's anatomy in order to implant the implant components described herein and/or to using the guide tools described herein.

Because each patient's anatomy is unique in various aspects, it can often be difficult or even impossible for a fully-automated system to perform many of the tasks necessary to properly design and/or select an appropriate implant component (or combination of components) for a particular patient's anatomy. Such design and/or selection can involve a combination of engineering and intuition that automated systems, which typically excel at repetitive tasks and voluminous calculations, may find problematic.

In various embodiments, the design, selection, modification, manufacturing and implantation of patient-adapted implant components can include various combinations of the following steps:

(1) Collection of patient image data;

(2) Segmentation, identification and/or classification/conversion of image data into anatomical data;

(3) Modeling of anatomical data;

(4) Design/selection of appropriate bone-facing features of an implant component (and associated surgical preparation of anatomy);

(5) Design/selection of appropriate joint-facing facing features of an implant component;

(6) Design/selection of complete implant component and evaluation of implant design/selection with regards to function and anatomy;

(7) Manufacture implant;

(8) Surgical preparation of the relevant patient anatomy; and

(9) Implantation of the relevant patient-adapted implant component(s).

### Collection of Image Data and Conversion to Anatomical Data

Various embodiments described herein include implant components designed, selected, adapted and/or manufactured using patient-specific data that is collected preoperatively. The patient-specific data can include points, surfaces, and/or landmarks, collectively referred to herein as “reference points.” In certain embodiments, the reference points can be selected and used to derive a varied or altered surface, such as, without limitation, an ideal surface or structure. The patient-specific data can also include functional features, such as patient-specific kinematics and movement patterns (e.g., as described in the “Attaining Acceptable Joint Kinematics” section below).

Sets of reference points can be grouped to form reference structures used to create a model of a joint and/or an implant design. Designed implant surfaces can be derived from single reference points, triangles, polygons, or more complex surfaces, such as parametric or subdivision surfaces, or models of joint material, such as, for example, articular cartilage, subchondral bone, cortical bone, endosteal bone or bone marrow. Various reference points and reference structures can be selected and manipulated to derive a varied or altered surface, such as, without limitation, an ideal surface or structure.

The reference points can be located on or in the joint that will receive the patient-specific implant. For example, the reference points can include weight-bearing surfaces or locations in or on the joint, a cortex in the joint, and/or an endosteal surface of the joint. The reference points also can include surfaces or locations outside of but related to the joint. Specifically, reference points can include surfaces or locations functionally related to the joint. For example, in embodiments directed to the knee joint, reference points can include one or more locations ranging from the hip down to the ankle or foot. The reference points also can include surfaces or locations homologous to the joint receiving the implant. For example, in embodiments directed to a knee, a hip, or a shoulder joint, reference points can include one or more surfaces or locations from the contralateral knee, hip, or shoulder joint.

### Measuring Biological Features

Reference points and/or data for obtaining measurements of a patient's joint, for example, relative-position measurements, length or distance measurements, curvature measurements, surface contour measurements, thickness measurements (in one location or across a surface), volume measurements (filled or empty volume), density measurements, and other measurements, can be obtained using any suitable technique. For example, one dimensional, two-dimensional, and/or three-dimensional measurements can be obtained using data collected from mechanical means, laser devices, electromagnetic or optical tracking systems, molds, materials applied to the articular surface that harden as a negative match of the surface contour, and/or one or more imaging techniques described herein and/or known in the art. Data and measurements can be obtained non-invasively and/or preoperatively. Alternatively, measurements can be obtained intraoperatively, for example, using a probe or other surgical device during surgery.

In certain embodiments, an imaging data collected from the patient, for example, imaging data from one or more of x-ray imaging, digital tomosynthesis, cone beam CT, non-spiral or spiral CT, non-isotropic or isotropic MRI, SPECT, PET, ultrasound, laser imaging, photo-acoustic imaging, is used to qualitatively and/or quantitatively measure one or more of a patient's biological features, one or more of normal cartilage, diseased cartilage, a cartilage defect, an area of denuded cartilage, subchondral bone, cortical bone, endosteal bone, bone marrow, a ligament, a ligament attachment or origin, menisci, labrum, a joint capsule, articular structures, and/or voids or spaces between or within any of these structures. The qualitatively and/or quantitatively measured biological features can include, but are not limited to, one or more of length, width, height, depth and/or thickness; curvature, for example, curvature in two dimensions (e.g., curvature in or projected onto a plane), curvature in three dimensions, and/or a radius or radii of curvature; shape, for example, two-dimensional shape or three-dimensional shape; area, for example, surface area and/or surface contour; perimeter shape; and/or volume of, for example, the patient's cartilage, bone (subchondral bone, cortical bone, endosteal bone, and/or other bone), ligament, and/or voids or spaces between them.

In certain embodiments, measurements of biological features can include any one or more of the illustrative measurements identified in Table 1.

TABLE 1
Exemplary patient-specific measurements of biological features
that can be used in the creation of a model and/or in the
selection and/or design of an implant component

| Anatomical feature | Exemplary measurement |
|---|---|
| Joint-line, joint gap | Location relative to proximal reference point |
| | Location relative to distal reference point |
| | Angle |
| | Gap distance between opposing surfaces in one or |
| | more locations |
| | Location, angle, and/or distance relative to |
| | contralateral joint |
| Soft tissue tension | Joint gap distance |
| and/or balance | Joint gap differential, e.g., medial to lateral |
| Medullary cavity | Shape in one or more dimensions |
| | Shape in one or more locations |
| | Diameter of cavity |
| | Volume of cavity |
| Subchondral bone | Shape in one or more dimensions |
| | Shape in one or more locations |
| | Thickness in one or more dimensions |
| | Thickness in one or more locations |
| | Angle, e.g., resection cut angle |
| Cortical bone | Shape in one or more dimensions |
| | Shape in one or more locations |
| | Thickness in one or more dimensions |
| | Thickness in one or more locations |
| | Angle, e.g., resection cut angle |
| | Portions or all of cortical bone perimeter at an |
| | intended resection level |
| Endosteal bone | Shape in one or more dimensions |
| | Shape in one or more locations |
| | Thickness in one or more dimensions |
| | Thickness in one or more locations |
| | Angle, e.g., resection cut angle |
| Cartilage | Shape in one or more dimensions |
| | Shape in one or more locations |
| | Thickness in one or more dimensions |
| | Thickness in one or more locations |
| | Angle, e.g., resection cut angle |
| Intercondylar notch | Shape in one or more dimensions |
| | Location |
| | Height in one or more locations |
| | Width in one or more locations |
| | Depth in one or more locations |
| | Angle, e.g., resection cut angle |
| Medial condyle | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Lateral condyle | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Trochlea | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Groove location in one or more locations |
| | Trochlear angle, e.g., groove angle in one or more |
| | locations |
| | Slope in one or more locations and/or directions |
| | Angle, e.g., resection cut angle |
| | Portions or all of cortical bone perimeter at an |
| | intended resection level |
| | Resection surface at an intended resection level |
| Medial trochlea | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Central trochlea | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Groove location in one or more locations |
| | Trochlear angle, e.g., groove angle in one or more |
| | locations |
| | Slope in one or more locations and/or directions |
| | Angle, e.g., resection cut angle |
| | Portions or all of cortical bone perimeter at an |
| | intended resection level |
| | Resection surface at an intended resection level |
| Lateral trochlea | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Entire tibia | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | (e.g., medial and/or lateral) |
| | Angle, e.g., resection cut angle |
| | Axes, e.g., A-P and/or M-L axes |
| | Osteophytes |
| | Plateau slope(s), e.g., relative slopes medial and |
| | lateral |
| | Plateau heights(s), e.g., relative heights medial and |
| | lateral |
| | Bearing surface radii, e.g., e.g., relative radii medial |
| | and lateral |
| | Perimeter profile |
| | Portions or all of cortical bone perimeter at an |
| | intended resection level |
| | Resection surface at an intended resection level |
| Medial tibia | 2D and/or 3D shape of a portion or all |
| | Height in one or more locations |
| | Length in one or more locations |
| | Width in one or more locations |
| | Depth in one or more locations |
| | Thickness or height in one or more locations |
| | Curvature in one or more locations |
| | Slope in one or more locations and/or directions |
| | Angle, e.g., resection cut angle |
| | Perimeter profile |
| | Portions or all of cortical bone perimeter at an |
| | intended resection level |
| | Resection surface at an intended resection level |
| Lateral tibia | 2D and/or 3D shape of a portion or all |
| | Height in one or more locations |
| | Length in one or more locations |
| | Width in one or more locations |
| | Depth in one or more locations |
| | Thickness/height in one or more locations |
| | Curvature in one or more locations |
| | Slope in one or more locations and/or directions |
| | Angle, e.g., resection cut angle |
| | Perimeter profile |
| | Portions or all of cortical bone perimeter at an |
| | intended resection level |
| | Resection surface at an intended resection level |
| Entire patella | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Perimeter profile |
| | Angle, e.g., resection cut angle |
| | Portions or all of cortical bone perimeter at an |
| | intended resection level |
| | Resection surface at an intended resection level |
| Medial patella | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Central patella | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Lateral patella | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Femoral head | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Anteversion or retroversion |
| | Portions or all of bone perimeter at an intended |
| | resection level |
| | Resection surface at an intended resection level |
| Femoral neck | 2D and/or 3D shape of a portion or all |
| | Height in one or more locations |
| | Length in one or more locations |
| | Width in one or more locations |
| | Depth in one or more locations |
| | Thickness in one or more locations |
| | Angle in one or more locations |
| | Neck axis in one or more locations |
| | Curvature in one or more locations |
| | Slope in one or more locations and/or directions |
| | Angle, e.g., resection cut angle |
| | Anteversion or retroversion |
| | Leg length |
| | Portions or all of cortical bone perimeter at an |
| | intended resection level |
| | Resection surface at an intended resection level |
| Femoral shaft | 2D and/or 3D shape of a portion or all |
| | Height in one or more locations |
| | Length in one or more locations |
| | Width in one or more locations |
| | Depth in one or more locations |
| | Thickness in one or more locations |
| | Angle in one or more locations |
| | Shaft axis in one or more locations |
| | Curvature in one or more locations |
| | Angle, e.g., resection cut angle |
| | Anteversion or retroversion |
| | Leg length |
| | Portions or all of cortical bone perimeter at an |
| | intended resection level |
| | Resection surface at an intended resection level |
| Acetabulum | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Anteversion or retroversion |
| | Portions or all of cortical bone perimeter at an |
| | intended resection level |
| | Resection surface at an intended resection level |
| Glenoid | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Anteversion or retroversion |
| | Portions or all of cortical bone perimeter at an |
| | intended resection level |
| | Resection surface at an intended resection level |
| Humeral head | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Anteversion or retroversion |
| | Portions or all of cortical bone perimeter at an |
| | intended resection level |
| | Resection surface at an intended resection level |
| Humeral neck | 2D and/or 3D shape of a portion or all |
| | Height in one or more locations |
| | Length in one or more locations |
| | Width in one or more locations |
| | Depth in one or more locations |
| | Thickness in one or more locations |
| | Angle in one or more locations |
| | Neck axis in one or more locations |
| | Curvature in one or more locations |
| | Slope in one or more locations and/or directions |
| | Angle, e.g., resection cut angle |
| | Anteversion or retroversion |
| | Arm length |
| | Portions or all of cortical bone perimeter at an |
| | intended resection level |
| | Resection surface at an intended resection level |
| Humeral shaft | 2D and/or 3D shape of a portion or all |
| | Height in one or more locations |
| | Length in one or more locations |
| | Width in one or more locations |
| | Depth in one or more locations |
| | Thickness in one or more locations |
| | Angle in one or more locations |
| | Shaft axis in one or more locations |
| | Curvature in one or more locations |
| | Angle, e.g., resection cut angle |
| | Anteversion or retroversion |
| | Arm length |
| | Portions or all of cortical bone perimeter at an |
| | intended resection level |
| | Resection surface at an intended resection level |
| Ankle joint | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Elbow | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Wrist | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Hand | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Finger | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle |
| | Portions or all of cortical bone perimeter at an |
| | intended resection level |
| | Resection surface at an intended resection level |
| Spine | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |
| | Portions or all of cortical bone perimeter at an |
| | intended resection level |
| | Resection surface at an intended resection level |
| Spinal facet joint | 2D and/or 3D shape of a portion or all |
| | Height in one or more locations |
| | Length in one or more locations |
| | Width in one or more locations |
| | Depth in one or more locations |
| | Thickness in one or more locations |
| | Curvature in one or more locations |
| | Slope in one or more locations and/or directions |
| | Angle, e.g., resection cut angle |

Depending on the clinical application, a single or any combination or all of the measurements described in Table 1 and/or known in the art can be used. Additional patient-specific measurements and information including structural and functional features that be used in the evaluation can include, for example, joint kinematic measurements, bone density measurements, bone porosity measurements, identification of damaged or deformed tissues or structures, and patient information, such as patient age, weight, gender, ethnicity, activity level, and overall health status.

At various phases, the automated system may assess, compare and/or analyze one or more of the patient-specific measurements (which may include various combinations of such measurements) to information from a database of anatomical features of interest from other patients and/or population groups, which could also include databases of (a) “normalized” patient models, (b) healthy individuals, (c) gender, age, race or activity matched individuals, (d) unhealthy patient individuals, (e) records of previous surgeries of other individuals, and/or (f) any other anatomical database. If desired, the automated system may modify various measurements based on one or more “normalized” patient models or other comparisons or reference to a desired database. For example, a series of patient-specific femoral measurements may be compiled and compared to one or more exemplary femoral or tibial measurements from a library or other database of “normal” femur measurements. Comparisons and analysis thereof may concern, but is not limited to one, more or any combination of the following dimensions: femoral shape, length, width, height, of one or both condyles, intercondylar shapes and dimensions, trochlea shape and dimensions, coronal curvature, sagittal curvature, cortical/cancellous bone volume and/or quality, etc., and a series of recommendations and/or modifications may be accomplished. Any parameter mentioned in the specification and in the various Tables throughout the specification including anatomic, biomechanical and kinematic parameters can be utilized, not only in the knee, but also in the hip, shoulder, ankle, elbow, wrist, spine and other joints. Such analysis may include modification of one or more patient-specific features and/or design criteria for the implant to account for any underlying deformity reflected in the patient-specific measurements.

If desired, the comparison adapt and/or any modified data may be utilized directly to choose or design an appropriate implant to match the compared feature(s) (such as where an pre-existing surgical plan has been previously created for a patient having similar and/or identical relevant anatomical measurements), and a final verification operation may be accomplished to ensure the chosen implant is acceptable and appropriate to the original unmodified patient-specific measurements (i.e., to check that the chosen implant will ultimately “fit” the specific patient anatomy). In alternative embodiments, the various anatomical features may be differently “weighted” during the comparison process (utilizing various formulaic weightings and/or mathematical algorithms), based on their relative importance or other criteria chosen by the automated system, as well as any criteria or assessment guidelines provided by a designer/programmer and/or physician.

In addition to (or if place of) the above-mentioned measurements, it may be desirable to obtain measurements of the targeted joint (as well as surrounding anatomical areas and or other joints of the patient's anatomy) in a load-bearing or otherwise “real-world” condition. Such measurements can potentially yield extremely useful data on the alignment and/or movement of the joint and surrounding structures (as well as the loading conditions of the various joint components)—information which may be difficult to obtain or model from standard imaging techniques (i.e., sitting or lying X-rays, CT-scans and/or MRI imaging). Such load-bearing measurements can include imaging of the patient standing, walking and/or carrying loads of varying sizes and/or weights.

In various embodiments, the patient-specific measurements selected for the evaluation then can be used to select (e.g., from a library), to design, or to select, adapt and/or design an implant component having one or more measurements corresponding to or derived from the one or more of the assessed patient-specific measurements. For example, the implant component can include one or more patient-specific measurements and/or one or more patient-engineered measurements. Optionally, one or more patient-specific models, one or more patient-adapted surgical steps, and/or one or more patient-adapted surgical guide tools also can be selected, adapted and/or designed to include one or more measurements corresponding to or derived from the one or more of these patient-specific measurements.

### Exemplary Measurement and Anatomcial Feature Derivation

Various methods and approaches can be utilized to measure, derive, asses and/or modify desired anatomical features for use in designing a desired joint implant. In a first step of one exemplary method, existing patient information is obtained from patient measurements through the various methods described herein. Such information can include various information regarding a targeted femur, tibia and patella of a targeted knee joint, which in this case includes information regarding the patient's femoral/tibial/patellar shape, length, width, condyle dimensions, features and slopes, angles, e.g., trochlear angles, Q angle, trochlea characteristics, tibial characteristics, tibial tuberosity, medial/lateral slopes, tibial spine height, coronal curvatures, sagittal curvatures and general joint dimensions, as well as any number of biomechanical or kinematic parameters as described in the foregoing sections and Tables and as known in the art. The information can also include anatomical and biomechanical axes, angle and other information from the patient's opposing joint and well as information regarding adjacent joint structures (i.e., hip and/or ankle information) from the treated leg or the opposing leg or both. Additional information collected can include body weight, race, gender, activity level, health conditions, other disease or medical conditions, etc.

If desired, weighting parameters may be assigned to various measurements or series of measurements (or other collected or derived information), as well as to one or more joint surfaces, including opposing joint surfaces. Modifications to the measured parameters may be performed, as previously described.

Next, utilizing various of the collected, modified and/or derived patient-specific information (as well as any optional weighting parameters), the automated system can attempt to identify one or more “matching subjects” from one or more reference databases, comparing features from the matching subject to the patient-specific information, and optionally creating a comparison or “weighting score” to evaluate and display the results of the various comparisons (relative to individual feature comparisons and/or an overall composite score for the comparison of each subject). The databases can comprise information from various sources, including cadaveric data, imaging, biomechanical or kinematic data, historic data and/or data regarding previous knee implant cases from various manufacturers, including ConforMlS-specific case data. Such data can be specific to gender, age, weight, health, size, etc., or can be selected based on weighting (as previously described) or other criteria.

Next, the automated system selects one or more anatomic shapes or features from one or more matching subjects to create one or more “derived anatomic matches” and/or to modify the patient-specific data. The “derived anatomic matches” may comprise the features from one or more subjects, or may comprise a composite anatomy derived from such shapes and/or subjects (which may also be identified and/or derived utilizing a weighting score, if desired). In addition, or if place of, this step, the method may utilize the matching subject data to filter, normalize and/or “smooth” the patient-specific data, which can desirably correct or normalize the patient-specific data and potentially correct the patient-specific data for inherent deformities like osteophytes, axis deformity and/or cartilage degradation.

The derived anatomic matches and/or modified patient-specific data (either alone or in combination with the original patient-specific data) can be utilized to derive, design and/or choose an appropriate implant design and/or placement (and associated surgical procedures and tools) to treat the joint in a desired manner, if one or more useable matches is available.

### Segmentation of Data

In certain embodiments, individual images of a patient's biological structure can be segmented individually and then, in a later step, the segmentation data from each image can be combined. The images that are segmented individually can be one of a series of images, for example, a series of coronal tomographic slices (e.g., front to back) and/or a series of sagittal tomographic slices (e.g., side to side) and/or a series of axial tomographic slices (e.g., top to bottom) of the patient's joint. Segmenting each image individually can create noise in the combined segmented data. As an illustrative example, in an independent segmentation process, an alteration in the segmentation of a single image does not alter the segmentation in contiguous images in a series. Accordingly, an individual image can be segmented to show data that appears discontinuous with data from contiguous images. To address this issue, certain embodiments include a method for generating a model from a collection of images, for example, simultaneously, rather than from individually segmented images. One such method is referred to as deformable segmentation.

In the deformable segmentation method, a template model having a surface data representation, such as for example a parametric surface, a subdivision surface or a meshed surface, is deformed to fit a collection of multiple images. By fitting the template model to a collection of images, alterations to one location in the template model can be carried across the model and, therefore, connect information corresponding to various images in the collection, thus preserving continuity and smoothness of the surface model. For example, in certain embodiments, a template model can include a parametric surface that includes multiple patches or sections. During deformation, the patches can maintain a set of properties, such as continuity, curvature, and/or other properties within each patch and/or across patch boundaries with neighboring patches. These properties also can be reinforced during deformation so that the integrity of the model is maintained.

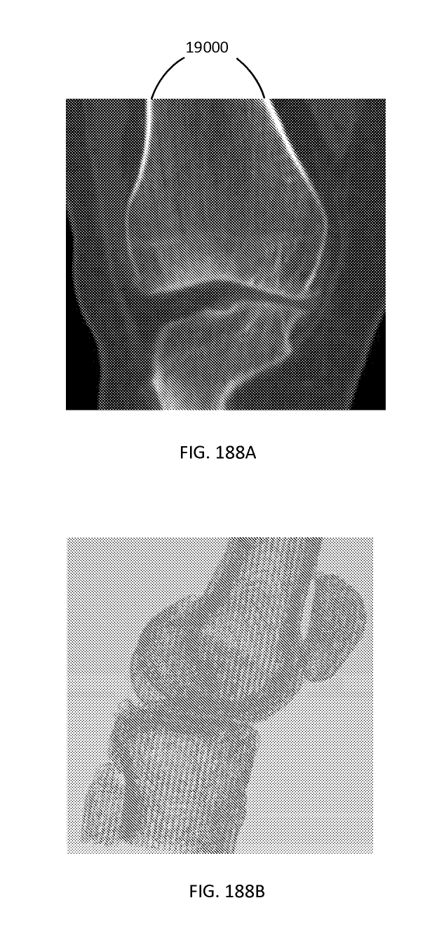

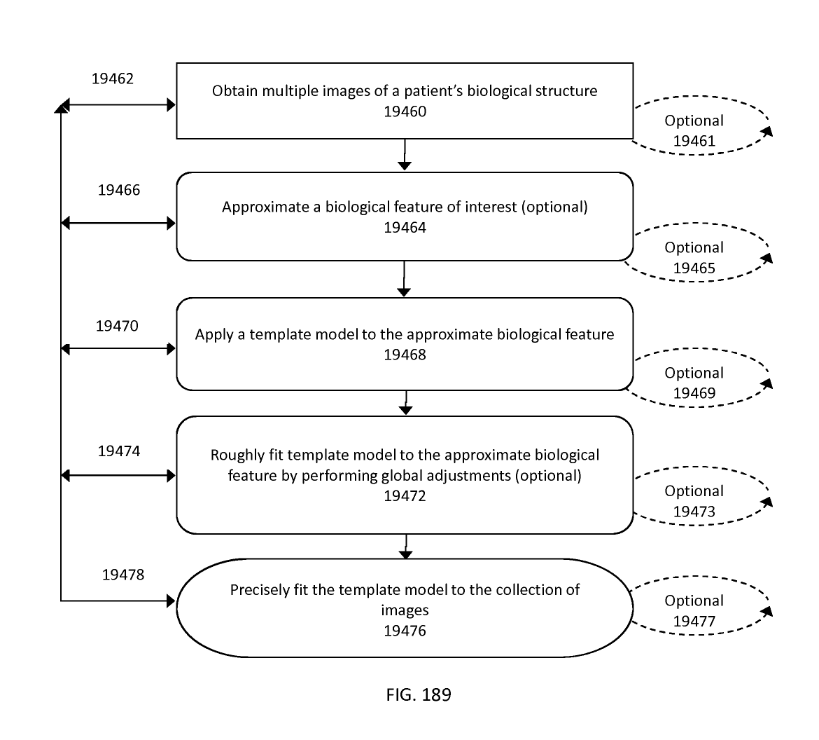

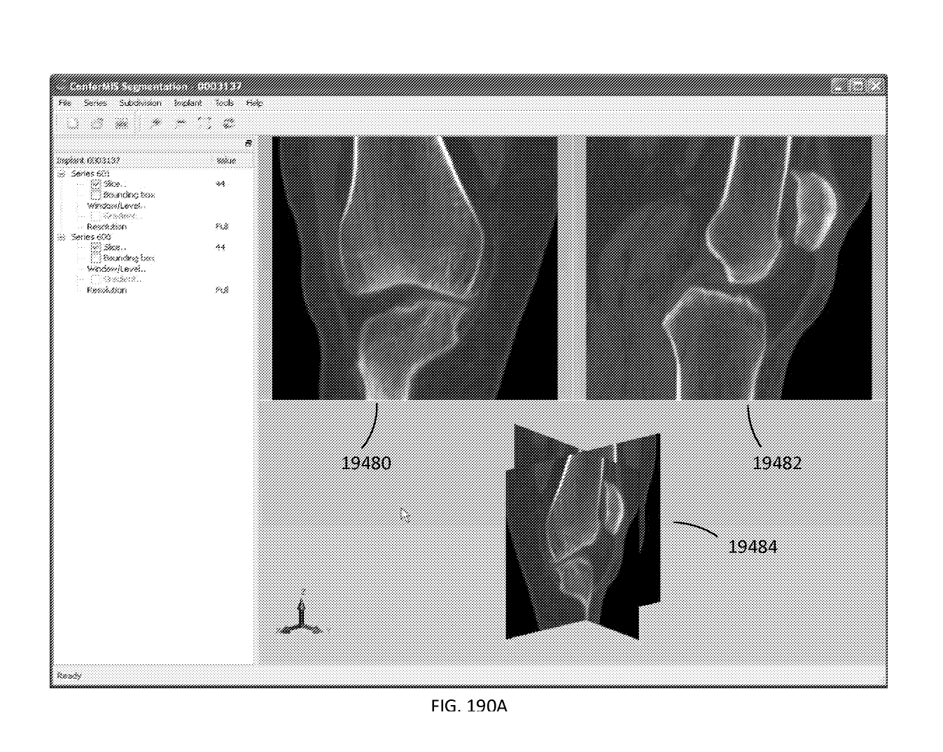

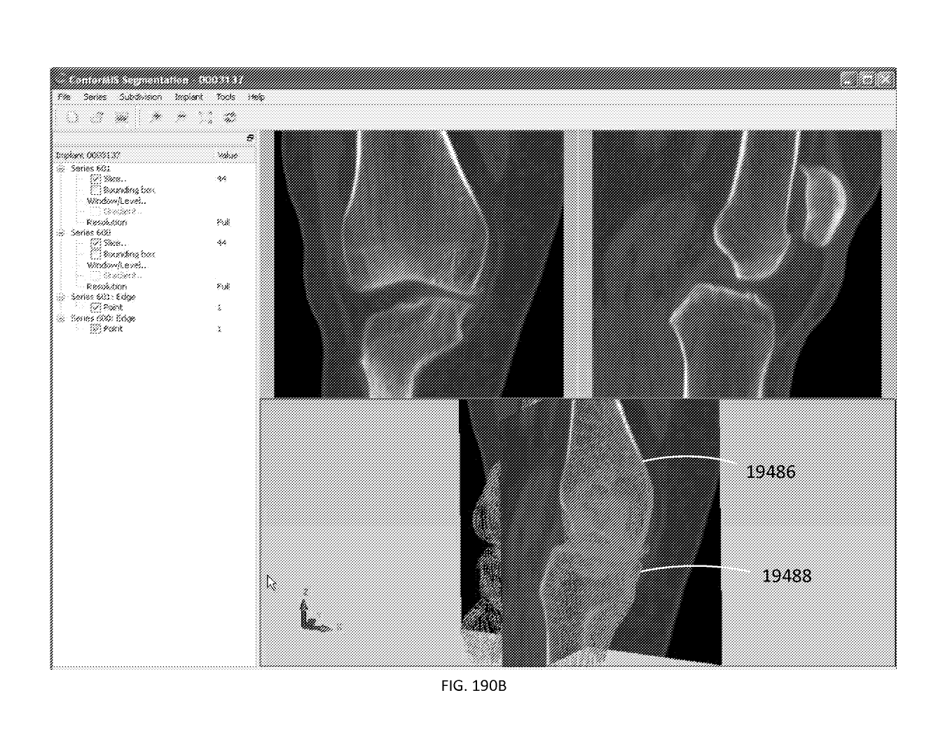

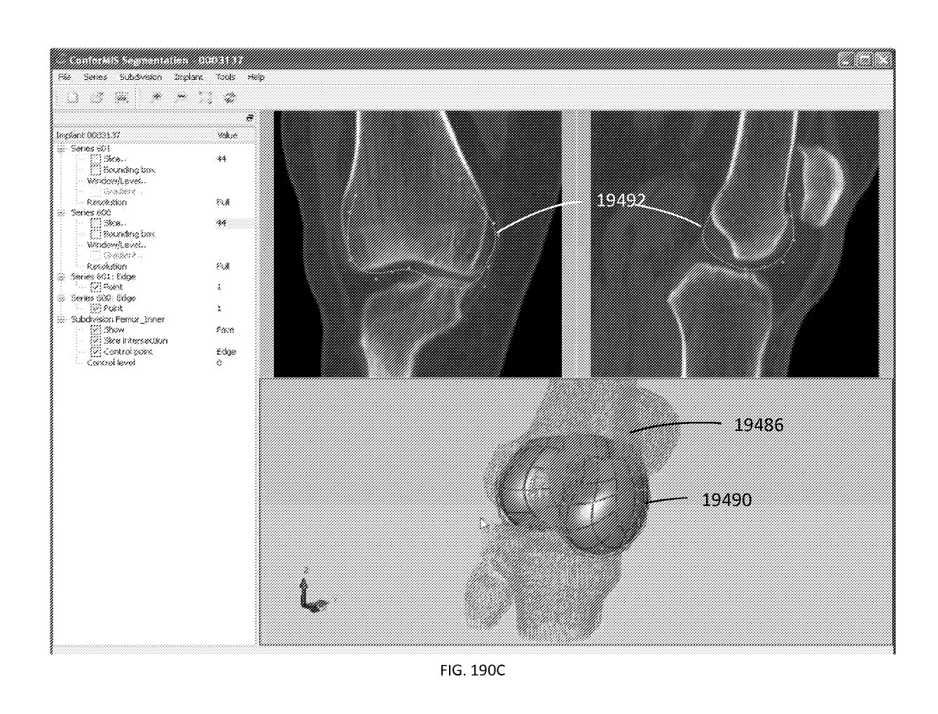

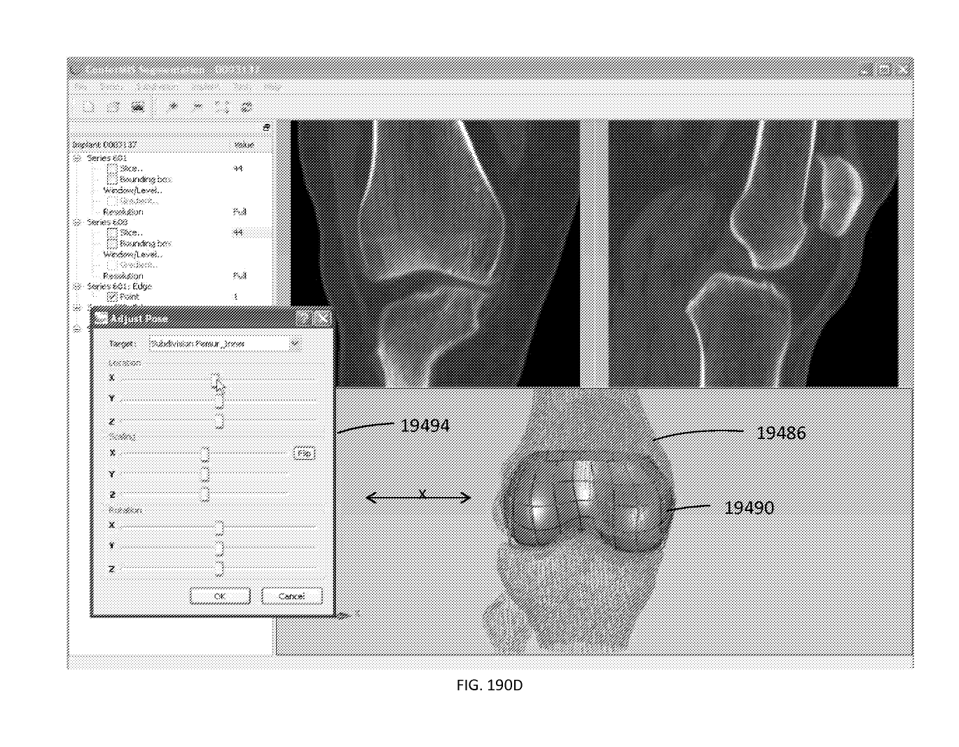

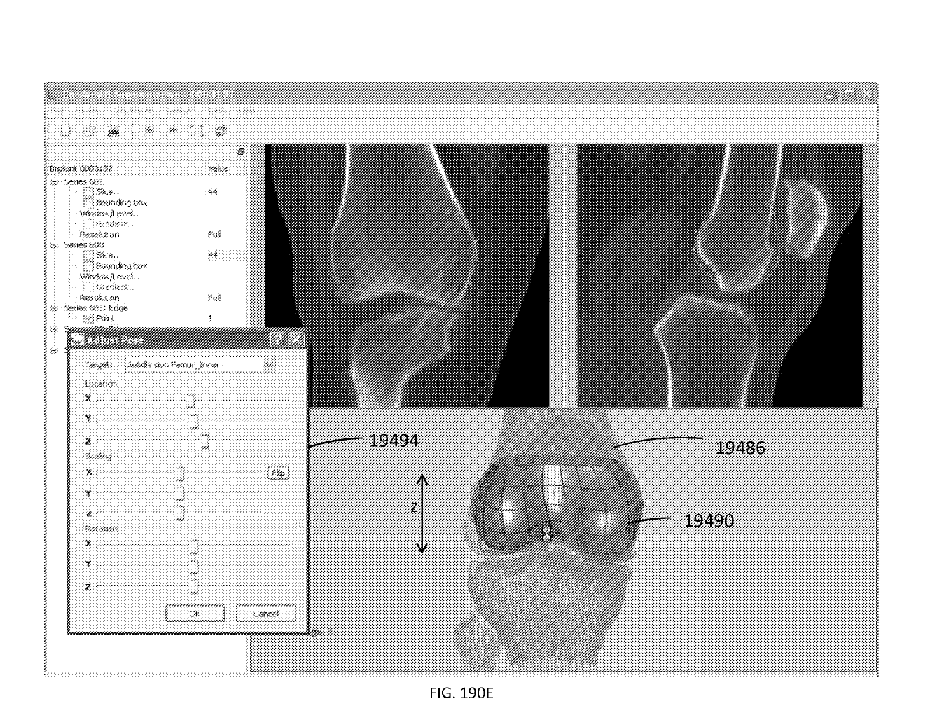

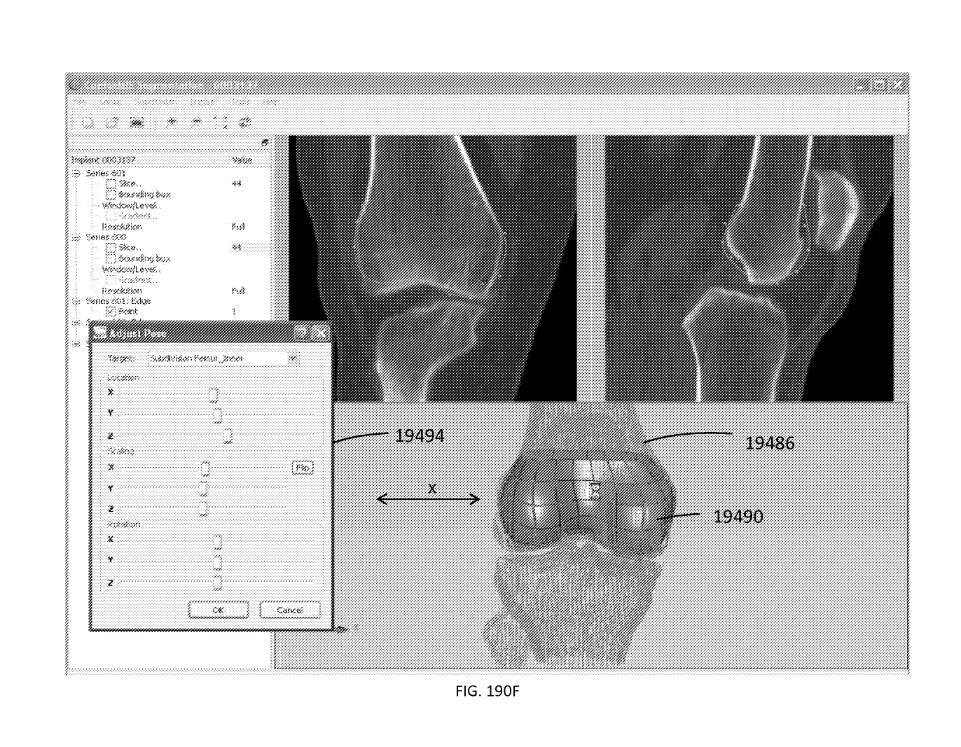

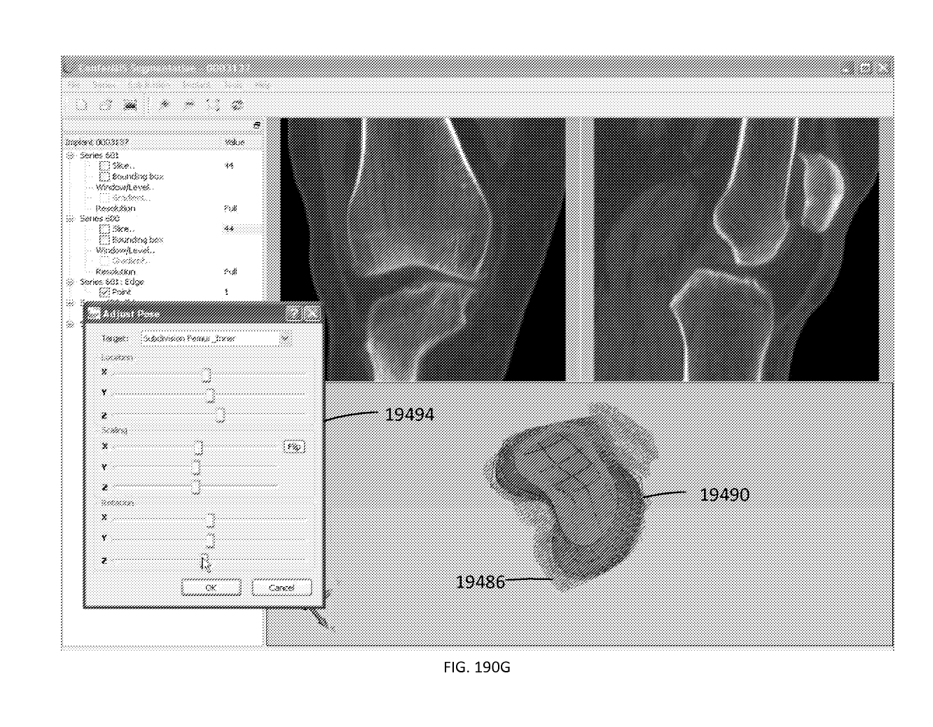

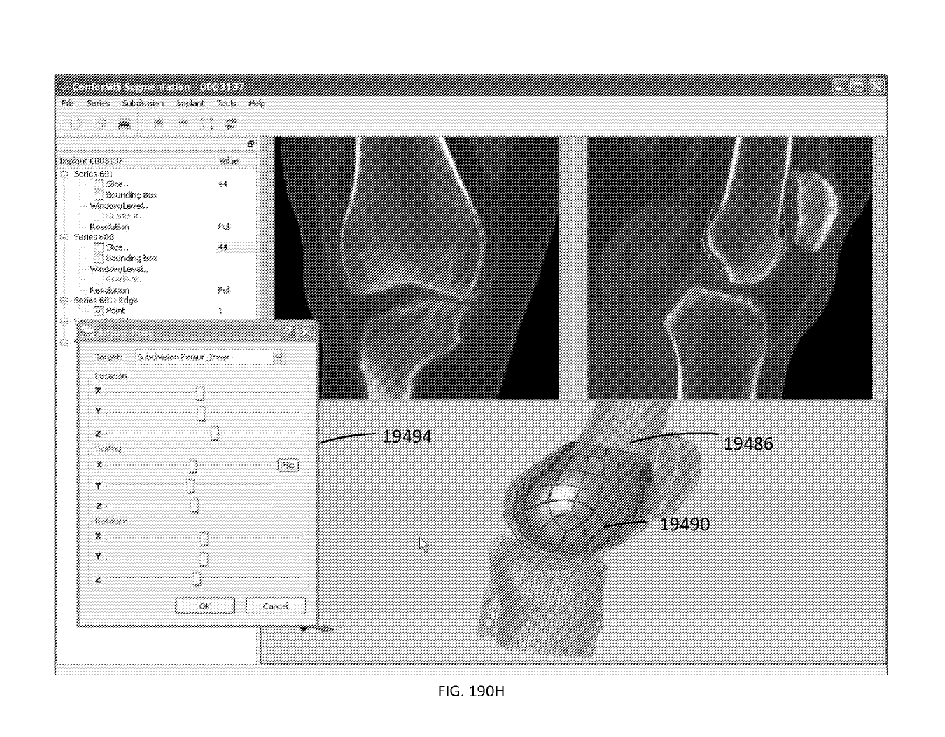

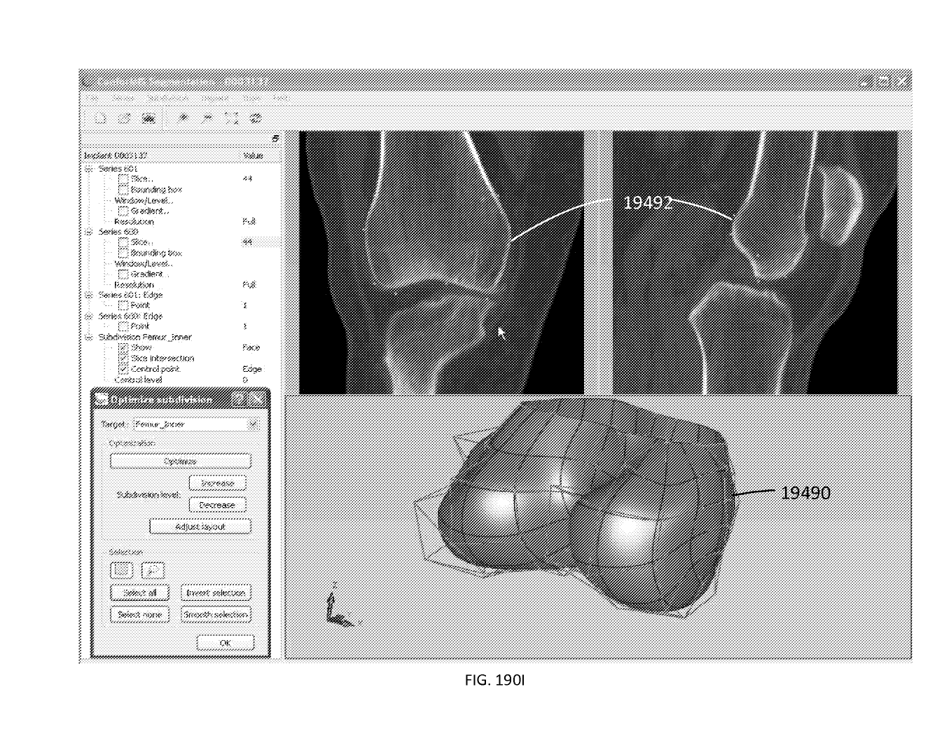

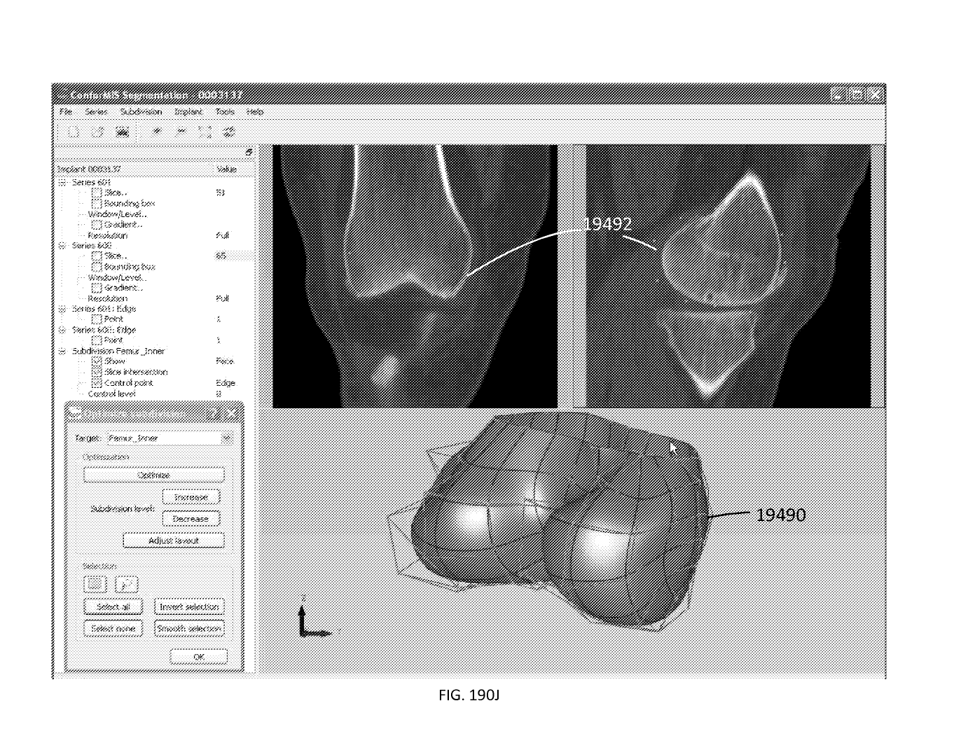

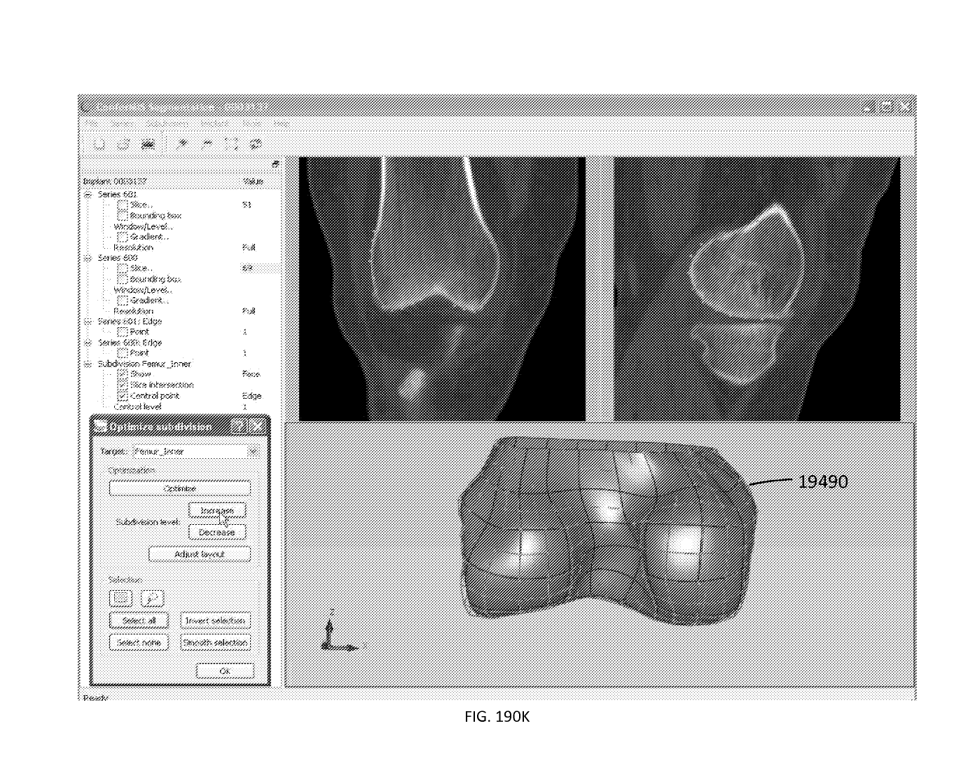

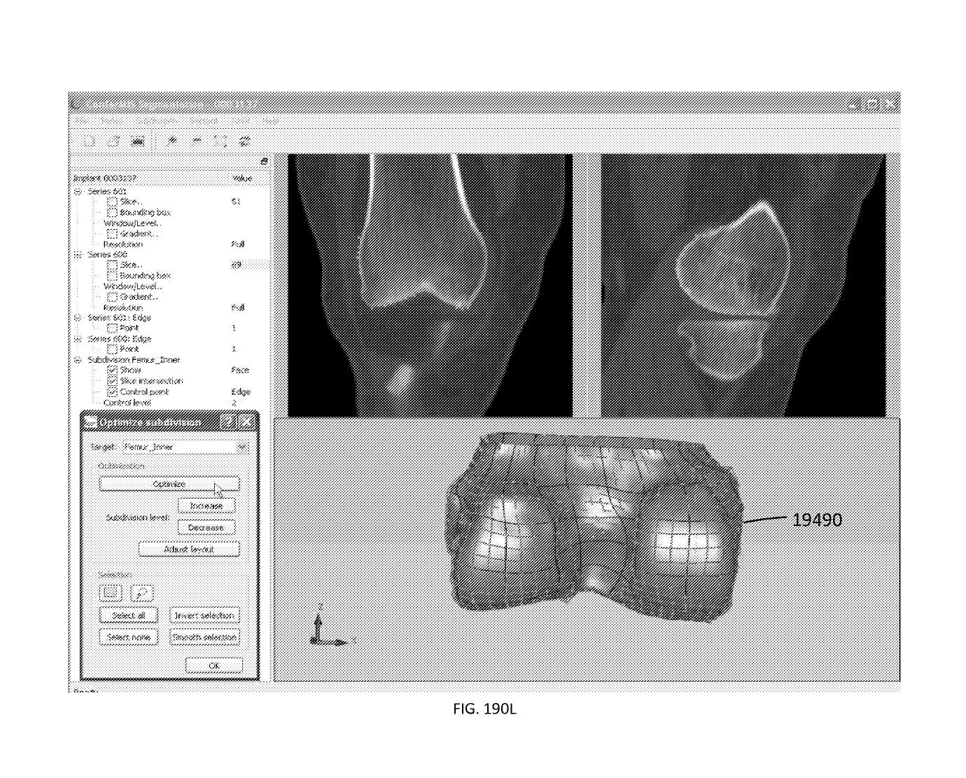

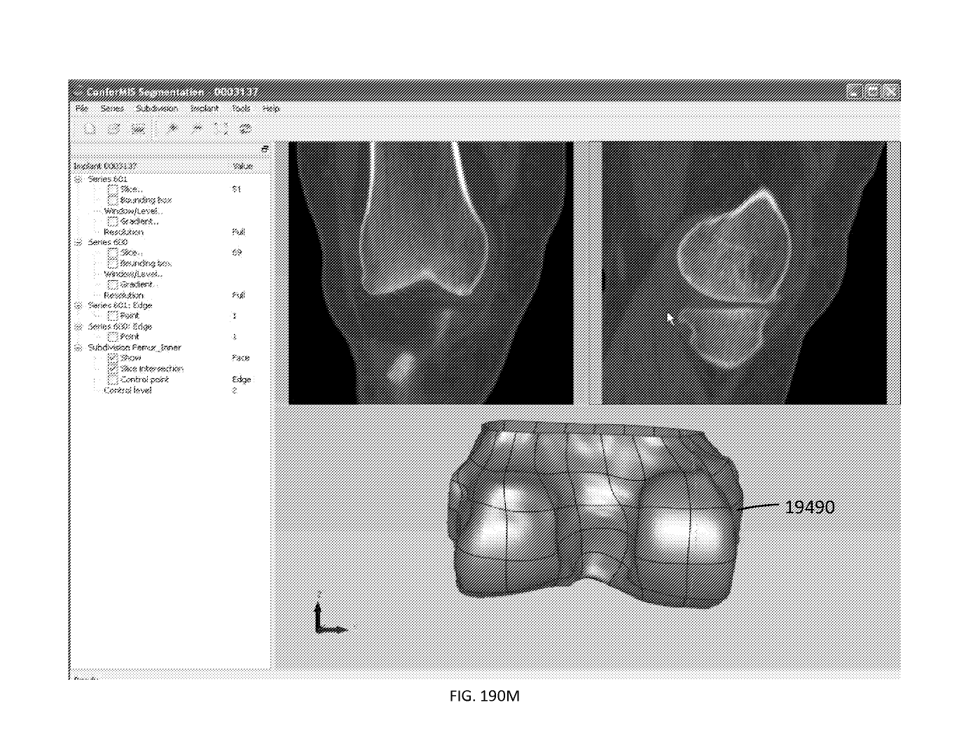

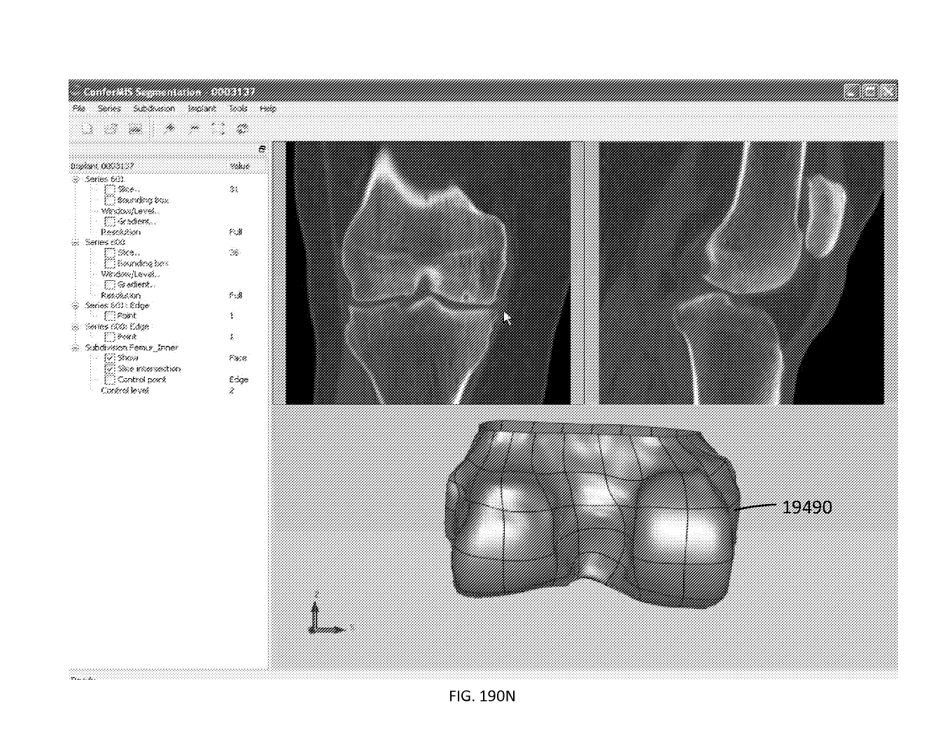

FIG. 189 shows a flowchart of steps in certain embodiments of a deformable segmentation method. The steps include one or more of collecting multiple images of a patient's biological structure 19460; optionally approximating a biological feature of interest 19464; applying a template model to the approximate biological feature of interest 19468; optionally roughly fitting the template model to the approximate biological feature 19472; and then precisely fitting the template model to the collection of multiple images 19476. Similar to the method described above, one or more of these steps 19460, 19464, 19468, 19472, 19476 can be repeated 19461, 19465, 19469, 19473, 19477 as often as desired to achieve the desired result. Moreover, the steps can be repeated reiteratively 19462, 19466, 19470, 19474, 19478. FIGS. 190A-190O show exemplary images from a computer program that applies an embodiment of the deformable segmentation method.

In one step 19460, multiple images can be collected for processing together, for example, the images can be processed together in a single event rather than individually. As illustrated in FIG. 190A, a computer program can be used to load, view and/or process the multiple images as one or more views into one or more 3D image data stacks, for example coronal, sagittal or axial views. In the figure, a series of coronal image slices 19480 and a series of sagittal image slices 19482 can be viewed as separate stacks or decks of 2D images. These stacks of images can result from separate image scans or can be different views of the same scan. In addition, any two or more images can be combined 19484 to provide a 3D image.

In another step 19464, a biological feature of interest is approximated from the multiple images. FIG. 190B illustrates the approximated biological features of a femoral surface 19486 and a tibial surface 19488. The approximated surface can be provided by the method described above, for example, by detecting edges in each image based on relative grayscale or intensity changes, and then combining the image data. This step is optional.

In another step 19468, a template model can be applied to the approximate biological feature or directly to the combined image data stack. FIG. 190C illustrates a femoral template model 19490 applied to the approximate femoral surface 19486. In applying a template model, the software can select one or more initial best fit template models. Template models can be available, for example, from a library of models, for example, collected from one or more previous assessments.

As shown by the template outline 19492 in the 2D images, the femoral template 19490 initially is not a substantial match for the approximate femoral surface 19486. This match can be improved by making global and local adjustments. Global adjustments align the template by performing operations such as rotating, translating or scaling. Local adjustments deform the surface representation of the template in certain subregions. In an optional step 19472, the software can roughly fit the template model to the biological feature of interest or directly to the image data stack. FIG. 190D-190G illustrate the femoral template model 19490 being roughly adjusted to best-fit the approximate femoral surface 19486. As shown in the figures, if non-automated intervention is required or desired, a user can perform the adjustments using a control panel 19494, although in various embodiments such adjustments can be performed by the automated system. Adjustments can include, for example, adjusting the location of the template in one or more dimensions; adjusting the scale (e.g., size) of the template in one or more dimensions; and adjusting the rotation of the template in one or more dimensions. As shown in the control panel 19494 as position changes to the user-controlled knobs relative to their initial center positions, FIG. 190D illustrates a user adjustment to the location of the template model in the x axis (e.g., in the M-L direction); FIG. 190E illustrates a user adjustment to the location of the template model in the z axis (e.g., in the proximal-distal direction); FIG. 190F illustrates a user adjustment to the scale (i.e., size) of the template model in the x axis; and FIG. 190G illustrates a user adjustment to rotation of the template model about the z-axis (the axis perpendicular to the view). These adjustments can be performed in any order and repeated as desired to achieve the best rough fit of the template with the approximate biological feature. In other embodiments, the software can automatically determine the initial best fit of the template model to the biological feature of interest or the image data. This can be achieved by finding the scaling, rotation and translation parameters that result in the closest fit of the template to the structure of interest, for example using a multidimensional optimization algorithm. FIG. 190H illustrates the rough fit of the template to the approximate surface following these adjustments.

In another step 19476, the model template can be precisely fit to the collection of multiple images (rather than, in the method described above, independently processing each image). As shown in FIG. 190I, the surface quadrangles or “patches” of surface data representation of the femoral template 19490 can be deformed to match the surface(s) across the entire collection of images. In certain embodiments, the template patches can be deformed to directly fit the radiographic or tomographic image data (e.g., voxel data) rather than any subsequently processed data, for example, data points representing multiple voxels or data compatible with a computer monitor. Currently, radiographic or tomographic images can include much higher gray value resolution (e.g., can assign one of a much greater number of unique shades of gray to each pixel or voxel) than data compatible with a computer monitor. Accordingly, by deforming the template to directly fit the radiographic images, a high degree of resolution can be maintained, which can provide a highly precise model.

The points or dots shown in association with the template outline 19492 represent control points that can be used by automated system or a technician to alter the outline and surface of the template. By moving a control point, the user can manually alter and deform adjacent sections of the surface data representation of the template, and the resulting alterations and deformations can appear in both the 2D outline view and in the 3D view of the template. In another embodiment, the software can optimize the position of the control points and thus the fit of the surface automatically using various criteria, for example gray values or gray value gradients in the image data or smoothness and continuity constraints in the surface data representation.

FIG. 190I illustrates the femur template model 19490 being deformed to fit the edges of the femur in the collection of radiographic images. As shown in the 2D images, the model surface 19492 fits precisely with the outline of the femur in the images shown. As indicated by FIG. 190J, the model surface 19492 can be viewed and/or visually checked across any of the 2D image slices. The precision of the model can be enhanced further by adding additional surface detail (e.g., additional parametric surface quadrangles or patches) to the model and repeating the deforming step. This process can be reiterated, as indicated by the increasing number of polygon surfaces in FIGS. 190K and 190L, to provide a highly precise patient-specific model. As shown in FIGS. 190M-190O, control points can be hidden and the patient-specific model can be viewed in 2D comparison to any of the images, or in 3D 19490.

### Modeling of Anatomical Data

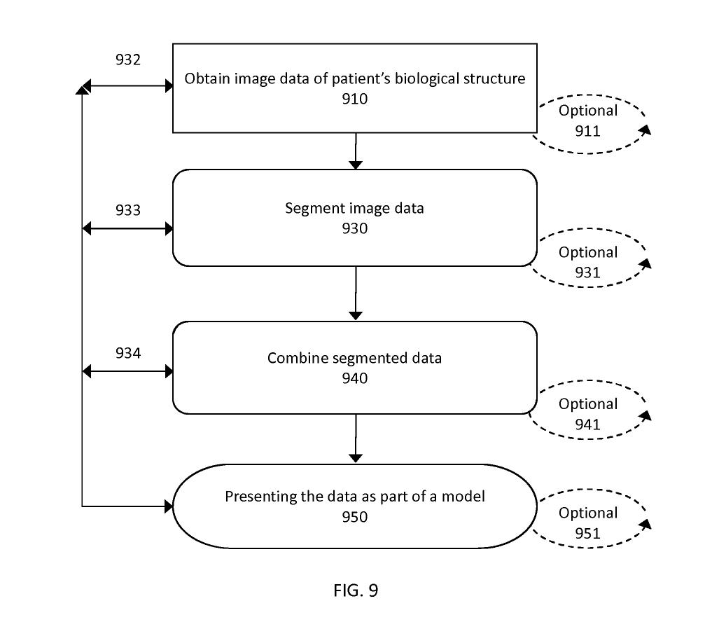

In certain embodiments, one or more models of at least a portion of a patient's joint can be generated. Specifically, the patient-specific data and/or measurements described herein can be used to generate a model that includes at least a portion of the patient's joint. Various methods can be used to generate a model. As illustrated in FIG. 9, in certain embodiments the method of generating a model of a patient's joint (and/or a resection cut, drill hole, guide tool, and/or implant component) or other biological feature (and/or a patient-specific feature of a guide tool or implant component) can include one or more of the steps of obtaining image data of a patient's biological structure 910; segmenting the image data 930; combining the segmented data 940; and presenting the data as part of a model 950.

Image data can be obtained 910 from near or within the patient's biological structure of interest. For example, pixel or voxel data from one or more radiographic or tomographic images of a patient's joint can be obtained, for example, using computed or magnetic resonance tomography. Other imaging modalities known in the art such as ultrasound, laser imaging, PET, SPECT, radiography including digital radiography, digital tomosynthesis, cone beam CT, and contrast enhanced imaging can be used. In this or a subsequent step, one or more of the pixels or voxels can be converted into one or a set of values. For example, a single pixel/voxel or a group of pixel/voxels can be converted to coordinate values, e.g., a point in a 2D or 3D coordinate system. The set of values also can include a value corresponding to the pixel/voxel intensity or relative grayscale color. Moreover, the set of values can include information about neighboring pixels or voxels, for example, information corresponding to relative intensity or grayscale color and or information corresponding to relative position.

Then, the image data can be segmented 930 to identify those data corresponding to a particular biological feature of interest. For example, as shown in FIG. 188A, image data can be used to identify the edges of a biological structure, in this case, the surface outline for each of the patient's femur and tibia. As shown, the distinctive transition in color intensity or grayscale 19000 at the surface of the structure can be used to identify pixels, voxels, corresponding data points, a continuous line, and/or surface data representing the surface of the biological structure. This step can be performed automatically (for example, by a computer program operator function) or manually (for example, by a clinician or technician), or by various combinations of the two.

Optionally, the segmented data can be combined 940. For example, in a single image segmented and selected reference points (e.g., derived from pixels or voxels) and/or other data can be combined to create a line representing the surface outline of a biological structure. Moreover, as shown in FIGS. 188A and 188B, the segmented and selected data from multiple images can be combined to create a 3D representation of the biological structure. Alternatively, the images can be combined to form a 3D data set, from which the 3D representation of the biological structure can be derived directly using a 3D segmentation technique, for example an active surface or active shape model algorithm or other model based or surface fitting algorithm.

Optionally, the 3D representation of the biological structure can be generated or manipulated, for example, smoothed or corrected, for example, by employing a 3D polygon surface, a subdivision surface or parametric surface, for example, a non-uniform rational B-spline (NURBS) surface. For a description of various parametric surface representations see, for example Foley, J. D. et al., Computer Graphics: Principles and Practice in C; Addison-Wesley, 2nd edition (1995). Various methods are available for creating a parametric surface. For example, the 3D representation can be converted directly into a parametric surface, for example, by connecting data points to create a surface of polygons and applying rules for polygon curvatures, surface curvatures, and other features. Alternatively, a parametric surface can be best-fit to the 3D representation, for example, using publicly available software such as Geomagic® software (Research Triangle Park, N.C.).

Then, the data can be presented as part of a model 950, for example, a patient-specific virtual model that includes the biological feature of interest. Optionally, the data associated with one or more biological features can be transferred to one or more resection cuts, drill holes, guide tools, and/or implant components, which also can be included as part of the same model or in a different model. As will be described below, the virtual model(s) can be used to generate one or more patient-adapted guide tools and/or implant components for surgical use, for example, using computer-aided design (CAD) software and/or one or more of the several manufacturing techniques described below, optionally in conjunction with computer-aided manufacturing (CAM) software.

As will be appreciated by those of skill in the art, one or more of these steps 910, 930, 940, 950 can be repeated 911, 931, 941, 951 as often as desired to achieve the desired result. Moreover, the steps can be repeated reiteratively 932, 933, 934. Moreover, the program could proceed directly 933 from the step of segmenting image data 930 to presenting and/or utilizing the data as part of a model 950. Data, models and/or any related guide tools or implant components can be collected in one or more libraries for subsequent use for the same patient or for a different patient (e.g., a different patient with similar data).

In various embodiments, the various anatomical features of a given bone or anatomical structure, such as the tibia (i.e., anterior-posterior and/or medial-lateral dimensions, perimeters, medial/lateral slope, shape, tibial spine height, and other features) can be measured, modeled, and then compared to and/or modified based on a database of one or more “normal” or “healthy” tibial measurement and/or models, with the resulting information used to choose or design a desired implant shape, size and placement. In a similar manner, the various anatomical features of any joint can be measured and then compared/modified based on a database of “healthy” or otherwise appropriate measurements of appropriate joints, including those of the medial condyle, a lateral condyle, a trochlea, a medial tibia, a lateral tibia, the entire tibia, a medial patella, a lateral patella, an entire patella, a medial trochlea, a central trochlea, a lateral trochlea, a portion of a femoral head, an entire femoral head, a portion of an acetabulum, an entire acetabulum, a portion of a glenoid, an entire glenoid, a portion of a humeral head, an entire humeral head, a portion of an ankle joint, an entire ankle joint, and/or a portion or an entire elbow, wrist, hand, finger, spine, or facet joint.

It may also be desirable to model various of the patient measurements (especially non-load-bearing measurements as described above) to simulate the targeted joint and surrounding anatomy virtually. Such simulations can include virtually modeling the alignment and load bearing condition of the joint and surrounding anatomical structures for the patient standing and/or moving (i.e., walking, running, jumping, squatting, kneeling, walking up and down stairs or inclines/declines, picking up objects, etc.). Such simulations can be used to obtain valuable anatomical, biomechanical and kinematic data including the loaded condition of various joint components, component positions, component movement, joint and/or surrounding tissue anatomical or biomechanical constraints or limitations, as well as estimated mechanical axes in one or more directions (i.e., coronal, sagittal or combinations thereof). This information could then be utilized (alone or in combination with other data described herein) to design various features of a joint resurfacing/replacement implant. This method can be incorporated in the various embodiments described herein as additional patient measurement and anatomical/joint modeling and design data. This analysis is applicable to many different joints, including those of the medial condyle, a lateral condyle, a trochlea, a medial tibia, a lateral tibia, the entire tibia, a medial patella, a lateral patella, an entire patella, a medial trochlea, a central trochlea, a lateral trochlea, a portion of a femoral head, an entire femoral head, a portion of an acetabulum, an entire acetabulum, a portion of a glenoid, an entire glenoid, a portion of a humeral head, an entire humeral head, a portion of an ankle joint, an entire ankle joint, and/or a portion or an entire elbow, wrist, hand, finger, spine, or facet joint.

Modeling Joint Structures and/or Correcting Defects

In certain embodiments, the reference points and/or measurements described above can be processed using mathematical functions or other data sources (i.e., models, databases, etc) to derive virtual, corrected features, which may represent a restored, ideal or desired feature from which a patient-adapted implant component can be designed. For example, one or more features, such as surfaces or dimensions of a biological structure can be modeled, altered, added to, changed, deformed, eliminated, corrected and/or otherwise manipulated (collectively referred to herein as “variation” of an existing surface or structure within the joint). While it is described in the knee, these embodiments can be applied to any joint or joint surface in the body, e.g., a knee, hip, ankle, foot, toe, shoulder, elbow, wrist, hand, and a spine or spinal joints.

Variation of the joint or portions of the joint can include, without limitation, variation of one or more external surfaces, internal surfaces, joint-facing surfaces, uncut surfaces, cut surfaces, altered surfaces, and/or partial surfaces as well as osteophytes, subchondral cysts, geodes or areas of eburnation, joint flattening, contour irregularity, and loss of normal shape. The surface or structure can be or reflect any surface or structure in the joint, including, without limitation, bone surfaces, ridges, plateaus, cartilage surfaces, ligament surfaces, or other surfaces or structures. The surface or structure derived can be an approximation of a healthy joint surface or structure or can be another variation. The surface or structure can be made to include pathological alterations of the joint. The surface or structure also can be made whereby the pathological joint changes are virtually removed in whole or in part.

Once one or more reference points, measurements, structures, surfaces, models, or combinations thereof have been selected or derived, the resultant shape can be varied, deformed or corrected, if desired. In certain embodiments, the variation can be used to select and/or design an implant component having an ideal or optimized feature or shape, e.g., corresponding to the deformed or corrected joint feature or shape. For example, in one application of this embodiment, the ideal or optimized implant shape reflects the shape of the patient's joint before he or she developed arthritis.

Alternatively or in addition, the variation can be used to select and/or design a patient-adapted surgical procedure to address the deformity or abnormality. For example, the variation can include surgical alterations to the joint, such as virtual resection cuts, virtual drill holes, virtual removal of osteophytes, and/or virtual building of structural support in the joint deemed necessary or beneficial to a desired final outcome for a patient.

Corrections can be used to address osteophytes, subchondral voids, and other patient-specific defects or abnormalities. In the case of osteophytes, a design for the bone or joint facing surface of an implant component or guide tool can be selected and/or designed after the osteophyte has been virtually removed. Alternatively, the osteophyte can be integrated into the shape of the bone or joint facing surface of the implant component or guide tool.

For example, a tibial component can be designed either before or after virtual removal of various features of the tibial bone have been accomplished. In one embodiment, the initial design and placement of the tibial tray and associated components can be planned and accomplished utilizing information directly taken from the patient's natural anatomy. In various other embodiments, the design and placement of the tibial components can be planned and accomplished after virtual removal of various bone portions, including the removal of one or more cut planes (to accommodate the tibial implant) as well as the virtual removal of various potentially-interfering structures (i.e., overhanging osteophytes, etc.) and/or the virtual filling of voids, etc. Prior virtual removal/filling of such structures can facilitate and improve the design, planning and placement of tibial components, and prevent anatomic distortion from significantly affecting the final design and placement of the tibial components. For example, once one or more tibial cut planes has been virtually removed, the size, shape and rotation angle of a tibial implant component can be more accurately determined from the virtually surface, as compared to determining the size, shape and/or tibial rotation angle of an implant from the natural tibial anatomy prior to such cuts. In a similar manner, structures such as overhanging osteophytes can be virtually removed (either alone or in addition to virtual removal of the tibial cut plane(s)), with the tibial implant structure and placement (i.e., tibial implant size, shape and/or tibial rotation, etc.) subsequently planned. Of course, virtually any undesirable anatomical features or deformity, including (but not limited to) altered bone axes, flattening, potholes, cysts, scar tissue, osteophytes, tumors and/or bone spurs may be similarly virtually removed and then implant design and placement can be planned.

Similarly, to address a subchondral void, a selection and/or design for the bone-facing surface of an implant component can be derived after the void has been virtually removed (e.g., filled). Alternatively, the subchondral void can be integrated into the shape of the bone-facing surface of the implant component.

In addition to osteophytes and subchondral voids, the methods, surgical strategies, guide tools, and implant components described herein can be used to address various other patient-specific joint defects or phenomena. In certain embodiments, correction can include the virtual removal of tissue, for example, to address an articular defect, to remove subchondral cysts, and/or to remove diseased or damaged tissue (e.g., cartilage, bone, or other types of tissue), such as osteochondritic tissue, necrotic tissue, and/or torn tissue. In such embodiments, the correction can include the virtual removal or other modification of the tissue (e.g., the tissue corresponding to the defect, cyst, disease, or damage) and the bone-facing surface of the implant component can be derived after the tissue has been virtually removed/modified. In certain embodiments, the implant component can be selected and/or designed to include a thickness or other features that substantially matches the removed/modified tissue and/or optimizes one or more parameters of the joint. Optionally, a surgical strategy and/or one or more guide tools can be selected and/or designed to reflect the correction and correspond to the implant component.

Various methods of more accurately modeling a target anatomical site can be utilized prior to designing and placing an implant component. For example, in the case of designing and placing a tibial implant, it may be desirous to incorporate additional virtual criteria into the virtual anatomic model of the targeted anatomy prior to designing and placing the tibial implant component. (One or more of the following, in any combination, may be incorporated with varying results.)

Tibial plateau (leave uncut or virtually cut along one or more planes in model)

Osteophytes (leave intact or virtually remove in model)

Voids (leave intact or virtually fill in model)

Tibial tubercle (incorporate in virtual model or ignore this anatomy)

Femoral anatomic landmarks (incorporate in virtual model or ignore)

Anatomic or biomechanical axes (incorporate in virtual model or ignore)

Femoral component orientation (incorporate in virtual model or ignore)

After creation of the virtual anatomic model, incorporating one or more of the previous virtual variations in various combinations, the design and placement of the tibial implant (i.e., size, shape, thickness and/or tibial tray rotation angle and orientation) can be more accurately determined. Similarly, the design and placement of a femoral implant (i.e., size, shape, thickness and/or femoral component rotation angle and orientation) can be more accurately determined. Likewise, the design and placement of a other implant components (i.e., size, shape, thickness and/or component rotation angle and orientation), e.g., for acetabular or femoral head resurfacing or replacement, glenoid or humeral head resurfacing or replacement, elbow resurfacing or replacement, wrist resurfacing or replacement, hand resurfacing or replacement, ankle resurfacing or replacement, for resurfacing or replacement can be more accurately determined.

In certain embodiments, a correction can include the virtual addition of tissue or material, for example, to address an articular defect, loss of ligament stability, and/or a bone stock deficiency, such as a flattened articular surface that should be round. In certain embodiments, the additional material may be virtually added (and optionally then added in surgery) using filler materials such as bone cement, bone graft material, and/or other bone fillers. Alternatively or in addition, the additional material may be virtually added as part of the implant component, for example, by using a bone-facing surface and/or component thickness that match the correction or by otherwise integrating the correction into the shape of the implant component. Then, the joint-facing and/or other features of the implant can be derived. This correction can be designed to re-establish a near normal shape for the patient. Alternatively, the correction can be designed to establish a standardized shape or surface for the patient.

In certain embodiments, the patient's abnormal or flattened articular surface can be integrated into the shape of the implant component, for example, the bone-facing surface of the implant component can be designed to substantially negatively-match the abnormal or flattened surface, at least in part, and the thickness of the implant can be designed to establish the patient's healthy or an optimum position of the patient's structure in the joint. Moreover, the joint-facing surface of the implant component also can be designed to re-establish a near normal anatomic shape reflecting, for example, at least in part the shape of normal cartilage or subchondral bone. Alternatively, it can be designed to establish a standardized shape.

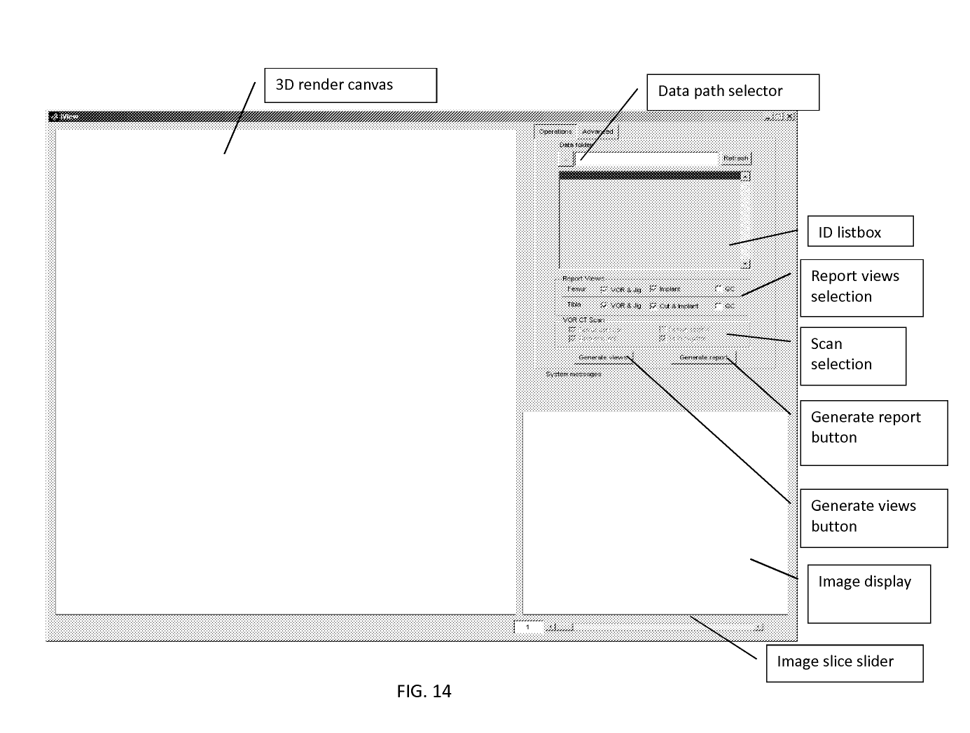

Computer software programs to generate models of patient-specific renderings of implant assembly and defects (e.g., osteophyte structures), together with bone models, to aid in surgery planning can be developed using various publicly available programming environments and languages, for example, Matlab 7.3 and Matlab Compiler 4.5, C++ or Java. In certain embodiments, the computer software program can have a user interface that includes one or more of the components identified in FIG. 14. Alternatively, one or more off-the-shelf applications can be used to generate the models, such as SolidWorks, Rhinoceros, 3D Slicer or Amira.

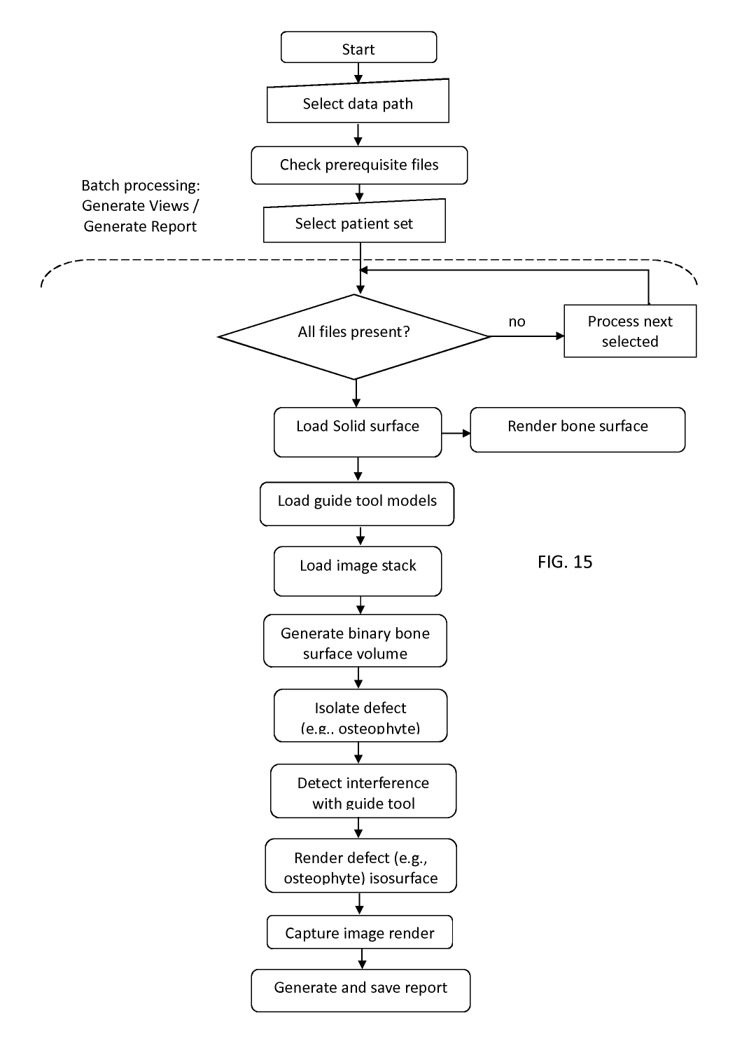

An illustrative flow chart of the high level processes of an exemplary computer software program is shown in FIG. 15. Briefly, a data path associated with one or more patient folders that include data files, for example, patient-specific CT images, solid models, and segmentation images, is selected. The data files associated with the data path can be checked, for example, using file filters, to confirm that all data files are present. For example, in generating models for a knee implant, a data path can confirm the presence of one, several, or all coronal CT data files, sagittal CT data files, a femoral solid model data file, a tibial solid model data file, a femoral guide tool model, a tibial guide tool model, a femoral coronal segmentation model, a femoral sagittal segmentation model, a tibial coronal segmentation model, and a tibial sagittal segmentation model. If the filter check identifies a missing file, the user can be notified. In certain instances, for example, if a tibial or femoral guide tool model file is unavailable, the user may elect to continue the process without certain steps, for example, without guide tool—defect (e.g., osteophyte) interference analysis.

Next, a patient-specific bone-surface model is obtained and/or rendered. The bone surface model provides basic patient-specific features of the patient's biological structure and serves as a reference for comparison against a model or value that includes the defect(s) of interest. As an illustrative example, previously generated patient-specific files, for example, STL files exported from “SOLID” IGES files in SolidWorks, can be loaded, for example, as triangulation points with sequence indices and normal vectors. The triangles then can be rendered (e.g., using Matlab TRISURF function) to supply or generate the bone-surface model. The bone surface model can include corrections of defects, such as osteophytes removed from the bone. In a similar fashion, one or more guide tool models can be obtained and/or rendered.

Next, a patient-specific model or values of the patient's biological feature that include the defect of interest can be obtained and/or rendered. For example, patient-specific defects, such as osteophytes, can be identified from analysis of the patient's segmentation images and corresponding CT scan images. The transformation matrix of scanner coordinate space to image matrix space can be calculated from image slice positions (e.g., the first and last image slice positions). Then, patient-specific segmentation images for the corresponding scan direction can be assessed, along with CT image slices that correspond to the loaded segmentation images. Images can be processed slice by slice and, using selected threshold values (e.g., intensity thresholds, Hounsfield unit thresholds, or neighboring pixel/voxel value thresholds), pixels and/or voxels corresponding to the defects of interest (e.g., osteophytes) can be identified. The identified voxels can provide a binary bone surface volume that includes the defects of interest as part of the surface of the patient's biological structure. Various masks can be employed to mask out features that are not of interest, for example, an adjacent biological surface. In some instances, masking can generate apparent unattached portions of an osteophyte defect, for example, when a mask covers a portion of an osteophyte extension.

Next, the defects of interest can be isolated by comparing the model that does not include the defects of interest (e.g., bone-surface model) with the model or value that does include the defects of interest (e.g., the binary bone surface volume). For example, the triangulation points of the bone surface model can be transformed onto an image volume space to obtain a binary representation of the model. This volume binary can be dilated and thinned to obtain a binary bone model. The binary bone model then can serve as a mask to the binary bone surface volume to identify defect volume separate from the binary bone surface volume. For example, for osteophyte detection, the osteophyte volume (e.g., osteophyte binary volume), as well as the osteophyte position and attachment surface area, can be distinguished from the patient's biological structure using this comparative analysis. Various thresholds and filters can be applied to remove noise and/or enhance defect detection in this step. For example, structures that have less than a minimum voxel volume (e.g., less than 100 voxels) can be removed. Alternatively, or in addition, rules can be added to “reattach” any portion of an osteophyte defect that appears unattached, e.g., due to a masking step.

In an alternative approach, surface data can be used instead of voxel or volume data when comparing the bone surface model with corrected defects and the patient's actual bone surface. The bone surface model, for example, can be loaded as a mesh surface (e.g., in an STL file) or a parametric surface (e.g., in an IGES file) without conversion to volumetric voxel data. The patient's natural bone surface can be derived from the medical image data (e.g., CT data) using, for example, a marching cubes or isosurface algorithm, resulting in a second surface data set. The bone surface model and the natural bone surface can be compared, for example, by calculating intersection between the two surfaces.

Next, optionally, the models can be used to detect interference between any defect volume and the placement of one or more guide tools and/or implant components. For example, guide tool model triangulation points can be transformed onto an image volume space to obtain a binary representation of the guide tool. The binary structure then can be manipulated (e.g., dilated and eroded using voxel balls having pre-set diameters) to obtain a solid field mask. The solid field mask can be compared against the defect volume, for example, the osteophyte binary volume, to identify interfering defect volume, for example, interfering osteophyte binary volume. In this way, interfering defect volume and non-interfering defect volume can be determined (e.g., using Matlab ISOSURFACE function), for example, using representative colors, shading or some other distinguishing features in a model. The resulting model image can be rendered on a virtual rendering canvas (e.g., using Matlab GETFRAME function) and saved onto a computer-readable medium.

Finally, optionally, one or more combinations of model features can be combined into one or models or sets of models that convey desired information to the surgeon or clinician. For example, patient-specific bone models can be combined with any number of defects or defect types, any number of resection cuts, any number of drill holes, any number of axes, any number of guide tools, and/or any number of implant components to convey as much information as desired to the surgeon or clinician. The patient-specific bone model can model any biological structure, for example, any one or more (or portion of) a femoral head and/or an acetabulum; a distal femur, one or both femoral condyle(s), and/or a tibial plateau; a trochlea and/or a patella; a glenoid and/or a humeral head; a talar dome and/or a tibial plafond; a distal humerus, a radial head, and/or an ulna; and a radius and/or a scaphoid. Defects that can be combined with a patient-specific bone model can include, for example, osteophytes, voids, subchondral cysts, articular shape defects (e.g., rounded or flattened articular surfaces or surface portions), varus or valgus deformities, or any other deformities known to those in the art.

The models can include virtual corrections reflecting a surgical plan, such as one or more of removed osteophytes, cut planes, drill holes, realignments of mechanical or anatomical axes. The models can include comparison views demonstrating the anatomical situation before and after applying the planned correction. The individual steps of the surgical plan can also be illustrated in a series of step-by-step images wherein each image shows a different step of the surgical procedure.

The models can be presented to the surgeon as a printed or digital set of images. In another embodiment, the models are transmitted to the surgeon as a digital file, which the surgeon can display on a local computer. The digital file can contain image renderings of the models. Alternatively, the models can be displayed in an animation or video. The models can also be presented as a 3D model that is interactively rendered on the surgeon's computer. The models can, for example, be rotated to be viewed from different angles. Different components of the models, such as bone surfaces, defects, resection cuts, axes, guide tools or implants, can be turned on and off collectively or individually to illustrate or simulate the individual patient's surgical plan. The 3D model can be transmitted to the surgeon in a variety of formats, for example in Adobe 3D PDF or as a SolidWorks eDrawing.

### Modeling Proper Limb Alignment

Proper joint and limb function depend on correct limb alignment. For example, in repairing a knee joint with one or more knee implant components, optimal functioning of the new knee may depend on the correct alignment of the anatomical and/or mechanical axes of the lower extremity. Accordingly, an important consideration in designing and/or replacing a natural joint with one or more implant components can be proper limb alignment or, when the malfunctioning joint contributes to a misalignment, proper realignment of the limb.

Certain embodiments described herein include collecting and using data from imaging tests to virtually determine in one or more planes one or more of an anatomic axis and a mechanical axis and the related misalignment of a patient's limb. The misalignment of a limb joint relative to the axis can identify the degree of deformity, for example, varus or valgus deformity in the coronal plane or genu antecurvatum or recurvatum deformity in the sagittal plane. Then, one or more of the patient-specific implant components and/or the implant procedure steps, such as bone resection, can be designed to help correct the misalignment.

The imaging tests that can be used to virtually determine a patient's axis and misalignment can include one or more of such as x-ray imaging, digital tomosynthesis, cone beam CT, non-spiral or spiral CT, non-isotropic or isotropic MRI, SPECT, PET, ultrasound, laser imaging, and photoacoustic imaging, including studies utilizing contrast agents. Data from these tests can be used to determine anatomic reference points or limb alignment, including alignment angles within the same and between different joints or to simulate normal limb alignment. Any anatomic features related to the misalignment can be selected and imaged. For example, in certain embodiments, such as for a knee or hip implant, the imaging test can include data from at least one of, or several of, a hip joint, knee joint and ankle joint. The imaging test can be obtained in lying, prone, supine or standing position. The imaging test can include only the target joint, or both the target joint and also selected data through one or more adjoining joints.

Using the image data, one or more mechanical or anatomical axes, angles, planes or combinations thereof can be determined. In certain embodiments, such axes, angles, and/or planes can include, or be derived from, one or more of a Whiteside's line, Blumensaat's line, transepicondylar line, femoral shaft axis, femoral neck axis, acetabular angle, lines tangent to the superior and inferior acetabular margin, lines tangent to the anterior or posterior acetabular margin, femoral shaft axis, tibial shaft axis, transmalleolar axis, posterior condylar line, tangent(s) to the trochlea of the knee joint, tangents to the medial or lateral patellar facet, lines tangent or perpendicular to the medial and lateral posterior condyles, lines tangent or perpendicular to a central weight-bearing zone of the medial and lateral femoral condyles, lines transecting the medial and lateral posterior condyles, for example through their respective centerpoints, lines tangent or perpendicular to the tibial tuberosity, lines vertical or at an angle to any of the aforementioned lines, and/or lines tangent to or intersecting the cortical bone of any bone adjacent to or enclosed in a joint. Moreover, estimating a mechanical axis, an angle, or plane also can be performed using image data obtained through two or more joints, such as the knee and ankle joint, for example, by using the femoral shaft axis and a centerpoint or other point in the ankle, such as a point between the malleoli.

As one example, if surgery of the knee or hip is contemplated, the imaging test can include acquiring data through at least one of, or several of, a hip joint, knee joint or ankle joint. As another example, if surgery of the knee joint is contemplated, a mechanical axis can be determined. For example, the centerpoint of the hip knee and ankle can be determined. By connecting the centerpoint of the hip with that of the ankle, a mechanical axis can be determined in the coronal plane. The position of the knee relative to said mechanical axis can be a reflection of the degree of varus or valgus deformity. The same determinations can be made in the sagittal plane, for example to determine the degree of genu antecurvatum or recurvatum. Similarly, any of these determinations can be made in any other desired planes, in two or three dimensions.

Various methods for virtually aligning a patient's lower extremity can be utilized, including various embodiments for determining a patient's tibial mechanical axis, femoral mechanical axis, and the sagittal and coronal planes for each axis as described herein. It should be understood that any current and future method for determining limb alignment and simulating normal knee alignment can be used. Once the proper alignment of the patient's extremity has been determined virtually, one or more surgical steps (e.g., resection cuts) may be planned and/or accomplished, which may include the use of surgical tools (e.g., tools to guide the resection cuts), and/or implant components (e.g., components having variable thicknesses to address misalignment).

### Modeling Articular Cartilage

Cartilage loss in one compartment can lead to progressive joint deformity. For example, cartilage loss in a medial compartment of the knee can lead to varus deformity. In certain embodiments, cartilage loss can be estimated in the affected compartments. The estimation of cartilage loss can be done using an ultrasound MRI or CT scan or other imaging modality, optionally with intravenous or intra-articular contrast. The estimation of cartilage loss can be as simple as measuring or estimating the amount of joint space loss seen on x-rays. For the latter, typically standing x-rays may be preferred. If cartilage loss is measured from x-rays using joint space loss, cartilage loss on one or two opposing articular surfaces can be estimated by, for example, dividing the measured or estimated joint space loss by two to reflect the cartilage loss on one articular surface. Other ratios or calculations are applicable depending on the joint or the location within the joint. Subsequently, a normal cartilage thickness can be virtually established on one or more articular surfaces by simulating normal cartilage thickness. In this manner, a normal or near normal cartilage surface can be derived. Normal cartilage thickness can be virtually simulated using a computer, for example, based on computer models, for example using the thickness of adjacent normal cartilage, cartilage in a contralateral joint, or other anatomic information including subchondral bone shape or other articular geometries. Cartilage models and estimates of cartilage thickness can also be derived from anatomic reference databases that can be matched, for example, to a patient's weight, sex, height, race, gender, or articular geometry(ies).

In certain embodiments, a patient's limb alignment can be virtually corrected by realigning the knee after establishing a normal cartilage thickness or shape in the affected compartment by moving the joint bodies, for example, femur and tibia, so that the opposing cartilage surfaces including any augmented or derived or virtual cartilage surface touch each other, typically in the preferred contact areas. These contact areas can be simulated for various degrees of flexion or extension.

### Deriving Implant Features

Once a patient's relevant anatomical features have been identified and/or assessed, and any desired models have been created and/or obtained, this information can be utilized by the automated system to design and/or select appropriate implant components, surgical procedures and associated surgical tools appropriate for treatment of the patient.

Various embodiments described herein relate to patient-adapted implants, guide tools, and related methods. Patient-adapted features can include patient-specific features and/or patient-engineered features. In various embodiments, patient-specific feature(s) of an implant component or guide tool can be achieved by analyzing imaging test data and selecting (e.g., preoperatively selecting from a library of implant components) the implant component that best fits one or more pre-determined patient-specific parameters that are derived from the imaging test. Moreover, an implant component or guide tool can include a patient-specific feature that is both selected and designed. For example, an implant component initially can be selected (e.g., preoperatively selected from a library of implants) to have a feature with a standard or blank dimension, or with a larger or smaller dimension than the predetermined patient-specific dimension. Then, the implant component can be machined (if selected from an actual library of implant components) or manufactured (if selected from a virtual library of implant components) so that the standard dimension or blank dimension or larger-dimensioned or smaller-dimensioned implant feature is altered to have the patient-specific dimension.

In addition or alternatively, certain embodiments relate to patient-engineered implants, guide tools, and related methods. Some embodiments relate to articular implant components having one or more patient-engineered features optimized from patient-specific data to meet one or more parameters to enhance one or more of the patient's biological features, such as one or more biological/anatomical structures, alignments, kinematics, and/or soft tissue impingements. Accordingly, the one or more patient-engineered features of an implant component can include, but are not limited to, one or more implant component surfaces, such as surface contours, angles or bone cuts, and dimensions such as thickness, width, depth, or length of one or more aspects of the implant component. The patient-engineered feature(s) of an implant component can be designed and/or manufactured (e.g., preoperatively designed and manufactured) based on patient-specific data to substantially enhance or improve one or more of the patient's anatomical and/or biological features. Methods for preparing certain patient-engineered features are described, for example, in U.S. Ser. No. 12/712,072, entitled “Automated Systems For Manufacturing Patient-Specific Orthopedic Implants And Instrumentation” filed Feb. 24, 2010, which is incorporated herein by reference.

As with the patient-specific feature(s) of an implant component or guide tool, the patient-engineered features of an implant component or guide tool can be designed (e.g., preoperatively designed and manufactured) or they can be selected, for example, by selecting an implant component that best meets the one or more predetermined parameters that enhance one or more features of the patient's biology. Moreover, an implant component or guide tool can include a patient-engineered feature that is both selected and designed. For example, an implant component initially can be selected (e.g., preoperatively selected from a library of implants) to have a feature with a larger or smaller dimension than the desired patient-engineered dimension. Then, the implant component can be machined (if selected from an actual library of implant components) or manufactured (if selected from a virtual library of implant components) so that the larger-dimensioned or smaller-dimensioned implant feature is altered to have the desired patient-engineered dimension.

In various embodiments, a single implant component, guide tool, and/or related method can include one or more patient-specific features, one or more patient-engineered features, and/or one or more standard (e.g., off-the-shelf features). The standard, off-the-shelf features can be selected to best fit with one or more of the patient-specific and/or patient-engineered features. For example, in a knee joint, a metal backed tibial component can include a standard locking mechanism and a patient-adapted (i.e., patient-specific or patient-engineered) perimeter of the tibial component. A patient-specific perimeter of the tibial component can be achieved, for example, by cutting the perimeter of a selected tibial component to match the patient's cortical bone perimeter in one or more dimensions of one more sections. Similarly, a polyethylene insert can be chosen that includes a standard locking mechanism, while the perimeter is adapted for better support to the patient's tibial bone perimeter or the perimeter of the metal backing.

In certain embodiments, implant components and/or related methods described herein can include a combination of patient-specific and patient-engineered features. For example, patient-specific data collected preoperatively can be used to engineer one or more optimized surgical cuts to the patient's bone and to design or select a corresponding implant component having or more bone-facing surfaces or facets (i.e., “bone cuts”) that specifically match one or more of the patient's resected bone surfaces. The surgical cuts to the patient's bone can be optimized (i.e., patient-engineered) to enhance one or more parameters, such as: (1) deformity correction and limb alignment (2) maximizing preservation of bone, cartilage, or ligaments, (3) maximizing preservation and/or optimization of other features of the patient's anatomy, such as trochlea and trochlear shape, (4) restoration and/or optimization of joint kinematics or biomechanics, (5) restoration or optimization of joint-line location and/or joint gap width, (6) and/or addressing one or more other parameters. Based on the optimized surgical cuts and, optionally, on other desired features of the implant component, the implant component's bone-facing surface can be designed or selected to, at least in part, negatively-match the shape of the patient's resected bone surface.

Any combination of one or more of parameters described herein and/or one or more additional parameters can be used in the design and/or selection of a patient-adapted (e.g., patient-specific and/or patient-engineered) implant component and, in certain embodiments, in the design and/or selection of corresponding patient-adapted resection cuts and/or patient-adapted guide tools. In particular assessments, a patient's biological features and feature measurements are used to select and/or design one or more implant component features and feature measurements, resection cut features and feature measurements, and/or guide tool features and feature measurements.

In certain embodiments, the assessment process includes selecting and/or designing one or more features and/or feature measurements of an implant component and, optionally, of a corresponding resection cut strategy and/or guide tool that is adapted (e.g., patient-adapted based on one or more of a particular patient's biological features and/or feature measurements) to achieve or address, at least in part, one or more of the following parameters for the particular patient: (1) correction of a joint deformity; (2) correction of a limb alignment deformity; (3) preservation of bone, cartilage, and/or ligaments at the joint; (4) preservation, restoration, or enhancement of one or more features of the patient's biology, for example, trochlea and trochlear shape; (5) preservation, restoration, or enhancement of joint kinematics, including, for example, ligament function and implant impingement; (6) preservation, restoration, or enhancement of the patient's joint-line location and/or joint gap width; and (7) preservation, restoration, or enhancement of other target features.

Correcting a joint deformity and/or a limb alignment deformity can include, for example, generating a virtual model of the patient's joint, limb, and/or other relevant biological structure(s); virtually correcting the deformity and/or aligning the limb; and selecting and/or designing one or more surgical steps (e.g., one or more resection cuts), one or more guide tools, and/or one or more implant components to physically perform and/or accommodate the correction.

Preserving, restoring, or enhancing bone, cartilage, and/or ligaments can include, for example, identifying diseased tissue from one or more images of the patient's joint, identifying a minimum implant thickness for the patient (based on, for example, femur and/or condyle size and patient weight); virtually assessing combinations of resection cuts and implant component features, such as variable implant thickness, bone cut numbers, bone cut angles, and/or bone cut orientations; identifying a combination of resection cuts and/or implant component features that, for example, remove diseased tissue and also provide maximum bone preservation (i.e., minimum amount of resected bone) and at least the minimum implant thickness for the particular patient; and selecting and/or designing one or more surgical steps (e.g., one or more resection cuts), one or more guide tools, and/or one or more implant components to provide the resection cuts and/or implant component features that provide removal of the diseased tissue, maximum bone preservation, and at least the minimum implant thickness for the particular patient.

Preserving or restoring one or more features of a patient's biology can include, for example, selecting and/or designing one or more surgical steps (e.g., one or more resection cuts), one or more guide tools, and/or one or more implant components so that one or more of the patient's postoperative implant features substantially match the patient's preoperative biological features or the patient's healthy biological features (e.g., as identified from a previous image of the patient's joint when it was healthy or from an image of the patient's contralateral healthy joint).

Enhancing one or more features of a patient's biology can include, for example, selecting and/or designing one or more surgical steps (e.g., one or more resection cuts), one or more guide tools, and/or one or more implant components so that the implant component, once implanted, includes features that approximate one or more features of a healthy biological feature for the particular patient.

Preservation or restoration of the patient's joint kinematics can include, for example, selecting and/or designing one or more surgical steps (e.g., one or more resection cuts), one or more guide tools, and/or one or more implant components so that the patient's post-operative joint kinematics substantially match the patient's pre-operative joint kinematics and/or substantially match the patient's healthy joint kinematics (e.g., as identified from previous images of the patient's joint when it was healthy or from an image of the patient's contralateral healthy joint).

Enhancing the patient's joint kinematics can include, for example, selecting and/or designing one or more surgical steps (e.g., one or more resection cuts), one or more guide tools, and/or one or more implant components that provide healthy joint kinematics estimated for the particular patient and/or that provide proper joint kinematics to the patient. Optimization of joint kinematics also can include optimizing ligament loading or ligament function during motion.

Preservation or restoration of the patient's joint-line location and/or joint gap width can include, for example, selecting and/or designing one or more surgical steps (e.g., one or more resection cuts), one or more guide tools, and/or one or more implant components so that the patient's joint-line and or joint-gap width substantially match the patient's existing joint-line and or joint-gap width or the patient's healthy joint-line and/or joint-gap width (e.g., as identified from previous images of the patient's joint when it was healthy or from an image of the patient's contralateral healthy joint).

Enhancing the patient's joint-line location and/or joint gap width can include, for example, selecting and/or designing one or more surgical steps (e.g., one or more resection cuts), one or more guide tools, and/or one or more implant components that provide a healthy joint-line location and/or joint gap width and/or estimated for the particular patient and/or that provide proper kinematics to the patient.

Exemplary patient-adapted (i.e., patient-specific and/or patient-engineered) features of various implant components described herein are identified in Table 2. One or more of these implant component features can be selected and/or designed based on patient-specific data, such as image data.

TABLE 2
Exemplary implant features that can be patient-adapted based
on patient-specific measurements

| Category | Exemplary feature |
|---|---|
| Implant or | One or more portions of, or all of, an external implant |
| implant or | component curvature |
| component | One or more portions of, or all of, an internal implant |
| (applies knee, | dimension |
| shoulder, hip, | One or more portions of, or all of, an internal or |
| ankle, or other | external implant angle |
| implant or | Portions or all of one or more of the ML, AP, SI |
| implant | dimension of the internal and external component and |
| component) | component features |
| | An locking mechanism dimension between a plastic or |
| | non-metallic insert and a metal backing component in |
| | one or more dimensions |
| | Component height |
| | Component profile |
| | Component 2D or 3D shape |
| | Component volume |
| | Composite implant height |
| | Component articular surface curvature |
| | Component bone-facing surface curvature |
| | Insert width |
| | Insert shape |
| | Insert length |
| | Insert height |
| | Insert profile |
| | Insert curvature |
| | Insert angle |
| | Distance between two curvatures or concavities |
| | Polyethylene or plastic width |
| | Polyethylene or plastic shape |
| | Polyethylene or plastic length |
| | Polyethylene or plastic height |
| | Polyethylene or plastic profile |
| | Polyethylene or plastic curvature |
| | Polyethylene or plastic angle |
| | Component stem width |
| | Component stem shape |
| | Component stem length |
| | Component stem height |
| | Component stem profile |
| | Component stem curvature |
| | Component stem position |
| | Component stem thickness |
| | Component stem angle |
| | Component peg width |
| | Component peg shape |
| | Component peg length |
| | Component peg height |
| | Component peg profile |
| | Component peg curvature |
| | Component peg position |
| | Component peg thickness |
| | Component peg angle |
| | Slope of an implant surface |
| | Number of sections, facets, or cuts on an implant |
| | surface |
| Femoral implant | Condylar distance of a femoral component, e.g., |
| or implant | between femoral condyles |
| component | A condylar coronal radius of a femoral component |
| | A condylar sagittal radius of a femoral component |
| Tibial implant | Slope of an implant surface |
| or implant | Condylar distance, e.g., between tibial joint-facing |
| component | surface concavities that engage femoral condyles |
| | Coronal curvature (e.g., one or more radii of curvature |
| | in the coronal plane) of one or both joint-facing |
| | surface concavities that engage each femoral condyle |
| | Sagittal curvature (e.g., one or more radii of curvature |
| | in the sagittal plane) of one or both joint-facing |
| | surface concavities that engage each femoral condyle |

The patient-adapted features described in Table 2 also can be applied to patient-adapted guide tools described herein.

The patient-adapted implant components and guide tools described herein can include any number of patient-specific features, patient-engineered features, and/or standard features. Illustrative combinations of patient-specific, patient-engineered, and standard features of an implant component are provided in Table 3. Specifically, the table illustrates an implant or implant component having at least thirteen different features. Each feature can be patient-specific (P), patient-engineered (PE), or standard (St). As shown, there are 105 unique combinations in which each of thirteen is either patient-specific, patient-engineered, or standard features.

TABLE 3
Exemplary combinations of patient-specific (P), patient-engineered
(PE), and standard (St) features¹ in an implant

| Implant | | | | | | | | | | | | | |
|---|---|---|---|---|---|---|---|---|---|---|---|---|---|
| system | Implant feature number² | | | | | | | | | | | | |
| number | 1 | 2 | 3 | 4 | 5 | 6 | 7 | 8 | 9 | 10 | 11 | 12 | 13 |
| 1 | P | P | P | P | P | P | P | P | P | P | P | P | P |
| 2 | PE | PE | PE | PE | PE | PE | PE | PE | PE | PE | PE | PE | PE |
| 3 | St | St | St | St | St | St | St | St | St | St | St | St | St |
| 4 | P | St | St | St | St | St | St | St | St | St | St | St | St |
| 5 | P | P | St | St | St | St | St | St | St | St | St | St | St |
| 6 | P | P | P | St | St | St | St | St | St | St | St | St | St |
| 7 | P | P | P | P | St | St | St | St | St | St | St | St | St |
| 8 | P | P | P | P | P | St | St | St | St | St | St | St | St |
| 9 | P | P | P | P | P | P | St | St | St | St | St | St | St |
| 10 | P | P | P | P | P | P | P | St | St | St | St | St | St |
| 11 | P | P | P | P | P | P | P | P | St | St | St | St | St |
| 12 | P | P | P | P | P | P | P | P | P | St | St | St | St |
| 13 | P | P | P | P | P | P | P | P | P | P | St | St | St |
| 14 | P | P | P | P | P | P | P | P | P | P | P | St | St |
| 15 | P | P | P | P | P | P | P | P | P | P | P | P | St |
| 16 | P | PE | PE | PE | PE | PE | PE | PE | PE | PE | PE | PE | PE |
| 17 | P | P | PE | PE | PE | PE | PE | PE | PE | PE | PE | PE | PE |
| 18 | P | P | P | PE | PE | PE | PE | PE | PE | PE | PE | PE | PE |
| 19 | P | P | P | P | PE | PE | PE | PE | PE | PE | PE | PE | PE |
| 20 | P | P | P | P | P | PE | PE | PE | PE | PE | PE | PE | PE |
| 21 | P | P | P | P | P | P | PE | PE | PE | PE | PE | PE | PE |
| 22 | P | P | P | P | P | P | P | PE | PE | PE | PE | PE | PE |
| 23 | P | P | P | P | P | P | P | P | PE | PE | PE | PE | PE |
| 24 | P | P | P | P | P | P | P | P | P | PE | PE | PE | PE |
| 25 | P | P | P | P | P | P | P | P | P | P | PE | PE | PE |
| 26 | P | P | P | P | P | P | P | P | P | P | P | PE | PE |
| 27 | P | P | P | P | P | P | P | P | P | P | P | P | PE |
| 28 | PE | St | St | St | St | St | St | St | St | St | St | St | St |
| 29 | PE | PE | St | St | St | St | St | St | St | St | St | St | St |
| 30 | PE | PE | PE | St | St | St | St | St | St | St | St | St | St |
| 31 | PE | PE | PE | PE | St | St | St | St | St | St | St | St | St |
| 32 | PE | PE | PE | PE | PE | St | St | St | St | St | St | St | St |
| 33 | PE | PE | PE | PE | PE | PE | St | St | St | St | St | St | St |
| 34 | PE | PE | PE | PE | PE | PE | PE | St | St | St | St | St | St |
| 35 | PE | PE | PE | PE | PE | PE | PE | PE | St | St | St | St | St |
| 36 | PE | PE | PE | PE | PE | PE | PE | PE | PE | St | St | St | St |
| 37 | PE | PE | PE | PE | PE | PE | PE | PE | PE | PE | St | St | St |
| 38 | PE | PE | PE | PE | PE | PE | PE | PE | PE | PE | PE | St | St |
| 39 | PE | PE | PE | PE | PE | PE | PE | PE | PE | PE | PE | PE | St |
| 40 | P | PE | St | St | St | St | St | St | St | St | St | St | St |
| 41 | P | PE | PE | St | St | St | St | St | St | St | St | St | St |
| 42 | P | PE | PE | PE | St | St | St | St | St | St | St | St | St |
| 43 | P | PE | PE | PE | PE | St | St | St | St | St | St | St | St |
| 44 | P | PE | PE | PE | PE | PE | St | St | St | St | St | St | St |
| 45 | P | PE | PE | PE | PE | PE | PE | St | St | St | St | St | St |
| 46 | P | PE | PE | PE | PE | PE | PE | PE | St | St | St | St | St |
| 47 | P | PE | PE | PE | PE | PE | PE | PE | PE | St | St | St | St |
| 48 | P | PE | PE | PE | PE | PE | PE | PE | PE | PE | St | St | St |
| 49 | P | PE | PE | PE | PE | PE | PE | PE | PE | PE | PE | St | St |
| 50 | P | PE | PE | PE | PE | PE | PE | PE | PE | PE | PE | PE | St |
| 51 | P | P | PE | St | St | St | St | St | St | St | St | St | St |
| 52 | P | P | PE | PE | St | St | St | St | St | St | St | St | St |
| 53 | P | P | PE | PE | PE | St | St | St | St | St | St | St | St |
| 54 | P | P | PE | PE | PE | PE | St | St | St | St | St | St | St |
| 55 | P | P | PE | PE | PE | PE | PE | St | St | St | St | St | St |
| 56 | P | P | PE | PE | PE | PE | PE | PE | St | St | St | St | St |
| 57 | P | P | PE | PE | PE | PE | PE | PE | PE | St | St | St | St |
| 58 | P | P | PE | PE | PE | PE | PE | PE | PE | PE | St | St | St |
| 59 | P | P | PE | PE | PE | PE | PE | PE | PE | PE | PE | St | St |
| 60 | P | P | PE | PE | PE | PE | PE | PE | PE | PE | PE | PE | St |
| 61 | P | P | P | PE | St | St | St | St | St | St | St | St | St |
| 62 | P | P | P | PE | PE | St | St | St | St | St | St | St | St |
| 63 | P | P | P | PE | PE | PE | St | St | St | St | St | St | St |
| 64 | P | P | P | PE | PE | PE | PE | St | St | St | St | St | St |
| 65 | P | P | P | PE | PE | PE | PE | PE | St | St | St | St | St |
| 66 | P | P | P | PE | PE | PE | PE | PE | PE | St | St | St | St |
| 67 | P | P | P | PE | PE | PE | PE | PE | PE | PE | St | St | St |
| 68 | P | P | P | PE | PE | PE | PE | PE | PE | PE | PE | St | St |
| 69 | P | P | P | PE | PE | PE | PE | PE | PE | PE | PE | PE | St |
| 70 | P | P | P | P | PE | St | St | St | St | St | St | St | St |
| 71 | P | P | P | P | PE | PE | St | St | St | St | St | St | St |
| 72 | P | P | P | P | PE | PE | PE | St | St | St | St | St | St |
| 73 | P | P | P | P | PE | PE | PE | PE | St | St | St | St | St |
| 74 | P | P | P | P | PE | PE | PE | PE | PE | St | St | St | St |
| 75 | P | P | P | P | PE | PE | PE | PE | PE | PE | St | St | St |
| 76 | P | P | P | P | PE | PE | PE | PE | PE | PE | PE | St | St |
| 77 | P | P | P | P | PE | PE | PE | PE | PE | PE | PE | PE | St |
| 78 | P | P | P | P | P | PE | St | St | St | St | St | St | St |
| 79 | P | P | P | P | P | PE | PE | St | St | St | St | St | St |
| 80 | P | P | P | P | P | PE | PE | PE | St | St | St | St | St |
| 81 | P | P | P | P | P | PE | PE | PE | PE | St | St | St | St |
| 82 | P | P | P | P | P | PE | PE | PE | PE | PE | St | St | St |
| 83 | P | P | P | P | P | PE | PE | PE | PE | PE | PE | St | St |
| 84 | P | P | P | P | P | PE | PE | PE | PE | PE | PE | PE | St |
| 85 | P | P | P | P | P | P | PE | St | St | St | St | St | St |
| 86 | P | P | P | P | P | P | PE | PE | St | St | St | St | St |
| 87 | P | P | P | P | P | P | PE | PE | PE | St | St | St | St |
| 88 | P | P | P | P | P | P | PE | PE | PE | PE | St | St | St |
| 89 | P | P | P | P | P | P | PE | PE | PE | PE | PE | St | St |
| 90 | P | P | P | P | P | P | PE | PE | PE | PE | PE | PE | St |
| 91 | P | P | P | P | P | P | P | PE | St | St | St | St | St |
| 92 | P | P | P | P | P | P | P | PE | PE | St | St | St | St |
| 93 | P | P | P | P | P | P | P | PE | PE | PE | St | St | St |
| 94 | P | P | P | P | P | P | P | PE | PE | PE | PE | St | St |
| 95 | P | P | P | P | P | P | P | PE | PE | PE | PE | PE | St |
| 96 | P | P | P | P | P | P | P | P | PE | St | St | St | St |
| 97 | P | P | P | P | P | P | P | P | PE | PE | St | St | St |
| 98 | P | P | P | P | P | P | P | P | PE | PE | PE | St | St |
| 99 | P | P | P | P | P | P | P | P | PE | PE | PE | PE | St |
| 100 | P | P | P | P | P | P | P | P | P | PE | St | St | St |
| 101 | P | P | P | P | P | P | P | P | P | PE | PE | St | St |
| 102 | P | P | P | P | P | P | P | P | P | PE | PE | PE | St |
| 103 | P | P | P | P | P | P | P | P | P | P | PE | St | St |
| 104 | P | P | P | P | P | P | P | P | P | P | PE | PE | St |
| 105 | P | P | P | P | P | P | P | P | P | P | P | PE | St |

¹S = standard, off-the-shelf, P = patient-specific, PE = patient-engineered (e.g., constant coronal curvature, derived from the patient's coronal curvatures along articular surface)
²Each of the thirteen numbered implant features represents a different exemplary implant feature, for example, for a knee implant the thirteen features can include: (1) femoral implant component M-L dimension, (2) femoral implant component A-P dimension, (3) femoral implant component bone cut, (4) femoral implant component sagittal curvature, (5) femoral implant component coronal curvature, (6) femoral implant component inter-condylar distance, (7) femoral implant component notch location/geometry, (8) tibial implant component M-L dimension, (9) tibial implant component A-P dimension, (10) tibial implant component insert inter-condylar distance, (11) tibial implant component insert lock, (12) tibial implant component metal backing lock, and (13) tibial implant component metal backing perimeter.

### Design/Select/Adapt Implant Bone-Facing Features and Surgical Cuts

In certain embodiments, the bone-facing surface of an implant can be designed to substantially negatively-match one more bone surfaces. For example, in certain embodiments at least a portion of the bone-facing surface of a patient-adapted implant component can be designed to substantially negatively-match the shape of subchondral bone, cortical bone, endosteal bone, and/or bone marrow. A portion of the implant also can be designed for resurfacing, for example, by negatively-matching portions of a bone-facing surface of the implant component to the subchondral bone or cartilage. Accordingly, in certain embodiments, the bone-facing surface of an implant component can include one or more portions designed to engage resurfaced bone, for example, by having a surface that negatively-matches uncut subchondral bone or cartilage, and one or more portions designed to engage cut bone, for example, by having a surface that negatively-matches a cut subchondral bone.

In various embodiments, the automated system will initially utilize the patient-specific anatomical information and/or unmodified models thereof in planning various surgical procedure steps, although the use of modified patient anatomical information and/or modified patient models thereof could be of use, alternatively as well as in combination with unmodified information/models. In certain embodiments, the bone-facing surface of an implant component includes multiple surfaces, also referred to herein as bone cuts. One or more of the bone cuts on the bone-facing surface of the implant component can be selected and/or designed to substantially negatively-match one or more surfaces of the patient's bone. The surface(s) of the patient's bone can include bone, cartilage, or other biological surfaces. For example, in certain embodiments, one or more of the bone cuts on the bone-facing surface of the implant component can be designed to substantially negatively-match (e.g., the number, depth, and/or angles of cut) one or more resected surfaces of the patient's bone. The bone-facing surface of the implant component can include any number of bone cuts, for example, two, three, four, less than five, five, more than five, six, seven, eight, nine or more bone cuts. In certain embodiments, the bone cuts of the implant component and/or the resection cuts to the patient's bone can include one or more facets on corresponding portions of an implant component. For example, the facets can be separated by a space or by a step cut connecting two corresponding facets that reside on parallel or non-parallel planes.

Designing Cuts to Preserve Bone, Cartilage and/or Soft Tissues

Traditional orthopedic implants incorporate bone cuts. These bone cuts achieve two objectives: they establish a shape of the bone that is adapted to the implant and they help achieve a normal or near normal axis alignment. For example, bone cuts can be used with a knee implant to correct an underlying varus of valgus deformity and to shape the articular surface of the bone to fit a standard, bone-facing surface of a traditional implant component. With a traditional implant, multiple bone cuts are placed. However, since traditional implants are manufactured off-the-shelf without use of patient-specific information, these bone cuts are generally pre-set for a given implant without taking into consideration the unique shape of the patient. Thus, by cutting the patient's bone to fit the traditional implant, more bone is often discarded than is necessary with an implant designed to address the particularly patient's structures and deficiencies.

In certain embodiments, resection cuts can be optimized to preserve the maximum amount of bone for each individual patient, based on a series of two-dimensional images or a three-dimensional representation of the patient's articular anatomy and geometry and the desired limb alignment and/or desired deformity correction. Resection cuts on two opposing articular surfaces can be optimized to achieve the minimum amount of bone resected from one or both articular surfaces.

By adapting resection cuts in the series of two-dimensional images or the three-dimensional representation on two opposing articular surfaces such as, for example, a femoral head and an acetabulum, one or both femoral condyle(s) and a tibial plateau, a trochlea and a patella, a glenoid and a humeral head, a talar dome and a tibial plafond, a distal humerus and a radial head and/or an ulna, or a radius and a scaphoid, certain embodiments allow for patient individualized, bone-preserving implant designs that can assist with proper ligament balancing and that can help avoid “overstuffing” of the joint, while achieving optimal bone preservation on one or more articular surfaces in each patient.

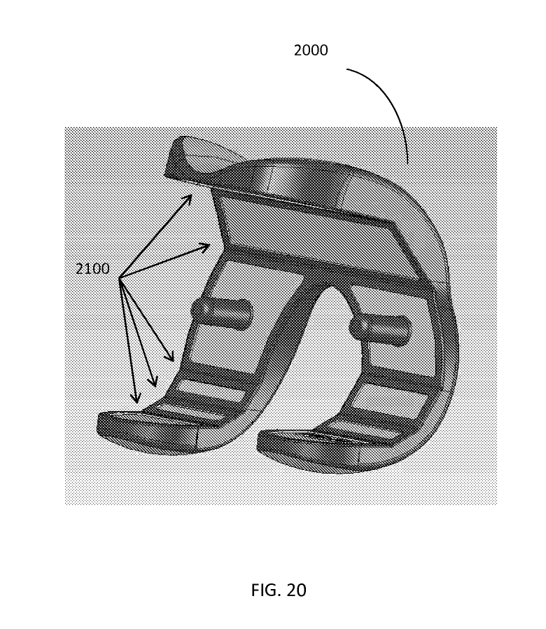

The resection cuts also can be designed to meet or exceed a certain minimum material thickness, for example, the minimum amount of thickness required to ensure biomechanical stability and durability of the implant. In certain embodiments, the limiting minimum implant thickness can be defined at the intersection of two adjoining bone cuts on the inner, bone-facing surface of an implant component. For example, in the femoral implant component 2000 shown in FIG. 20, the minimum thickness of the implant component appears at one or more intersections 2100. In certain embodiments of a femoral implant component, the minimum implant thickness can be less than 10 mm, less than 9 mm, less than 8 mm, less than 7 mm, and/or less than 6 mm.

In a knee, different resection cuts can be planned for a medial and lateral femoral condyle. In certain embodiments, a single bone cut can be optimized in a patient to maximize bone preservation in that select area, for example, a posterior condyle. Alternatively, multiple or all resection cuts can be optimized. Since a patient's medial and lateral femoral condyles typically have different geometries, including, for example, width, length and radii of curvature in multiple planes, for example, the coronal and the sagittal plane, then one or more resection cuts can be optimized in the femur individually for each condyle, resulting in resection cuts placed at a different depths, angles, and/or orientations in one condyle relative to the other condyle. For example, a horizontal cut in a medial condyle may be anatomically placed more inferior relative to the limb than a horizontal cut in a lateral condyle. The distance of the horizontal cut from the subchondral bone may be the same in each condyle or it can be different in each condyle. Chamfer cuts in the medial and lateral condyle may be placed in different planes rather than the same plane in order to optimize bone preservation. Moreover, chamfer cuts in the medial and lateral condyle may be placed at a different angle in order to maximize bone preservation. Posterior cuts may be placed in a different plane, parallel or non-parallel, in a medial and a lateral femoral condyle in order to maximize bone preservation. A medial condyle may include more bone cut facets than a lateral condyle in order to enhance bone preservation or vice versa.

In certain embodiments, a measure of bone preservation can include total volume of bone resected, volume of bone resected from one or more resection cuts, volume of bone resected to fit one or more implant component bone cuts, average thickness of bone resected, average thickness of bone resected from one or more resection cuts, average thickness of bone resected to fit one or more implant component bone cuts, maximum thickness of bone resected, maximum thickness of bone resected from one or more resection cuts, maximum thickness of bone resected to fit one or more implant component bone cuts.

In addition to optimizing bone preservation, another factor in determining the depth, number, and/or orientation of resection cuts and/or implant component bone cuts can be desired implant thickness. A minimum implant thickness can be included as part of the resection cut and/or bone cut design to ensure a threshold strength for the implant in the face of the stresses and forces associated with joint motion, such as standing, walking, and running. In various embodiments, a finite element analysis (FEA) assessment can be conducted on implant components, such as for femoral implant components of various sizes and with various bone cut numbers and orientations. The maximum principal stress observed in FEA analysis can be used to establish an acceptable minimum implant thickness for an implant component having a particular size and, optionally, for a particular patient (e.g., having a particular weight, age, activity level, etc). Before, during, and/or after establishing a minimum implant component thickness, the optimum depth of the resection cuts and the optimum number and orientation of the resection cuts and bone cuts, for example, for maximum bone preservation, can designed.

In certain embodiments, an implant component design or selection can depend, at least in part, on a threshold minimum implant component thickness. In turn, the threshold minimum implant component thickness can depend, at least in part, on patient-specific data, such as condylar width, femoral transepicondylar axis length, and/or the patient's specific weight. In this way, the threshold implant thickness, and/or any implant component feature, can be adapted to a particular patient based on a combination of patient-specific geometric data and on patient-specific anthropometric data.

In one embodiment, the automated system can calculate the closest location possible for resected surfaces and resected cuts relative to the articular surface of the uncut bone, e.g., so that all intersects of adjoining resected surfaces are just within the bone, rather than outside the articular surface. The software can move the cuts progressively closer to the articular surface. When all intersects of the resected cuts reach the endosteal bone level, the subchondral bone level, and/or an established threshold implant thickness, the maximum exterior placement of the resected surfaces is achieved and, with that, the maximum amount of bone preservation.

A weighting optionally can be applied to each bone with regard to the degree of bone preservation achieved. For example, if the maximum of bone preservation is desired on a tibia or a sub-segment of a tibia, femoral bone cuts can be adapted and moved accordingly to ensure proper implant alignment and ligament balancing. Conversely, if maximum bone preservation is desired on a femoral condyle, a tibial bone cut can be adjusted accordingly. If maximum bone preservation is desired on a patella, a resection cut on the opposing trochlea can be adjusted accordingly to ensure maximal patellar bone preservation without inducing any extension deficits. If maximum bone preservation is desired on a trochlea, a resection cut on the opposing patella can be adjusted accordingly to ensure maximal patellar bone preservation without inducing any extension deficits. Any combination is possible and different weightings can be applied. The weightings can be applied using mathematical models or, for example, data derived from patient reference databases.

Implant design and modeling also can be used to achieve ligament sparing, for example, with regard to the PCL and/or the ACL. An imaging test can be utilized to identify, for example, the origin and/or the insertion of the PCL and the ACL on the femur and tibia. The origin and the insertion can be identified by visualizing, for example, the ligaments directly, as is possible with MRI or spiral CT arthrography, or by visualizing bony landmarks known to be the origin or insertion of the ligament such as the medial and lateral tibial spines.

An implant system can then be selected or designed based on the image data so that, for example, the femoral component preserves the ACL and/or PCL origin, and the tibial component preserves the ACL and/or PCL attachment. The implant can be selected or designed so that bone cuts adjacent to the ACL or PCL attachment or origin do not weaken the bone to induce a potential fracture.

For ACL preservation, the implant can have two unicompartmental tibial components that can be selected or designed and placed using the image data. Alternatively, the implant can have an anterior bridge component. The width of the anterior bridge in AP dimension, its thickness in the superoinferior dimension or its length in mediolateral dimension can be selected or designed using the imaging data and, specifically, the known insertion of the ACL and/or PCL.

If desired, the posterior margin of an implant component, e.g., a polyethylene- or metal-backed tray with polyethylene inserts, can be selected and/or designed using the imaging data or shapes derived from the imaging data so that the implant component will not interfere with and stay clear of the PCL. This can be achieved, for example, by including concavities in the outline of the implant that are specifically designed or selected or adapted to avoid the ligament insertion. Any implant component can be selected and/or adapted in shape so that it stays clear of important ligament structures. Imaging data can help identify or derive shape or location information on such ligamentous structures. For example, the lateral femoral condyle of a unicompartmental, bicompartmental or total knee system can include a concavity or divot to avoid the popliteus tendon. Imaging data can be used to design a tibial component (all polyethylene or other plastic material or metal backed) that avoids the attachment of the anterior and/or posterior cruciate ligament; specifically, the contour of the implant can be shaped so that it will stay clear of these ligamentous structures. A safety margin, e.g., 2 mm or 3 mm or 5 mm or 7 mm or 10 mm can be applied to the design of the edge of the component to allow the surgeon more intraoperative flexibility. In a shoulder, the glenoid component can include a shape or concavity or divot to avoid a subscapularis tendon or a biceps tendon. In a hip, the femoral component can be selected or designed to avoid an iliopsoas or adductor tendons.

In certain embodiments, an implant component can include a fixed bearing design or a mobile bearing design. With a fixed bearing design, a platform of the implant component is fixed and does not rotate. However, with a mobile bearing design, the platform of the implant component is designed to rotate, e.g., in response to the dynamic forces and stresses on the joint during motion. Mobile bearing implants can comprise three components. A first component attached to a first articular surface, a second component attached to a second articular surface, and a third component slideably or rotatably or combinations thereof engageable between said first and second components. The third component or also mobile bearing can be entirely engineered. It can also include patient-adapted, patient-engineered or patient-specific features.

Moreover, the perimeter or shape of any mobile bearing component can be, at least partially, matched to the cortical bone, cartilage, subchondral bone, cut bone, one or more resection surfaces (including after grinding or milling), articular surface dimensions or shape including normal and diseased cartilage, or one or more implant components that include one or more patient specific or derived or patient engineered features as described through the application.

A rotating platform mobile bearing on the tibial implant component allows the implant to adjust and accommodate in an additional dimension during joint motion. However, the additional degree of motion can contribute to soft tissue impingement and dislocation. Mobile bearings are described elsewhere, for example, in U.S. Patent Application Publication No. 2007/0100462.

In certain embodiments, an implant can include a mobile-bearing implant that includes one or more patient-specific features, one or more patient-engineered features, and/or one or more standard features. For example, for a knee implant, the knee implant can include a femoral implant component having a patient-specific femoral intercondylar distance; a tibial component having standard mobile bearing and a patient-engineered perimeter based on the dimensions of the peripheral edge of the patient's cut tibia and allowing for rotation without significant extension beyond the perimeter of the patient's cut tibia; and a tibial insert or top surface that is patient-specific for at least the patient's intercondylar distance between the tibial insert dishes to accommodate the patient-specific femoral intercondylar distance of the femoral implant.

As another example, in certain embodiments a knee implant can include a femoral implant component that is patient-specific with respect to a particular patient's M-L dimension and standard with respect to the patient's femoral intercondylar distance; a tibial component having a standard mobile bearing and a patient-engineered perimeter based on the dimensions of the peripheral edge of the patient's cut tibia and allowing for rotation without significant extension beyond the perimeter of the patient's cut tibia; and a tibial insert or top surface that includes a standard intercondylar distance between the tibial insert dishes to accommodate the standard femoral intercondylar distance of the femoral implant.

### Exemplary Anterior Cut Method

In one exemplary method and rationale for performing an anterior bone cut on a targeted femur of a patient (in preparation for receiving a patient-specific or other type of implant), the anterior cut can be placed to satisfy one or more conditions and/or constraints, which can include: (1) placement relative to anterior cortex, e.g., to avoid notching, (2) angle with a selected biomechanical axis, (3) angle with a selected anatomical axis, (4) desired angle with a peg axis, (5) desired angle relative to a posterior cut, (6) angle with epicondylar axis or posterior condylar axis, (7) desired patellar coverage, (8) desired thickness of the implant, (9) desired thickness of bone resection (e.g., medial trochlear peak, lateral trochlear peak, trochlear groove), and (10) desired position relative to femoral shaft to avoid notching. If desired, the intended cut can be shifted along one, two, three or more degrees of freedom.

For example, a desired anterior cut can be placed by: (1) determining the flexion-extension angle to be divergent from the mechanical axis (e.g., in the sagittal plane), (2) determining the internal-external rotation to balance the resection of the medial and lateral trochlear surfaces, or alternatively, the lateral trochlear resection may be larger than the medial trochlear resection, and (3) determining the A-P position (depth) depth of cut plane to maximize patellar coverage without notching femoral cortex. In an alternative embodiment, the internal-external rotation and depth of the anterior cut can be determined to minimize implant thickness without compromising fatigue strength. In another embodiment, the anterior cut angle can be determined to maximize cement compression. In another embodiment, the anterior cut angle can be determined to facilitate implant insertion.

In various embodiments, the anterior cut can be used to “drive” the ultimate design of at least a portion of the implant, and because the implant profile is typically designed to satisfy different (often competing) criteria, the anterior cut can often be critical to proper implant design. For example, the implant profile can be shaped to optimize patellar tracking. The profile on the contralateral condyle can be shaped to optimize patellar coverage while also avoiding impingement with the contralateral meniscus/tibia, for example with a “teardrop” extension near the femoral notch that curves anteriorly away from the meniscal edge along the sulcus. The implant may also be tapered into the bone on the contralateral condyle to smoothen the transition between implant and articular surface for patellar tracking.

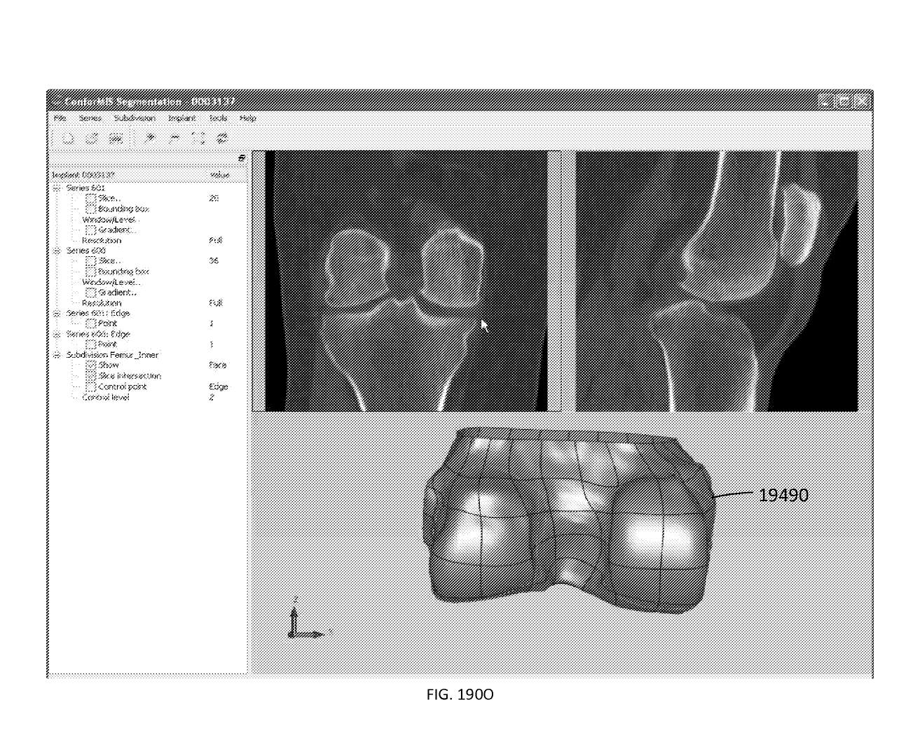

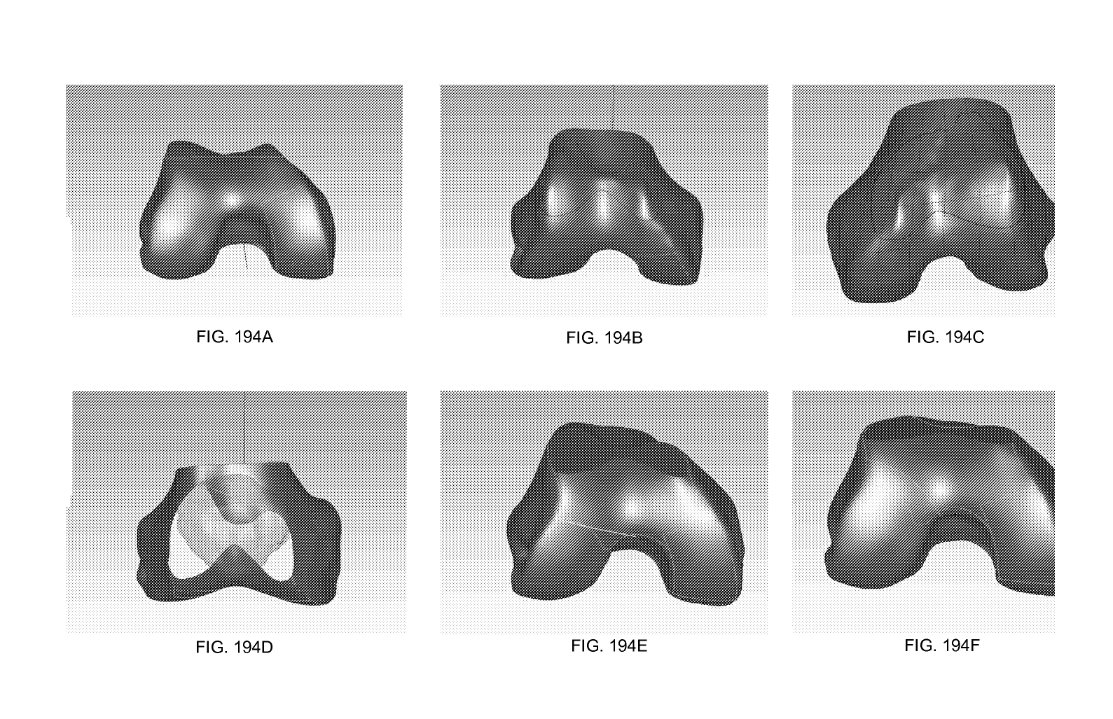

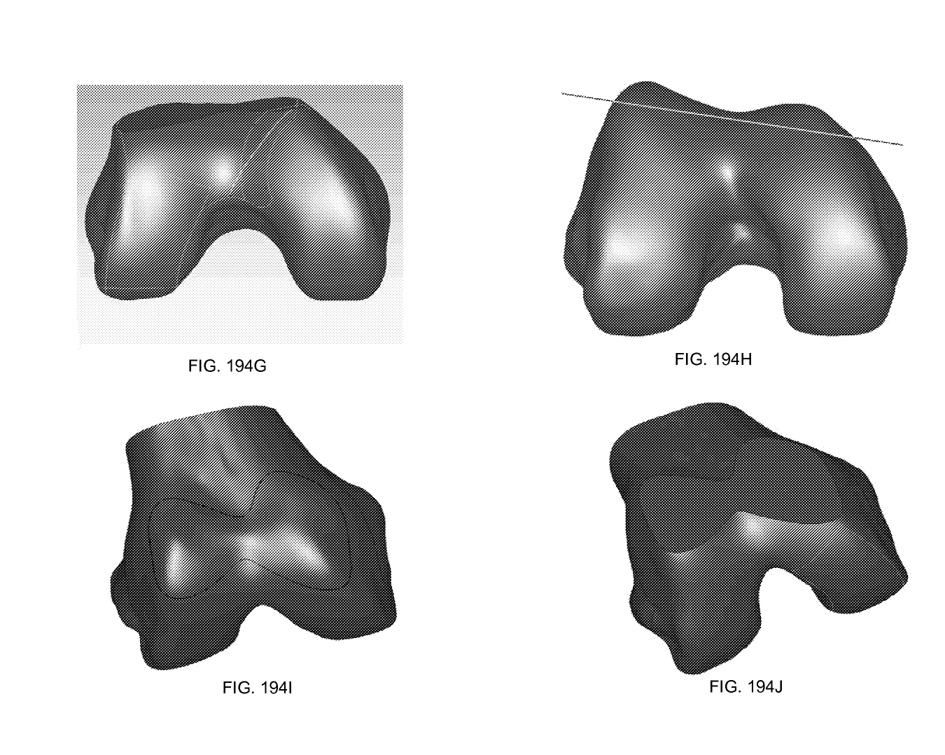

FIG. 194A depicts a first step of rotating the implant 2 degrees about the profile view x-axis to create the anterior cut. A default line appears on the model along with a set of three distance values listed in the top toolbar. These numbers represent the bone resection values in millimeters above the line to the highest point of the femur at the left, middle, and right of the femur in the current view. The middle value can be adjusted from 1.5 mm to 4 mm to achieve the best patella coverage. The automated and/or semi-automated program can aspire to minimize the resection by starting with the 1.5 mm value; the value will highlight in red if it falls below 1.5 mm. The line pivots at the center of the femur as the designer drags the line. The automated and/or semi-automated program can then pivot the line until the resection value on the lateral side is approximately 2 mm greater than the value on the medial side.

FIG. 194B depicts a preview of the cut, showing a desired characteristic “butterfly” shape. In this view the two sides of the condyle are connected and the lobes are not necessarily equal in size; the lateral lobe is generally larger.

FIG. 194C shows an example of where the anterior cut stretches too far beyond the anterior ridge of cartilage. In various embodiments, the automated and/or semi-automated program can reduce the central thickness of the cut to the minimum 1.5 mm, and if that does not bring the cut closer to the anterior ridge, increase the Profile View X-angle by increments of 1 degree and create the anterior cut again.

FIG. 194D shows a display of the patella surface in transparency mode. In various embodiments, the automated and/or semi-automated program's design goal can be to have a minimum ⅓ coverage area of the patella—or larger area if possible—with the smallest resection value. Looking normal to the anterior cut surface, the automated and/or semi-automated program may create a line linking the two sides of the anterior cut as shown below. The automated and/or semi-automated program can adjust the middle value sparingly on the Make Anterior Cut function to increase coverage.

FIG. 194E may assist the automated and/or semi-automated program by sketching the contra-lateral profile (“left” in this example) when the automated and/or semi-automated program displays the mesial border of the existing condyle sketch in the notch area. The sketch should hug the mesial edge of the notch within 1-2 mm of the visible edge. To create an arc as in the teardrop area, the automated and/or semi-automated program can select the startpoint of the arc, then continue the spline as an arc. The teardrop can fall below the sulcus to the “9:00” or the “3:00” position depending on the condyle. The automated and/or semi-automated program may desire to adjust the position of the teardrop in relation to the tibia: it should fall within the anterior rise of the spine. The teardrop is typically 5-6 mm in diameter and transitions to an approximate 15 mm diameter arc across the contra-lateral condyle and is capped by an approximate 4-5 mm diameter arc tangent to the sulcus on medial implants. On lateral implants, the final point of the arc on the contra-lateral sketch falls below the anterior cut and therefore below the sulcus. The automated and/or semi-automated program can finish the contra-lateral profile by crossing over the distal edge of the anterior cut and sketching within 2 mm of the side cut edge until the sketch crosses over the anterior cut.

FIGS. 194F and 194G show medial and lateral views, respectively, of a taper sketch outline that displays the starting location for the taper on the contra-lateral condyle. The automated and/or semi-automated program can follow the curvature of the implant sketch of the contra-lateral taper, offsetting the sketch 6-10 mm toward the trochlea; cross over both sides of the taper area as shown. The taper offset in lateral implants will fall in the narrower end of the 6-10 mm range and wrap on to the anterior cut surface

FIGS. 194H, 194I and 194J depict an operation of virtually cutting an anterior plane. The goal of the final steps is to define an anterior cutting plane. The program first displays the inner surface in profile view, then rotates it in the screen plane so that the common tangent to both condyles becomes horizontal, than rotates it 2 degrees around X-axis in the current view to provide divergence with posterior cutting plane. FIG. 194H depicts an anterior view. The automated and/or semi-automated program can modify the position and orientation of the cutting plane by moving the cutting line up and down (picking the line closer to its middle point) or by rotating it in the screen plane (picking it closer to its end point). For every position during this modification, the program can display the distances from the lowest horizon point and from two peak points.

FIG. 194I depicts the program displaying the cutting contour in 3d-mode. When the automated and/or semi-automated program accepts the position and orientation of the cutting plane, the program can virtually cut off the portion of the inner surface above the cutting plane and closes the hole with planar face. The result of this step, showing the inner surface and the flat anterior cut surface, is shown in FIG. 194J.

In certain embodiments, a model of at least part of a patient's joint can be used to directly generate a patient-engineered resection cut strategy for a surgical procedure. In certain embodiments, the model that includes at least a portion of the patient's joint also can include or display, as part of the model, one or more resection cuts, one or more drill holes, (e.g., on a model of the patient's femur), one or more guide tools, and/or one or more implant components that have been designed for the particular patient using the model. Moreover, one or more resection cuts, one or more drill holes, one or more guide tools, and/or one or more implant components can be modeled and selected and/or designed separate from a model of a particular patient's biological feature.

### Guide Tool Design

Various embodiments described herein include automated and/or semi-automated programs that design and/or select one or more guide tools having at least one patient-adapted bone-facing surface portion that substantially negatively-matches at least a portion of a biological surface at the patient's joint. The guide tool further can include at least one aperture for directing movement of a surgical instrument, for example, a securing pin or a cutting tool. One or more of the apertures can be designed to guide the surgical instrument to deliver a patient-optimized placement for, for example, a securing pin or resection cut. In addition or alternatively, one or more of the apertures can be designed to guide the surgical instrument to deliver a standard placement for, for example, a securing pin or resection cut. As used herein, “jig” also can refer to guide tools, which can include, for example, tools that guide resectioning of a patient's biological structure. Alternatively, certain guide tools can be used for purposes other than guiding a drill or cutting tool. For example, balancing and trial guide tools can be used to assess knee alignment and/or fit of one or more implant components or inserts.

Certain embodiments can include a guide tool that includes at least one patient-adapted bone-facing surface that substantially negatively-matches at least a portion of a biological surface at the patient's joint. The patient's biological surface can include cartilage, bone, tendon, and/or other biological surface. For example, in certain embodiments, patient-specific data such as imaging data of a patient's joint can be used to select and/or design (and/or create) an area on the articular surface that is free of articular cartilage. The area can be free of articular cartilage because it was never cartilage covered or because the overlying cartilage has been worn away or been removed. The imaging test can be specifically used to identify areas of full or near full thickness cartilage loss for designing the contact surface on the bone-facing surface of a patient-adapted guide tool. Alternatively, the area can be free of articular cartilage because an osteophyte has formed and is extending outside the cartilage. The guide tool then can rest directly on the bone, e.g., subchondral bone, marrow bone, endosteal bone or an osteophyte. By selecting and/or designing an area of the articular surface that is free of articular cartilage, it is possible to (a) reference the guide tool against the articular surface and (b) reference it against bone rather than cartilage.

In certain embodiments, patient-specific data such as imaging test data of a patient's joint can be used to identify a contact area on the articular surface for deriving an area on the bone-facing surface of a guide tool to substantially negatively-match the contact area on the subchondral bone surface. While the area may be covered by articular cartilage, the guide tool surface area can be specifically designed to match the subchondral bone contact area. The guide tool can have one or multiple areas that substantially negatively-match one or multiple contact areas on the subchondral bone surface. Intraoperatively, the surgeon or an automated robotic surgical apparatus can place the guide tool on the residual cartilage. Optionally, the surgeon or an automated robotic surgical apparatus can mark the approximate contact area on the cartilage and/or remove the overlying cartilage in the approximate contact area before replacing the guide tool directly onto the subchondral bone. In this manner, the surgeon or an automated robotic surgical apparatus can achieve more accurate placement of the guide tools that substantially negatively-matches subchondral bone.

In certain embodiments, patient-specific data such as imaging test data of a patient's joint can be used to identify a contact area on the articular surface for deriving an area on the bone-facing surface of a guide tool that substantially negatively-matches the endosteal bone or bone marrow contact area. While the area may be covered by articular cartilage, the guide tool surface area can be specifically designed to match the endosteal bone or bone marrow. The guide tool can have one or multiple areas that substantially negatively-match one or multiple areas on the endosteal bone or bone marrow. Intraoperatively, the surgeon or an automated robotic surgical apparatus can place the guide tool on the residual cartilage. Optionally, the surgeon or an automated robotic surgical apparatus can mark the approximate contact area on the cartilage and/orremove the overlying cartilage in the approximate contact area before replacing the guide tool directly onto the endosteal bone or bone marrow. In this manner, the surgeon or an automated robotic surgical apparatus can achieve more accurate placement of guide tools that match endosteal bone or bone marrow.

In certain embodiment, the articular surface or the margins of the articular surface can include one or more osteophytes. The guide tool can rest on the articular surface, e.g., on at least one of normal cartilage, diseased cartilage or subchondral bone, and it can include the shape of the osteophyte. In certain embodiments, patient-specific data such as imaging test data of a patient's joint can be used to derive an area on the bone-facing surface of the guide tool that substantially negatively-matches the patient's articular surface including the osteophyte. In this manner, the osteophyte can provide additional anatomic referencing for placing the guide tool. In certain embodiments, the osteophyte can be virtually removed from the joint on the 2D or 3D images and the contact surface of the guide tool can be derived based on the corrected surface without the osteophyte. In this setting, the surgeon or an automated robotic surgical apparatus can remove the osteophyte intraoperatively prior to placing the guide tool.

If a subchondral bone surface is used to assess the patient's biological surface, a standard cartilage thickness (e.g., 2 mm), or an approximate cartilage thickness derived from patient-specific data (e.g., age, joint-size, contralateral joint measurements, etc.) can be used as part of the design for the guide tool, for example, to design the size and bone-facing surface of the guide tool. The standard or approximate cartilage thickness can vary in thickness across the assessed surface area. In certain embodiments, this design can be used with a similarly designed implant, for example, an implant designed to include a standard or approximate cartilage thickness.

In certain embodiments, a model of at least part of a patient's joint can be used to directly generate a patient-adapted guide tool design for a surgical procedure. In certain embodiments, the model that includes at least a portion of the patient's joint also can include or display, as part of the model, one or more resection cuts, one or more drill holes (e.g., on a model of the patient's femur), one or more guide tools, and/or one or more implant components that have been designed for the particular patient using the model. Moreover, one or more resection cuts, one or more drill holes, one or more guide tools, and/or one or more implant components can be modeled and selected and/or designed separate from a model of a particular patient's biological feature.

### Guide Tool Configurations

The guide tools described herein can include any combination of patient-specific features, patient-engineered features, and/or standard features. For example, a patient-specific guide tool includes at least one feature that is preoperatively designed and/or selected to substantially match one or more of the patient's biological features. A patient-engineered guide tool includes at least one feature that is designed or selected based on patient-specific data to optimize one or more of the patient's biological features to meet one or more parameters, for example, as described elsewhere here, such as in Section 4. A standard guide tool includes at least one feature that is selected from among a family of limited options, for example, selected from among a family of 5, 6, 7, 8, 9, or 10 options. Accordingly, any one guide tool can be both patient-specific in view of its patient-specific features and patient-engineered in view of its patient-engineered features. Such a guide tool also can include standard features as well. Table 4 describes the various combinations of three features of a single guide tool with regard to being patient-specific features, patient-engineered features, and/or standard features of the exemplary guide tools. Moreover, in certain embodiments a set or kit of guide tools is provided in which certain guide tools in the set or kit include patient-specific, patient-engineered, and/or standard features. For example, a set or kit of guide tools can include any two or more guide tools described in Table 4.

TABLE 4
Patient-specific, patient-engineered, and standard
features of exemplary guide tools

| | Feature | Feature | Feature |
|---|---|---|---|
| Exemplary Guide tool | #1 | #2 | #3 |
| A guide tool that includes at least 3 PS | P | P | P |
| features | | | |
| A guide tool that includes at least 3 PE | PE | PE | PE |
| features | | | |
| A guide tool that includes at least 3 S | St | St | St |
| features | | | |
| A guide tool that includes at least 2 PS | P | P | PE |
| features and at least 1 PE feature | P | PE | P |
| | PE | P | P |
| A guide tool that includes at least 2 PS | P | P | St |
| features and at least 1 S feature | P | St | P |
| | St | P | P |
| A guide tool that includes at least 1PS | PE | PE | P |
| feature and at least 2 PE features | PE | P | PE |
| | P | PE | PE |
| A guide tool that includes at least 1PS | St | St | P |
| feature and at least 2 S feature | St | P | St |
| | P | St | St |
| A guide tool that includes at least 2PE | PE | PE | St |
| features and at least 1 S feature | PE | St | PE |
| | St | PE | PE |
| A guide tool that includes at least 1PE | St | St | PE |
| feature and at least 2 S features | St | PE | St |
| | PE | St | St |
| A guide tool that includes at least 1PS | P | PE | St |
| feature. at least 1 PE feature, and at least 1 | P | St | PE |
| S feature | PE | P | St |
| | PE | St | P |
| | St | P | PE |
| | St | PE | P |

P indicates a patient-specific feature,
PE indicates a patient-engineered feature, and
St indicates a standard feature

A guide tool can be used for one or more purposes during an implant procedure. For example, one or more guide tools can be used to establish resected holes in a patient's biological structure, to establish resected cuts in a patient's biological structure, and/or to balance or estimate fit of a joint implant.

### Guide Tool Markings

In certain embodiments, one or more guide tools described herein can include markings and/or electronically-detectable indicators to identify relevant features, for example, alignment indicators for anatomical and/or biomechanical axes. Such markings or indicators can intraoperatively guide a surgeon or an automated robotic surgical apparatus in the installation procedure. For example, the guide tool could include on its surface alignment indicators for the patient's Whiteside's AP trochlear line, the transepicondylar axis (TEA), the posterior condylar axis (PEA), a periphery of a tibial plateau or a femoral condyle (e.g., a cortical rim of the tibial surface). These indicators can be in colors and/or in raised geometries to strengthen the guide tool. Alternatively, these indicators may include a structural feature, for example, a groove on the guide tool surface that indicates a desired alignment with an anatomical feature of a patient such as for example the tibial periphery. Moreover, resection cut apertures on a guide tool can include numbers or other instructions to direct a surgeon or an automated robotic surgical apparatus in the proper resection procedure.

### Designing/Adapting/Selecting Implant Joint-Facing Features

In various embodiments described herein, the outer, joint-facing surface of an implant component includes one or more patient-adapted features (e.g., patient-specific and/or patient-engineered). For example, in certain embodiments, the joint-facing surface of an implant component can be designed to match the shape of the patient's biological structures. The joint-facing surface can include, for example, the bearing surface portion of the implant component that engages an opposing biological structure or implant component in the joint to facilitate typical movement of the joint. The patient's biological structure can include, for example, cartilage, bone, and/or one or more other biological structures.

In various embodiments, the automated system can initially utilize patient-specific anatomical information and/or unmodified models thereof in designing and/or selecting the joint-facing surface, or alternatively the system could utilize modified patient anatomical information and/or modified patient models thereof (as described herein) in designing and/or selecting the joint-facing features of the implant. In various alternative embodiments, combinations of such data sources may be used. In one exemplary embodiment, the joint-facing surface of an implant component is designed to match the shape of the patient's articular cartilage. For example, the joint-facing surface can substantially positively-match one or more features of the patient's existing cartilage surface and/or healthy cartilage surface and/or a calculated cartilage surface, on the articular surface that the component replaces. Alternatively, it can substantially negatively-match one or more features of the patient's existing cartilage surface and/or healthy cartilage surface and/or a calculated cartilage surface, on the opposing articular surface in the joint. As described below, corrections can be performed to the shape of diseased cartilage by designing surgical steps (and, optionally, patient-adapted surgical tools) to re-establish a normal or near normal cartilage shape that can then be incorporated into the shape of the joint-facing surface of the component. These corrections can be implemented and, optionally, tested in virtual two-dimensional and three-dimensional models. The corrections and testing can include kinematic analysis and/or surgical steps.

In certain embodiments, the joint-facing surface of an implant component can be designed to positively-match the shape of subchondral bone. For example, the joint-facing surface of an implant component can substantially positively-match one or more features of the patient's existing subchondral bone surface and/or healthy subchondral bone surface and/or a calculated subchondral bone surface, on the articular surface that the component attaches to on its bone-facing surface. Alternatively, it can substantially negatively-match one or more features of the patient's existing subchondral bone surface and/or healthy subchondral bone surface and/or a calculated subchondral bone surface, on the opposing articular surface in the joint. Corrections can be performed to the shape of subchondral bone to re-establish a normal or near normal articular shape that can be incorporated into the shape of the component's joint-facing surface. A standard thickness can be added to the joint-facing surface, for example, to reflect an average cartilage thickness. Alternatively, a variable thickness can be applied to the component. The variable thickness can be selected to reflect a patient's actual or healthy cartilage thickness, for example, as measured in the individual patient or selected from a standard reference database.

In certain embodiments, the joint-facing surface of a femoral implant component can be designed and/or selected to include one or more of a patient-specific curvature, at least in part, a patient-engineered curvature, at least in part, and a standard curvature, at least in part. Various exemplary combinations of implant components having patient-adapted (e.g., patient-specific or patient-engineered) and standard coronal and sagittal condylar curvatures are shown in Table 5.

TABLE 5
Exemplary combinations of patient-adapted and standard condylar curvatures
for a femoral implant component

| | Medial condyle | Medial condyle | Lateral condyle | Lateral condyle |
|---|---|---|---|---|
| Group | coronal | sagittal | coronal | sagittal |
| description | curvature | curvature | curvature | curvature |
| All | standard | standard | standard | standard |
| standard | | | | |
| curvatures | | | | |
| 1 patient- | patient-adapted | standard | standard | standard |
| adapted | standard | patient-adapted | standard | standard |
| curvature, | standard | standard | patient-adapted | standard |
| at least in part | standard | standard | standard | patient-adapted |
| 2 patient- | patient-adapted | patient-adapted | standard | standard |
| adapted | patient-adapted | standard | patient-adapted | standard |
| curvatures, | patient-adapted | standard | standard | patient-adapted |
| at least in | standard | patient-adapted | patient-adapted | standard |
| part | standard | patient-adapted | standard | patient-adapted |
| | standard | patient-adapted | standard | patient-adapted |
| | standard | standard | patient-adapted | patient-adapted |
| 3 patient- | patient-adapted | patient-adapted | patient-adapted | standard |
| adapted | patient-adapted | patient-adapted | standard | patient-adapted |
| curvatures, | patient-adapted | standard | patient-adapted | patient-adapted |
| at least in part | standard | patient-adapted | patient-adapted | patient-adapted |
| 4 patient- | patient-adapted | patient-adapted | patient-adapted | patient-adapted |
| adapted curvatures, | | | | |
| at least in part | | | | |

In certain embodiments, the joint-facing surface of the femoral implant component can be designed and/or selected to include a patient-specific curvature, at least in part. For example, any one or more of a coronal curvature of the medial condyle, a sagittal curvature of the medial condyle, a coronal curvature of the lateral condyle, and a sagittal curvature of the lateral condyle can be designed and/or selected preoperatively to substantially match the patient's corresponding curvature, e.g., subchondral bone or cartilage, at least in part, or can be derived from the patient's corresponding curvature, e.g., of subchondral bone or cartilage, at least in part. Portions or all of the sagittal curvature on a medial and/or lateral condyle can also be engineered. Portions or all of the coronal curvature on a medial and/or lateral condyle can also be engineered. Thus, engineered surface portions can be present in the same plane concomitant with patient adapted or derived curvatures.

The above embodiments are not only applicable to knee implants, but are applicable to implants in other parts of the body, e.g., an acetabulum, a femoral head, a glenoid, a humeral head, an elbow joint, a wrist joint, an ankle joint, a spine, etc.

### Approximation of Patient-Specific Features

In various exemplary embodiments, the design and/or selection of bone-facing and joint facing surfaces can include the processing of patient anatomical data and/or modeling data by automated systems to approximate various patient-specific implant component features. For example, implant components may be designed and/or selected to approximate one or more articular curvatures of a specific patient using image data/models of that patient. This may be accomplished in several ways.

For example, a library of designs can be stored electronically that has various curvature shapes that are best fit to the curvature of a specific patient, based, for example, on image data of the patient's bone. The curvature may be one or more curvatures associated with the joint, such as the curvatures of the articular surfaces. For example, the curvature could be a J-curve of one or both of the patient's femoral condyles as imaged in the sagittal plane or the curvature of one or both articular surfaces of the condyles imaged in a coronal plane.

In addition to approximating these curvatures directly from the image data, these curvatures can be approximated indirectly, using measurements of the patient's joint and selecting an implant design or an actual implant from a set of pre-existing implant designs and/or actual implants. For example, a set of measurements, such as condyle length, width and/or height can be used in various combinations to create an implant that approximates an outer curvature by creating a database of curvatures that typically correspond to various combinations of patient specific joint measurements.

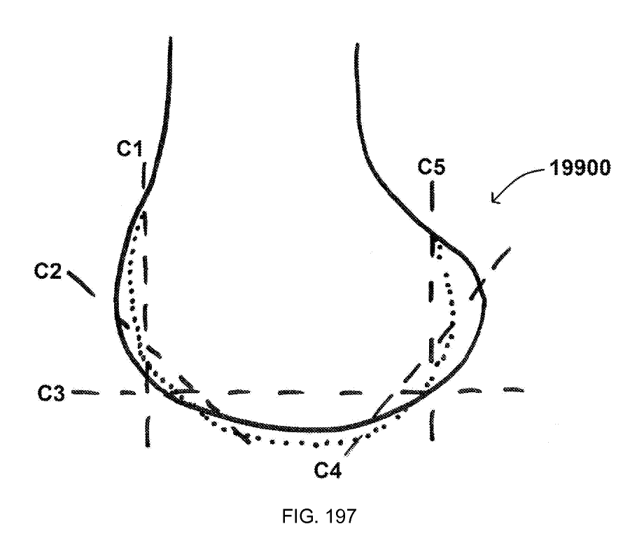

Additionally, an implant or implant design can be selected to approximate an outer curvature of the patient's joint based the location of the faceted cuts to be made to the joint surface during surgery. For example, a femoral implant for a patient's knee joint can be selected from a set of pre-existing implant designs based on the individualized placement of bone cuts on the patient's bone during the initial planning stages of the implant design and/or surgery. Referring to FIG. 197, using images of the patient's knee, a 3-dimensional virtual model of the patient's distal femur 19900 can be created. Virtual bone cuts (for example, cuts C1-C5 shown in FIG. 197) may be placed on the 3-D bone model. These may be placed, for example, based on predetermined design rules. The design rules can provide, for example, an optimized placement to minimize the amount of bone resection, biomechanical alignment of the implant, structural alignment of the implant, deepest cut depth, desired or minimum or maximum angles between cut planes and/or other desired design goals. The placed bone cut surfaces can then be compared to the candidate implant designs, for example using a best fit analysis.

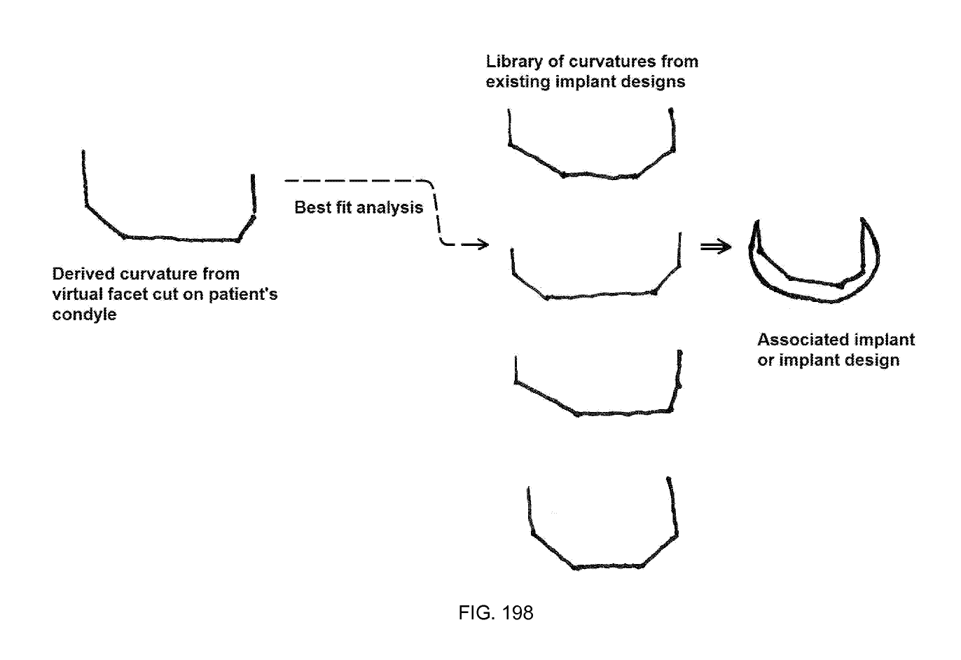

An exemplary best fit process is illustrated in FIG. 198. In this example, a curvature extracted following the virtual placement of bone cuts, such as in FIG. 197, is then compared to a library of curvatures from a set of pre-existing implant designs. The implant designs can be a set of any number of implant designs from a few, as shown in the figure for simplicity, to a large array of variations which would preferably be used to provide a large number of possible outer curvatures from which to select to better and more precisely approximate the outer articular curvature of the patient. The pre-existing implant designs can be created, for example, based on similar design principals as used to place the cuts, thereby creating a set of implant designs (or actual implants) that have similar outer curvatures, such as the articular curvatures of a condyle, to that of the patient's bone. Thus, when the best fit of the inner curvature is determined and an implant design (or actual implant) is selected from the library of implant designs (or actual implants), the selected implant design(s) (or actual implant(s)) can also approximate the corresponding outer articular curvature of the condyle of the patient. Thus, the resulting implant that is used during surgery (as selected, manufactured, or manufactured after further alteration of the physical implant and/or the pre-existing implant design) can closely correspond to and approximate the natural, derived and/or intended articular curvature of the patient.

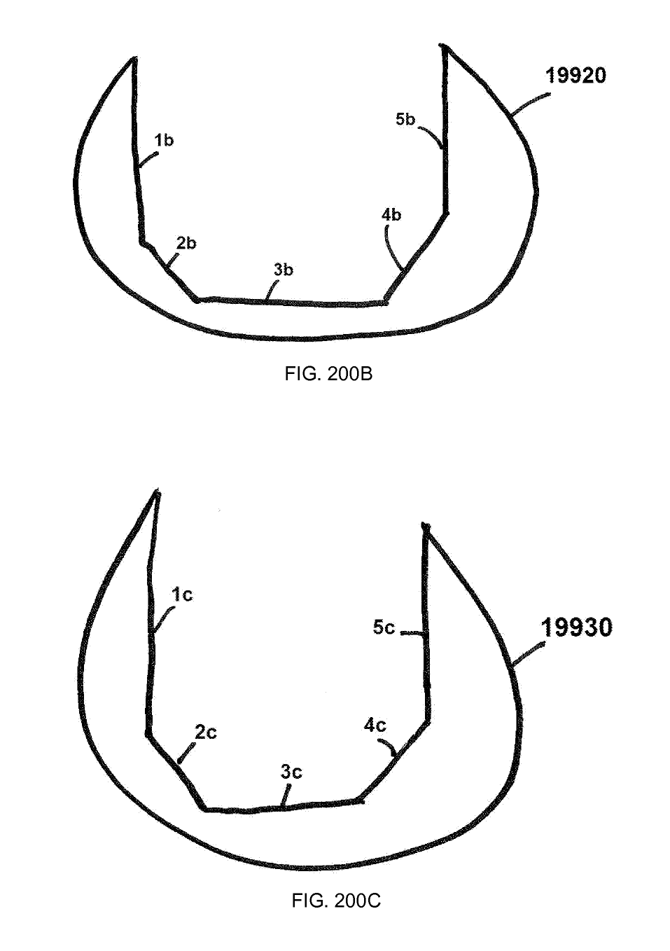

By selecting, for example, an implant from a set of pre-existing implant designs, such as those shown in FIGS. 200A through 200C, a close approximation of the outer articular surface of the patient's condyle can be achieved. For example, if the “best fit” implant selected was the implant shown in FIG. 200B, an implant incorporating bone cuts having a good fit with the patient's femur would desirably be selected, desirably also resulting in a good fit for the remaining articular surface (if any) as well as compared to the original articular surface. In contrast, if the implant designs shown in FIG. 200A or 200C were selected over that of FIG. 200B, the bone cuts shown would have a poorer fit with the patient's femur, likely also resulting in a poorer fit of the articular surface. Thus, the selection of fit optimized bone cuts can help in also achieving a good or better fit of the implant to the native, uncut articular surface.

Many variations of the above example are possible. For example, the curves can be in one or more planes, such as sagittal or coronal. The curves can be articular and/or other curves of the joint. The curves can extend along the entire length of the joint or can be a portion of the joint. The curves can be combined using single designs or by combining designs. For example, the j-curves of both medial and lateral condyles of a knee joint of a patient can be approximated using virtual cuts as described above, and a best fit of a single implant design can be undertaken to select one implant design (or actual implant) using the faceted virtual curves of both condyles, or, alternatively, each faceted virtual curve can be analyzed to determine a best fit for each condyle. In the latter case, two implant designs can be selected (one medial, and one lateral) and combined during subsequent implant design processes.

In another example, the faceted cuts can also be fixed in shape and position, and they can be taken from a template. Thus, instead of comparing existing designs to the virtual individual cuts placed on the model of the patient's joint, the faceted planes on the inner articular surface of the existing implant designs can be virtually compared to the bone model (or other model or image of the joint) to select a best fit directly from the templates. In a similar way, the methods presented here may be used to select a pre-manufactured implant from a set of existing implants.

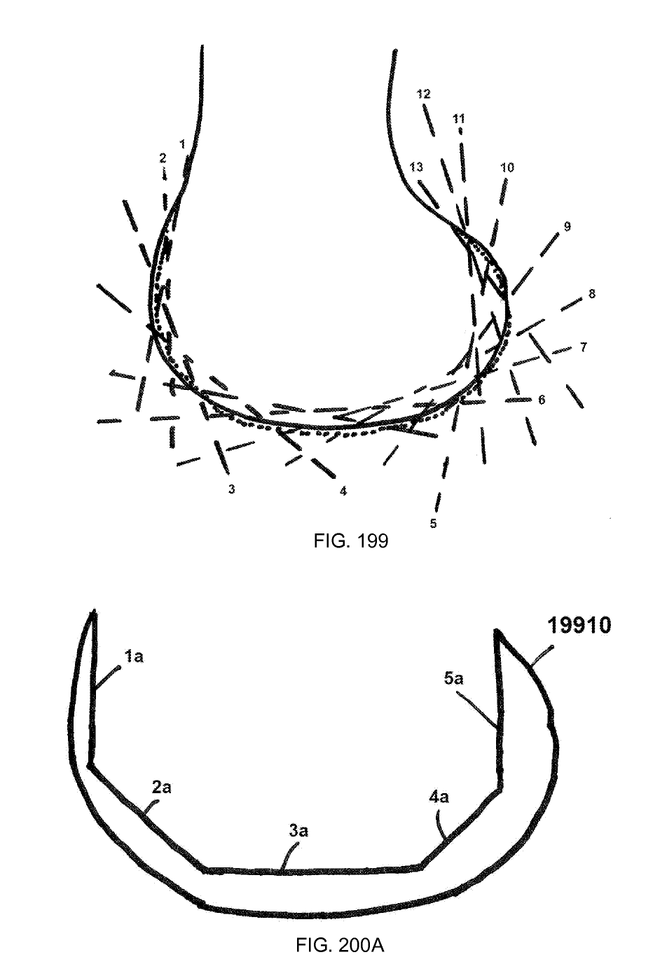

This method can be applicable to knee implants with traditional 5 cuts, for example an anterior, posterior, distal, anterior chamfer and posterior chamfer cut. It can also be used for femoral knee implants with more cuts, for example 7, 10, 13 or 15 cuts, with an example of an exemplary 13-cut plan shown in FIG. 199. Alternatively, however, more cuts may allow for better minimization of bone resection.

The cuts can be used to indirectly derive information on the patient's articular surface. For example, the edges or points on the edges between adjacent cuts can be used to approximate or describe the curvature of the articular surface. The higher the number of cuts and thus the number of edges between adjacent cuts, the more precisely the shape or curvature of the articular surface the method is likely to approximate. This technique allows for selecting an implant that fits the patient's articular surface geometry without assessing the shape or curvature of the articular surface directly. The more bone cuts are used, the more accurately the estimation of the articular surface or curvature or shape should be. Similarly, the use of additional bone cuts at areas of high curvature assist in more accurate approximation of the articular surface and curvature, with a lower number of cuts required at more planar (and/or less curved) locations. Bone cuts can be rotated around a single or multiple axis, parallel and non-parallel. In this manner, a true 3D shape of a joint can be described. Thus, a series of 2D planes or planar objects can be used to estimate or derive an articular curvature or shape, a curvature or shape of articular cartilage, or a curvature or shape of subchondral bone. The estimation of the shape of the articular surface using 13 bone cuts results in asimilarity between articular curvature (solid) and estimated articular curvature (stippled). The more bone cuts are used, the more accurate the estimation of the articular surface or curvature or shape. In this manner, a true 3D shape of a joint can be described

In a variation of the above example, a higher number of virtual cuts may be used to select the most suitable articular surface of an implant, while a lower number of virtual cuts, e.g., the 5 traditional cuts or another number such as 1, 2 3, 4, 6, 7, 8, 9 or 10, is used to determine the inner surface of the implant. After one or more of the shapes and/or curvatures have been derived in this manner, the number of cuts can optionally be reduced for the actual physical implant to be selected or designed. For example, if 20 cuts were used to derive or approximate a shape or curvature, the number of virtual cuts can subsequently be reduced, for example, to a total of five, which is the standard number of facet cuts currently used on standard, non-patient specific and non-patient matched implants. This may be done, for example, by finding the average position of 4 consecutive cuts to select or design the implant. Similarly, the process can simply select a predetermined set of cuts to form the five cut curvature, such as the first cut, last cut and every fourth or fifth cut between the first and last cuts. When fewer cuts are selected, the location, orientation, or depth of the cuts can be adjusted, for example, to account for the increased volume of bone that may need to be resected using fewer cuts. The five or other number bone cuts can compared to a fixed template and compared to determine a best fit, similar to the methods discussed above. In both cases, the outer articular curvatures of the pre-existing implant designs (or actual implants) can be predetermined using design rules and/or parameters that result in a similar outer curvature of the patient's joint. Of course, bone cuts can be combined with implant inner surfaces that conform to the existing bony and/or articular surface anatomy, if desired.

In alternative variations, a best-fit analysis, shortest point analysis, smoothing function or other methods may be used alone or in conjunction with other parameters such as width, height, curvature of the medial and/or lateral condyle or curvature of the notch to select the most suitable implant design. The same processes and methodologies can be applied to other joints, e.g., the hip or shoulder, where a series of cuts can be used to estimate or derive a shape or curvature, and wherein the shape or curvature information can be improved by combining it with information about other anatomic features.

The methods described herein can also be used to create implant designs and/or actual implants. Alternatively, these techniques can be used to generate a design for an implant to be manufactured that uses different modules. One module can be used to select the articular surface from a set of articular surface modules. This is combined with a suitable internal surface component from a set of modules, so that the internal surface and articular surface are combined to result in a virtual model of the implant that can be manufactured using, for example, just-in-time manufacturing techniques. Further modules may optionally refine the implant design based on various requirements, including material type and availability, cost, manufacturability, manufacturing time, implant strength and/or material maximum and minimum allowable implant thicknesses.

In another embodiment, the technique described above can be used to design a complete implant for the specific patient. For example, reference points on the simulated cuts can be used to determine a J-curve or curvature or set of J-curves or curvatures fitted to the individual patient. From the J-curves or curvatures, the articular surface of the implant is constructed and combined with the cut surfaces, which build the internal surface of the implant.

Alternatively, the foregoing embodiments can be used to select an implant from a library of pre-existing implant components. Optionally, the implant can then be modified by adding or subtracting material, e.g., by CNC operations or milling operations.

Single or multi-component implants can be used. For example, in a knee, an inner component can include fixed bone cuts, e.g., 5 fixed bone cuts on a femoral component. An outer component can be attachable to an inner component, wherein the outer component can have a patient-adapted shape. The outer component can have an optional locking mechanism that combines the outer component with the inner component, e.g., in a femur or tibia.

The articular surface of the implant can have various digital representations, like any other organically shaped surface, including, for example, a polygonal mesh, a parametric surface or an implicit surface. A parametric surface can, for example, be formulated as a B-spline or a subdivision surface. Implicit surfaces can be defined, without limitation, as level sets, isosurfaces, or adaptive data structures. Alternatively, the surface can be represented volumetrically or as a point cloud.

The articular implant surface can, for example, be constructed from the bone cuts as follows:

First, points on the intersection lines between two adjacent bone cuts are determined, for example the center points of each intersection line. A guide curve approximating or interpolating these points is then computed. The guide curve describes the surface shape in one direction. A set of support curves is calculated at an angle to the guide curve, for example at a perpendicular angle. These support curves describe the surface shape in the other direction and can have a fixed shape, for example an arc of a fixed radius or an elliptical segment, or a variable shape. The surface can finally be calculated, from the guide curve and the support curves, for example using a loft surface operation.

Several guide curves and corresponding sets of support curves can be combined into a single surface to form a more complex surface definition.

### Anatomical/Implant Modeling, Selection and Adaptation

As previously described, a femoral implant for a patient's knee joint can be selected from a set of pre-existing implant designs using a derived virtual placement of bone cuts. Using image data from the patient's knee, a 3-dimensional virtual model of the patient's distal femur is created. Virtual bone cuts are placed on the 3-D bone model. These bone cuts can optionally be optimized to minimize the amount of bone resection for a given number of cuts, or the number of virtual cuts can vary depending upon the physician's preference and/or user-defined features programmed into an automated system.

In one embodiment, the derived virtual placement of bone cuts in implant selection can be performed utilizing a traditional 5 cut architecture applicable to knee implants. Exemplary 5-cut placement can comprise, for example, an anterior, posterior, distal, anterior chamfer and posterior chamfer cuts. Various other embodiments can utilize more cuts for the femoral knee implants analysis, for example 7, 10 or 15 cuts. More cuts will desirably allow for better minimization of bone resection.

In other embodiments, the virtually placed bone cuts can be further (or alternatively) used to indirectly derive information on the patient's articular surface. For example, the edges or points on the edges between adjacent cuts (such as, for example, the edge along the junction of the anterior and anterior chamfer bone cuts) can be used to approximate and or describe the curvature of the articular surface. Depending upon the modeling and distribution of the cuts on the virtual model, a higher the number of cuts (and corresponding higher number edges between adjacent cuts), the more precisely or accurately the shape or curvature of the articular surface can be approximated. If desired, the virtual model can base the number and/or distribution of virtual cut surfaces upon geometric or anatomical features or constraints, such as, for example, to avoid a bone cut having a depth of over 2.5 mm, or possibly avoid having adjacent cut bone surfaces separated by a given angle (i.e., avoid acute or obtuse angles, or possibly limit acceptable adjacent surface angles to less than approximately 30, 45 or 60 degrees). Similarly, the method could estimate the volume of bone removal per virtual bone cut and limit or exceed this amount for some virtual cuts and not for others. The derived articular surface could further include a “thickening” or offset feature to accommodate an estimated depth of virtual articular cartilage, such as, for example, an offset or 1 to 3 mm from the derived surface, in calculating the final derived articular surface.

By modeling the virtual articular surface in one or more of the methods described herein, the present embodiments facilitate and enable the selection of implant(s) that fit (or highly approximate) the patient's articular surface geometry without assessing the shape or curvature of the articular surface directly. Depending upon the method chosen, the greater number of virtual bone cuts used, the more accurate the estimation of the articular surface or curvature or shape. Virtual bone cuts can be rotated around a single or multiple axis, including parallel and non-parallel. In this manner, a true 3D shape of a joint can be derived and/or described. In various embodiments, a series of 2D planes or planar objects can be used to estimate or derive an articular curvature or shape, a curvature or shape of articular cartilage, or a curvature or shape of subchondral bone.

As previously noted, after the shape and/or curvature have been derived using virtual bone cuts in the manner described above, the number of virtual bone cuts can optionally be reduced or increased, as well as the cuts themselves rearranged and/or resized in any manner, in preparation for deriving and/or modeling the actual physical implant to be selected or designed. For example, if 20 virtual bone cuts were used to derive a shape or curvature, these virtual bone cuts could subsequently be reduced to 5 (e.g., by finding the average position of 4 consecutive cuts or by various other means) to model, select and/or design the implant. The number of virtual bone cuts could similarly be increased. If desired, the five or other number bone cuts could be a fixed amount, shape, size and/or orientation, with the implant selected based on a best fit to the derived/estimated outer articular curvature/shape. Alternatively, the five bone cuts could be selected to best match the estimated outer articular curvature/shape derived from the higher number of cuts prior to “down-sampling” of the number of cuts, with the virtual outer implant surface retained as part of the final implant design.

In various alternative embodiments, the number of bone cuts and the analyses conducted herein, could be varied to “best suit” the given feature to be modeled. For example, a larger number of virtual bone cuts, or increasing the number or distribution of virtual bone cuts in certain areas of the femur may create a highly accurate virtual model of the outer articular curvature, but a lower number of virtual bone cuts may be more appropriate for modeling the inner bone-facing surface of the implant. The use of appropriate distributions and numbers of virtual bone cuts, therefore, can improve the accuracy of modeling and eventual design, selection and/or manufacture of appropriate implant(s) for the patient.

For example, a higher number of virtual cuts may be used to select the most suitable articular surface of an implant, while a lower number of virtual cuts, e.g., the 5 traditional cuts or another number such as 6, is used to determine the inner surface of the implant. This combination may be used to select the best fitting implant from a set of pre-manufactured implants. This method can also be used to design an implant. Alternatively, this technique can be used to generate a design for an implant to be manufactured that uses different modules. One module can be used to select the articular surface from a set of articular surface modules. This is combined with a suitable internal surface component from a set of modules, so that the internal surface and articular surface are combined to result in a virtual model of the implant that can be manufactured using, for example, just-in-time manufacturing techniques.

If desired, a “best-fit” analysis, shortest point analysis, smoothing function(s) or other methods (known in the art or developed in the future) of the cuts may be used alone or in conjunction with other parameters such as bone width, height, curvature of the medial and/or lateral condyle or curvature of the notch to select the most suitable implant design. Similarly, implant width, height, thickness, curvature of the inner bone-facing and/or outer medial and/or lateral derived condylar surface(s) and/or curvature of the implant notch may also be considered in selecting the most suitable implant design. Moreover, certain implant characteristics may necessitate a re-sampling or re-derivation of inner or outer surfaces, such as, for example, a desired minimum implant thickness to accommodate static and/or repetitive (fatigue and/or wear) loading of the implant. Where FEA (finite element analysis) or other analyses indicate the implant is undesirably thin in one location, a repositioning (or reangulation) of a virtual inner bone cut plane may result in thickening of the area of interest in the implant, alleviating further concern without requiring an overall increase in the implant thickness or significant increase in sacrifice of bony support tissues. Similar modules may troubleshoot other implant factors, such as material types, strength, wear and machineability concerns.

In other embodiments, the techniques described herein can be utilized to model and design a complete implant for the specific patient. For example, reference points on the simulated cuts can be used to determine a J-curve or curvature or set of J-curves or curvatures fitted to the individual patient. From the J-curves or curvatures, the articular surface of the implant is constructed and combined with the cut surfaces, which build the internal surface of the implant. Alternatively, the various methods described herein can be utilized to select an implant from a library of pre-existing implant components. Optionally, the implant can then be modified by adding or subtracting material, e.g., by CNC operations or milling operations.

In various embodiments, single or multi-component implants can be modeling, designed, selected and/or manufactured. For example, in a knee, an inner component can include fixed bone cuts, e.g., 5 fixed bone cuts on a femoral component. An outer component can be attachable to an inner component, wherein the outer component can have a patient-specific, patient-adapted shape and/or standard shape. The outer component can have an optional locking mechanism that combines the outer component with the inner component, with the integration or connecting feature of a standard size and/or shape, for any joint, including in a femur or tibia.

In one exemplary embodiment, the following modeling and derivation steps can be utilized to create a desired implant design:

(1) construct outer cartilage surface from edges of multiple faceted cuts;

(2) define multiple virtual bone cuts, extract sagittal curve, apply best fit analysis for closest implant, adapt best fit on AP length, ML etc, notch, condyle height;

(3) apply predefined virtual bone cuts according to design rules (AP height, best fit), if any;

(4) select implant; and

(5) optionally reduce number of cuts after surface has been constructed to obtain 5 cut inner surface system.

The same modeling and derivation processes can be applied to other joints, e.g., the hip or shoulder, where a series of cuts can be used to estimate or derive a shape or curvature, and wherein the shape or curvature information can be improved by combining it with information about other anatomic features and/or design, availability, cost or other constraints for the implant.

The selection of fit optimized bone cuts can alternatively help in achieving a good or better fit of the implant to the native, uncut articular surface.

### Generating an Articular Repair Suystem

In various embodiments, the articular repair systems (e.g., resection cut strategy, guide tools, and implant components) described herein can be formed or selected to achieve various parameters including a near anatomic fit or match with the surrounding or adjacent cartilage, subchondral bone, menisci and/or other tissue. If the articular repair system is intended to replace an area of diseased cartilage or lost cartilage, the near anatomic fit can be achieved using various methods that provides a virtual reconstruction of the shape of healthy cartilage in an electronic image.

In one embodiment, a near normal cartilage surface at the position of the cartilage defect can be reconstructed by interpolating the healthy cartilage surface across the cartilage defect or area of diseased cartilage. This can, for example, be achieved by describing the healthy cartilage by means of a parametric surface (e.g., a B-spline surface), for which the control points are placed such that the parametric surface follows the contour of the healthy cartilage and bridges the cartilage defect or area of diseased cartilage. The continuity properties of the parametric surface will provide a smooth integration of the part that bridges the cartilage defect or area of diseased cartilage with the contour of the surrounding healthy cartilage. The part of the parametric surface over the area of the cartilage defect or area of diseased cartilage can be used to determine the shape or part of the shape of the articular repair system to match with the surrounding cartilage.

In another embodiment, a near normal cartilage surface at the position of the cartilage defect or area of diseased cartilage can be reconstructed using morphological image processing. In a first step, the cartilage can be extracted from the electronic image using manual, semi-automated and/or automated segmentation techniques (e.g., manual tracing, region growing, live wire, model-based segmentation), resulting in a binary image. Defects in the cartilage appear as indentations that can be filled with a morphological closing operation performed in 2-D or 3-D with an appropriately selected structuring element. The closing operation is typically defined as a dilation followed by an erosion. A dilation operator sets the current pixel in the output image to 1 if at least one pixel of the structuring element lies inside a region in the source image. An erosion operator sets the current pixel in the output image to 1 if the whole structuring element lies inside a region in the source image. The filling of the cartilage defect or area of diseased cartilage creates a new surface over the area of the cartilage defect or area of diseased cartilage that can be used to determine the shape or part of the shape of the articular repair system to match with the surrounding cartilage or subchondral bone.

As described above, the articular repair system can be formed or selected from a library or database of systems of various sizes, including various medio-lateral (ML) antero-posterior (AP) and supero-inferior (SI) dimensions, curvatures and thicknesses, so that it achieves a near anatomic fit or match with the surrounding or adjacent cartilage, cortical bone, trabecular bone, subchondral bone, as well as cut bone, before or after preparing an implantation site. These systems can be pre-made or made to order for an individual patient. In order to control the fit or match of the articular repair system with the surrounding or adjacent cartilage, cortical bone, trabecular bone, subchondral bone, as well as cut bone before or after preparing an implantation site or menisci and other tissues preoperatively, a software program can be used that projects the articular repair system over the anatomic position where it will be implanted.

In yet another embodiment, the articular surface repair system can be projected over the implantation site prior to, during or after planning or simulating the surgery virtually using one or more 3-D images. The cartilage, cortical bone, trabecular bone, subchondral bone, as well as cut bone, before or after preparing an implantation site and other anatomic structures are extracted from a 3-D electronic image such as an MRI or a CT using manual, semi-automated and/or automated segmentation techniques. In select embodiments, segmentation may not be absolutely necessary and data can be directly utilized and/or displayed using grayscale image information.

Optionally, a 3-D representation of the cartilage, cortical bone, trabecular bone, subchondral bone, as well as cut bone, before or after preparing an implantation site and other anatomic structures as well as the articular repair system is generated, for example using a polygon or non-uniform rational B-spline (NURBS) surface or other parametric surface representation. For a description of various parametric surface representations see, for example Foley, J. D. et al., Computer Graphics: Principles and Practice in C; Addison-Wesley, 2nd edition (1995).

In various embodiments, an implant can be selected or designed so that not only one, but multiple patient specific resection surfaces will achieve a desired percentage coverage relative to the cut surface, e.g., 80%, 85%, 90%, 95%, 98%, 99%, 100%. This may be applicable to any joint that requires alteration of the articular surface for placement of an implant using, for example, cutting, milling, drilling, reaming etc. including a hip, a knee, an ankle, a foot, a toe, a shoulder, an elbow, a wrist, a hand, a finger, a spinal joint including an intervertebral disk space or a vertebral body.

The 3D representations of the cartilage, cortical bone, trabecular bone, subchondral bone, as well as cut bone, before or after preparing an implantation site and other anatomic structures and the articular repair system can be merged into a common coordinate system. The articular repair system can then be placed at the desired implantation site. The representations of the cartilage, cortical bone, trabecular bone, subchondral bone, as well as cut bone, before or after preparing an implantation site, menisci and other anatomic structures and the articular repair system are rendered into a 3-D image, for example application programming interfaces (APIs) OpenGL® (standard library of advanced 3-D graphics functions developed by SG), Inc.; available as part of the drivers for PC-based video cards, for example from www.nvidia.com for NVIDIA video cards or www.3dlabs.com for 3Dlabs products, or as part of the system software for Unix workstations) or DirectX® (multimedia API for Microsoft Windows® based PC systems; available from www.microsoft.com). The 3-D image can be rendered showing the cartilage, cortical bone, trabecular bone, subchondral bone, as well as cut bone, before or after preparing an implantation site, menisci or other anatomic objects, and the articular repair system from varying angles, e.g., by rotating or moving them interactively or non-interactively, in real-time or non-real-time.

The software can be designed so that the articular repair system, including surgical tools and instruments with the best fit relative to the cartilage, cortical bone, trabecular bone, subchondral bone, as well as cut bone, before or after preparing an implantation site is automatically selected, for example using some of the techniques described above. Alternatively, the operator of an semi-automated system can select an articular repair system, including surgical tools and instruments and project it or drag it onto the implantation site using suitable tools and techniques. The operator can move and rotate the articular repair system in three dimensions relative to the implantation site, cut or uncut, and can perform a visual inspection of the fit between the articular repair system and the implantation site, cut or uncut. The visual inspection can be computer assisted. The procedure can be repeated until a satisfactory fit has been achieved. The procedure can be performed manually by the operator; or it can be computer assisted in whole or part. For example, the software can select a first trial implant that the operator can test. The operator can evaluate the fit. The software can be designed and used to highlight areas of poor alignment between the implant and the surrounding cartilage or subchondral bone or menisci or other tissues. Based on this information, the software or the operator can then select another implant and test its alignment. One of skill in the art will readily be able to select, modify and/or create suitable computer programs for the purposes described herein.

In another embodiment, the implantation site can be visualized using one or more cross-sectional 2D images. Typically, a series of 2D cross-sectional images will be used. The 2D images can be generated with imaging tests such as CT, MRI, digital tomosynthesis, ultrasound, or optical coherence tomography using methods and tools known to those of skill in the art. The articular repair system can then be superimposed onto one or more of these 2-D images. The 2-D cross-sectional images can be reconstructed in other planes, e.g., from sagittal to coronal, etc. Isotropic data sets (e.g., data sets where the slice thickness is the same or nearly the same as the in-plane resolution) or near isotropic data sets can also be used. Multiple planes can be displayed simultaneously, for example using a split screen display. The operator can also scroll through the 2D images in any desired orientation in real-time or near-real-time; the operator can rotate the imaged tissue volume while doing this. The articular repair system can be displayed in cross-section utilizing different display planes, e.g., sagittal, coronal or axial, typically matching those of the 2-D images demonstrating the cartilage, cortical bone, trabecular bone, subchondral bone, as well as cut bone, before or after preparing an implantation site, menisci or other tissue. Alternatively or in addition, a three-dimensional display can be used for the articular repair system. The 2D electronic image and the 2D or 3-D representation of the articular repair system can be merged into a common coordinate system. The articular repair system can then be placed at the desired implantation site. The series of 2D cross-sections of the anatomic structures, the implantation site and the articular repair system can be displayed interactively (e.g., the operator can scroll through a series of slices) or noninteractively (e.g., as an animation that moves through the series of slices), in real-time or non-real-time.

In another embodiment, the fit between the implant and the implantation site can be evaluated. The implant can be available in a range of different dimensions, sizes, shapes and thicknesses. Different dimensions, sizes, shapes and thicknesses can be available for a medial condyle, a lateral condyle, a trochlea, a medial tibia, a lateral tibia, the entire tibia, a medial patella, a lateral patella, an entire patella, a medial trochlea, a central trochlea, a lateral trochlea, a portion of a femoral head, an entire femoral head, a portion of an acetabulum, an entire acetabulum, a portion of a glenoid, an entire glenoid, a portion of a humeral head, an entire humeral head, a portion of an ankle joint, an entire ankle joint, and/or a portion or an entire elbow, wrist, hand, finger, spine, or facet joint.

In certain embodiments, a combination of parameters can be selected. For example, one or more of an M-L measurement, an A-P measurement, and an S-I measurement of a patient's joint can be obtained from the subject preoperatively, for example, from one or more images of the subject's joint. Then, based on the one or measurements, an implant or implant component and associated surgical plan (and guide tools) for the subject's joint can be designed or selected preoperatively.

### Exemplary Implant Design #1

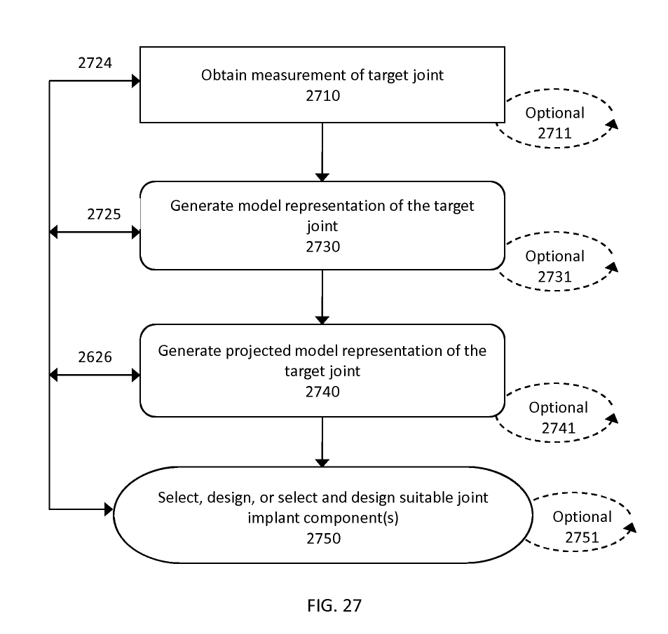

FIG. 27 is an illustrative flow chart showing exemplary steps taken by an automated program in assessing a joint and selecting and/or designing a suitable replacement implant component. First, the program obtains measurements of a target joint 2710. The step of obtaining a measurement can be accomplished, for example, based on an image of the joint. This step can be repeated 2711 as necessary to obtain a plurality of measurements, for example, from one or more images of the patient's joint, in order to further refine the joint assessment process. Once the program has obtained the necessary measurements, the information can be used to generate a model representation of the target joint being assessed 2730. This model representation can be in the form of a topographical map or image. The model representation of the joint can be in one, two, or three dimensions. It can include a virtual model and/or a physical model. More than one model can be created 2731, if desired. Either the original model, or a subsequently created model, or both can be used.

After the model representation of the joint is generated 2730, the program can generate a projected model representation of the target joint in a corrected condition 2740, e.g., based on a previous image of the patient's joint when it was healthy, based on an image of the patient's contralateral healthy joint, based on a projected image of a surface that negatively-matches the opposing surface, or a combination thereof. This step can be repeated 2741, as necessary or as desired. Using the difference between the topographical condition of the joint and the projected image of the joint, the program can then select a joint implant 2750 that is suitable to achieve the corrected joint anatomy. As will be appreciated by those of skill in the art, the selection and/or design process 2750 can be repeated 2751 as often as desired to achieve the desired result. Additionally, it is contemplated that the program can obtain a measurement of a target joint 2710 by obtaining, for example, an x-ray, and then selects a suitable joint replacement implant 2750.

One or more of these steps can be repeated reiteratively 2724, 2725, 2726. Moreover, the program can proceed directly from the step of generating a model representation of the target joint 2730 to the step of selecting a suitable joint implant component 2750. Additionally, following selection and/or design of the suitable joint implant component 2750, the steps of obtaining measurement of a target joint 2710, generating model representation of target joint 2730 and generating projected model 2740, can be repeated in series or parallel as shown by the flow 2724, 2725, 2726.

### Exemplary Implant Design #2

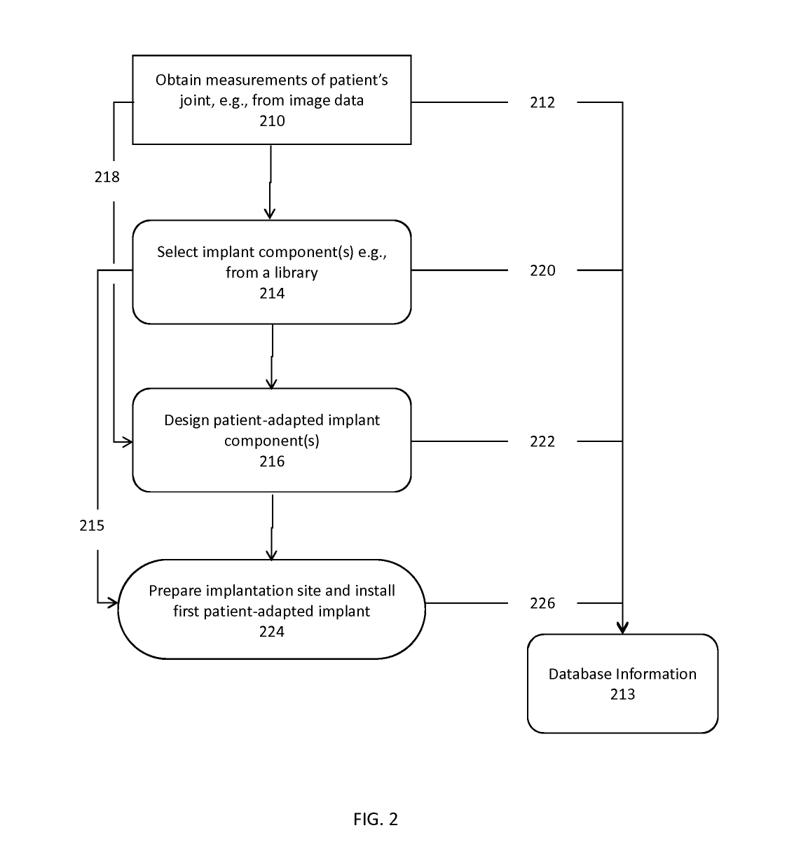

FIG. 2 is a flow chart illustrating a process for an automated system that includes selecting and/or designing a patient-adapted implant. First, using the techniques described herein or those suitable and known in the art, measurements of the target joint are obtained 210. This step can be repeated multiple times, as desired. Optionally, a virtual model of the joint can be generated, for example, to determine proper joint alignment and the corresponding resection cuts and implant component features based on the determined proper alignment. This information can be collected and stored 212 in a database 213. Once measurements of the target joint are obtained and analyzed to determine resection cuts and patient-adapted implant features, the patient-adapted implant components can be selected 214 (e.g., selected from a virtual library and optionally manufactured without further design alteration 215, or selected from a physical library of implant components). Alternatively, or in addition, one or more implant components with best-fitting and/or optimized features can be selected 214 (e.g., from a library) and then further designed (e.g., designed and manufactured) 216. Alternatively or in addition, one or more implant components with best-fitting and/or optimized features can be designed (e.g., designed and manufactured) 218, 216 without an initial selection from a library. Using a virtual model to assess the selected or designed implant component(s), this process also can be repeated as desired (e.g., before one or more physical components are selected and/or generated). The information regarding the selected and/or designed implant component(s) can be collected and stored 220, 222 in a database 213. Once a desired first patient-adapted implant component or set of implant components is obtained, a computer-controlled robotic surgical system or surgeon can prepare the implantation site and install the implant 224. The information regarding preparation of the implantation site and implant installation can be collected and stored 226 in a database 213. In this way, the information associated with the implant component is available for use in a subsequent surgery and/or during assessment of the current surgical repair (which may include a surgical assessment for subsequent implantation of a second or revision implant or component(s) thereof).

It should be understood that, as part of the previously described process, one or more databases of relevant patient data could be used by the automated system at virtually any point in the process to assess, compare, evaluate and/or modify specific relevant information. For example, the system could use a database of relevant non-patient anatomical data to assess, compare, evaluate and/or modify patient-specific information. Similarly, the system could use a database of relevant non-patient anatomical models to assess, compare, evaluate and/or modify patient-specific models. Similar databases could be used to assess, compare, evaluate and/or modify patient-specific surgical bone cuts, procedures, implant features, manufacturing methods and/or robotic surgical steps. By providing such relevant databases, the computer can potentially identify and utilize similar solutions from other surgical and/or design plans without requiring intervention by a human operator. Moreover, where the system is unable to identify relevant or similar data (or confidence in such computer solutions is low), the system can flag or otherwise identify the issue which can potentially notify a human operator that such intervention may be necessary and/or desired.

### Exemplary Implant Design #3

In another exemplary embodiment, the steps described herein can be performed in any order and can be performed more than once in a particular process. For example, one or more steps can be reiterated and refined a second, third, or more times, before, during, or after performing other steps or sets of steps in the process. While this process specifically describes steps for selecting and/or designing a patient-specific total knee implant, it can be adapted to design other embodiments, for example, patient-adapted bicompartmental knee implants, unicompartmental knee implants, and implants for shoulders and hips, vertebrae, and other joints.

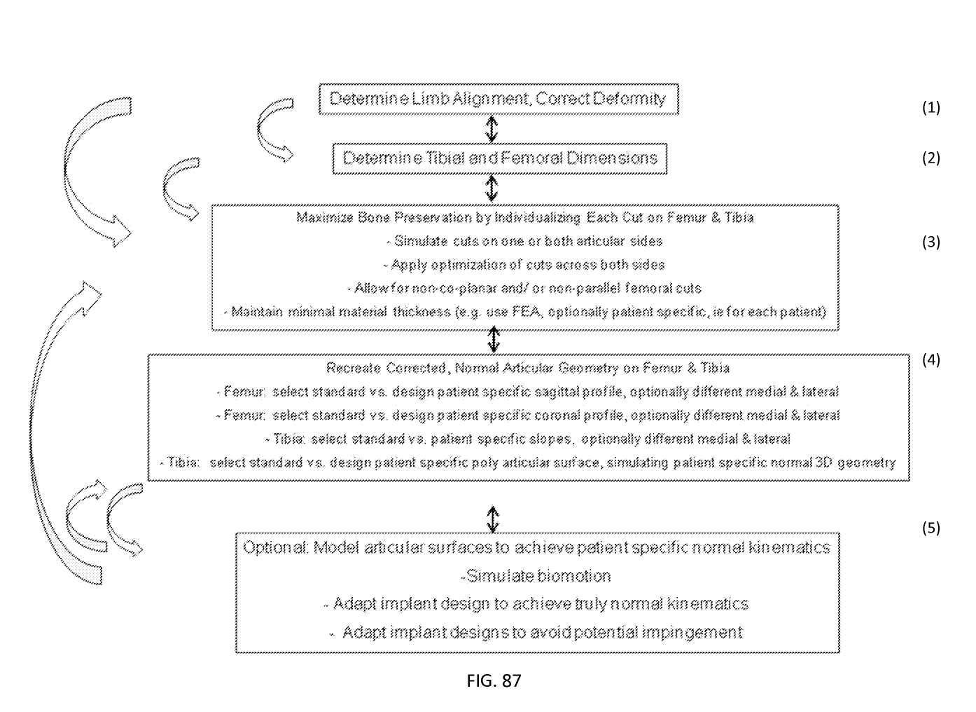

The exemplary process shown in FIG. 87 includes four general steps and, optionally, can include a fifth general step. Each general step includes various specific steps. The general steps are identified as (1)-(5) in the figure. These steps can be performed virtually, for example, by using one or more computers that have or can receive patient-specific data and specifically configured software or instructions to perform such steps.

In general step (1), limb alignment and deformity corrections are determined, to the extent that either is needed for a specific patient's situation.

In general step (2), the requisite tibial and femoral dimensions of the implant components are determined based on patient-specific data obtained, for example, from image data of the patient's knee.

In general step (3), bone preservation is maximized by virtually determining a resection cut strategy for the patient's femur and/or tibia that provides minimal bone loss optionally while also meeting other user-defined parameters such as, for example, maintaining a minimum implant thickness, using certain resection cuts to help correct the patient's misalignment, removing diseased or undesired portions of the patient's bone or anatomy, and/or other parameters. This general step can include one or more of the steps of (i) simulating resection cuts on one or both articular sides (e.g., on the femur and/or tibia), (ii) applying optimized cuts across one or both articular sides, (iii) allowing for non-co-planar and/or non-parallel femoral resection cuts (e.g., on medial and lateral corresponding portions of the femur) and, optionally, non-co-planar and/or non-parallel tibial resection cuts (e.g., on medial and lateral corresponding portions of the tibia), and (iv) maintaining and/or determining minimal material thickness. The minimal material thickness for the implant selection and/or design can be an established threshold, for example, as previously determined by a finite element analysis (“FEA”) of the implant's standard characteristics and features. Alternatively, the minimal material thickness can be determined for the specific implant, for example, as determined by an FEA of the implant's standard and patient-specific characteristics and features. If desired, FEA and/or other load-bearing/modeling analysis may be used to further optimize or otherwise modify the individual implant design, such as where the implant is under or over-engineered than required to accommodate the patient's biomechanical needs, or is otherwise undesirable in one or more aspects relative to such analysis. In such a case, the implant design may be further modified and/or redesigned to more accurately accommodate the patient's needs, which may have the side effect of increasing/reducing implant characteristics (i.e., size, shape or thickness) or otherwise modifying one or more of the various design “constraints” or limitations currently accommodated by the present design features of the implant. If desired, this step can also assist in identifying for a surgeon the bone resection design to perform in the surgical theater and it also identifies the design of the bone-facing surface(s) of the implant components, which substantially negatively-match the patient's resected bone surfaces, at least in part.

In general step (4), a corrected, normal and/or optimized articular geometry on the femur and tibia is recreated virtually. For the femur, this general step can include, for example, the step of: (i) selecting a standard sagittal profile, or selecting and/or designing a patient-engineered or patient-specific sagittal profile; and (ii) selecting a standard coronal profile, or selecting and/or designing a patient-specific or patient-engineered coronal profile. Optionally, the sagittal and/or coronal profiles of one or more corresponding medial and lateral portions (e.g., medial and lateral condyles) can include different curvatures. For the tibia, this general step includes one or both of the steps of: (iii) selecting a standard anterior-posterior slope, and/or selecting and/or designing a patient-specific or patient-engineered anterior-posterior slope, either of which optionally can vary from medial to lateral sides; and (iv) selecting a standard poly-articular surface, or selecting and/or designing a patient-specific or patient-engineered poly-articular surface. The patient-specific poly-articular surface can be selected and/or designed, for example, to simulate the normal or optimized three-dimensional geometry of the patient's tibial articular surface. The patient-engineered poly-articular surface can be selected and/or designed, for example, to optimize kinematics with the bearing surfaces of the femoral implant component. This step can be used to define the bearing portion of the outer, joint-facing surfaces (i.e., articular surfaces) of the implant components.

In optional general step (5), a virtual implant model (for example, generated and displayed using a computer specifically configured with software and/or instructions to assess and display such models) is assessed and can be altered to achieve normal or optimized kinematics for the patient. For example, the outer joint-facing or articular surface(s) of one or more implant components can be assessed and adapted to improve kinematics for the patient. This general step can include one or more of the steps of: (i) virtually simulating biomotion of the model, (ii) adapting the implant design to achieve normal or optimized kinematics for the patient, and (iii) adapting the implant design to avoid potential impingement.

The exemplary process described above facilitates the automated creation of both a predetermined surgical resection design for altering articular surfaces of a patient's bones during surgery and a design for an implant that specifically fits the patient, for example, following the surgical bone resectioning. Specifically, the implant selection and/or design, which can include manufacturing or machining the implant to the selected and/or designed specifications using known techniques, includes one or more patient-engineered bone-facing surfaces that negatively-match the patient's resected bone surface. The implant also can include other features that are patient-adapted, such as minimal implant thickness, articular geometry, and kinematic design features. This process can be applied to various joint implants and to various types of joint implants. For example, this design process can be applied to a total knee, cruciate retaining, posterior stabilized, and/or ACL/PCL retaining knee implants, bicompartmental knee implants, unicompartmental knee implants, and other joint implants, for example, for the shoulder, hip, elbow, spine, or other joints. For example, the thickness of an acetabular cup, either metal backing or polyethylene or ceramic or other insert, can be adapted based on the patient's geometry, e.g., depth of the actebular fossa, AP, ML, SI dimensions or other parameters including femoral parameters.

Another advantage to this process is that the selection and/or design process can incorporate any number of target parameters such that any number of implant component features and resection cuts can be selected and/or designed to meet one or more parameters that are predetermined to have clinical value. For example, in addition to bone preservation, a selection and/or design process can include target parameters to restore a patient's native, normal kinematics, or to provide optimized kinematics. For example, the process for selecting and/or designing an implant and/or resection cuts can include target parameters such as reducing or eliminating the patient's mid-flexion instability, reducing or eliminating “tight” closure, improving or extending flexion, improving or restoring cosmetic appearance, and/or creating or improving normal or expected sensations in the patient's knee. The design for a tibial implant can provide an engineered surface that replicates the patient's normal anatomy yet also allows for low contact stress on the tibia.

This process can also provide a simplified surgical technique. The selected and/or designed bone cuts and, optionally, other features that provide a patient-adapted fit for the implant components eliminates the complications that arise in the surgical setting with traditional, misfitting implants. Moreover, since the process and implant component features are predetermined prior to surgery, model images of the surgical steps can be provided to the surgeon as a guide.

In various embodiments, the design of an implant component can include manufacturing or machining the component in accordance with the implant design specifications. Manufacturing can include, for example, using a designed mold to form the implant component. Machining can include, for example, altering a selected blank form to conform to the implant design specifications.

### Libraries

As described herein, implants of various sizes, shapes, curvatures and thicknesses with various types and locations and orientations and number of bone cuts can be selected and/or designed and manufactured. The implant designs and/or implant components can be selected from, catalogued in, and/or stored in a library. The library can be a virtual library of implants, or components, or component features that can be combined and/or altered to create a final implant. The library can include a catalogue of physical implant components. In certain embodiments, physical implant components can be identified and selected using the library. The library can include previously-generated implant components having one or more patient-adapted features, and/or components with standard or blank features that can be altered to be patient-adapted. Accordingly, implants and/or implant features can be selected from the library. Libraries may also be created that contain associated surgical procedures and/or guide tool designs, in a similar manner.

A virtual or physical implant component can be selected from the library based on similarity to prior or baseline parameter optimizations, such as one or more of (1) deformity correction and limb alignment (2) maximum preservation of bone, cartilage, or ligaments, (3) preservation and/or optimization of other features of the patient's biology, such as trochlea and trochlear shape, (4) restoration and/or optimization of joint kinematics, and (5) restoration or optimization of joint-line location and/or joint gap width. Accordingly, one or more implant component features, such as (a) component shape, external and/or internal, (b) component size, and/or (c) component thickness, can be determined precisely and/or determined within a range from the library selection. Then, the selected implant component can be designed or engineered further to include one or more patient-specific features. For example, a joint can be assessed in a particular subject and a pre-existing implant design having the closest shape and size and performance characteristics can be selected from the library for further manipulation (e.g., shaping) and manufacturing prior to implantation. For a library including physical implant components, the selected physical component can be altered to include a patient-specific feature by adding material (e.g., laser sintering) and/or subtracting material (e.g., machining).

Accordingly, in certain embodiments an implant can include one or more features designed patient-specifically and one or more features selected from one or more library sources. For example, in designing an implant for a total knee replacement comprising a femoral component and a tibial component, one component can include one or more patient-specific features and the other component can be selected from a library. Table 6 includes an exemplary list of possible combinations.

TABLE 6
Illustrative Combinations of Patient-Specific and
Library-Derived Components

| | Implant component(s) | Implant component(s) |
|---|---|---|
| Implant | having a patient-specific | having a library derived |
| component(s) | feature | feature |
| Femoral, Tibial | Femoral and Tibial | Femoral and Tibial |
| Femoral, Tibial | Femoral | Femoral and Tibial |
| Femoral, Tibial | Tibial | Femoral and Tibial |
| Femoral, Tibial | Femoral and Tibial | Femoral |
| Femoral, Tibial | Femoral and Tibial | Tibial |
| Femoral, Tibial | Femoral and Tibial | none |

In certain embodiments, a library can be generated to include images from a particular patient at one or more ages prior to the time that the patient needs a joint implant. For example, a method can include identifying patients eliciting one or more risk factors for a joint problem, such as low bone mineral density score, and collecting one or more images of the patient's joints into a library. In certain embodiments, all patients below a certain age, for example, all patients below 40 years of age can be scanned to collect one or more images of the patient's joint. The images and data collected from the patient can be banked or stored in a patient-specific database. For example, the articular shape of the patient's joint or joints can be stored in an electronic database until the time when the patient needs an implant. Then, the images and data in the patient-specific database can be accessed and a patient-specific and/or patient-engineered partial or total joint replacement implant using the patient's original anatomy, not affected by arthritic deformity yet, can be generated. This process results in a more functional and more anatomic implant.

### Finalizing Implant and Evaluating Function/Anatomy

In various embodiments, once the desired features of the various implant components have been designed and/or selected (i.e., inner bone-facing surface features, outer joint facing surfaces, perimeter edge, anchoring features, thickness of implant in one or more regions, etc.), a complete model of the implant component can be synthesized. In various embodiments, the model and/or model data can be used for selection and/or design of resection cuts, guide tools, and/or implant components, which can be included in the same model or in a different model. For example, the model and/or model data can be exported to a CAD program, for example, SolidWorks, to design one or more patient-engineered resection cuts, patient-specific guide tools, and/or patient-adapted implant components. Alternatively or in addition, the model and/or model data can be exported to a library, for example, to be included in the library as a template model for future assessments and/or for selecting from the library one or more resection cut strategies, guide tools, and/or implant components.

In another embodiment, the virtual model includes, in addition to or instead of the surface model representation, a template for one or more implants and/or guide tools, including the position and shape of bearing surfaces as well as the location and direction of bone cuts and/or drill holes needed to position the implants. Similar to the way the surface data representation is adjusted using global transformations and local deformations as described above to match the individual patient's anatomy, the shape of the implants and/or guide tools can be adjusted accordingly, i.e. the software applies the same global transformations and local deformations applied to the surface model to the implants and/or guide tools as well. During this process, the position and shape of the bearing surfaces as well as the position and direction of bone cuts and/or drill holes can be adjusted as well based on the transformations and deformations of the virtual shape model. Adjusting the position and shape of bearing surfaces and the position and direction of bone cuts and/or drill holes can be performed automatically by the software or based on user or operator input or a combination thereof

In another embodiment, the global transformations and local deformations of various surface models as well as the implant, guide tool, bearing surface, bone cut and drill hole information are determined to not only match the surface of the patient's biological structure of interest, but also taking into account anatomical landmarks of the patient's individual anatomy. This can include, for example and without limitation, the femoral sulcus line, the femoral notch, the femoral trochlea, the cruciate ligaments, the medial and/or lateral tibial spine, the anterior or posterior femoral shaft cortex, the medial or lateral margin of the patella, the anterior or posterior margin of the medial or lateral tibial plateau or the margin of the femoral or tibial articular surface. The shape of the patient's anatomy, for example the shape of one or more articular surfaces, can also be used.

In another embodiment, the position and/or direction of the implant, guide tool and/or bone cuts and drill holes is determined, at least in part, based on axis information of the patient's individual anatomy, for example an anatomical or a mechanical axis of the patient's knee.

In another embodiment, the global transformations and local deformations are determined by the software, at least in part, based on external design constraints pertinent to a particular implant design. This can include, for example, specific surface curvature radii, minimum distance between structures such as anchoring elements and/or minimum or maximum thickness or length or width dimensions of the implant or parts thereof. The transformations can also be optimized to minimize bone cuts.

In a further embodiment, the model or template of the implants and/or guide tools can be fit to the patient's anatomy after the axis alignment of the joint, for example the anatomical or biomechanical axis, has been corrected. The fitting, optimization or deformation of the model or template can then be performed taking the corrected axis into account. Alternatively, the axis alignment is corrected after the model has been fitted. The model can then undergo further adjustments as the alignment correction is performed. Thus, the position or shape of the bearing surfaces and the position and/or direction of the implant, guide tool and/or bone cuts and drill holes is determined based on the corrected axis information.

In another embodiment, the virtual model includes, in addition to or instead of the surface model representation, one or more geometric reference structures. This can include, for example, planes, axes, curves or surfaces that are used as construction parameters for one or more implants and/or guide tools. The geometric reference structures can be used to define the position and shape of bearing surfaces as well as the location and direction of bone cuts and/or drill holes needed to position the implants. Similar to the way the surface data representation is adjusted using global transformations and local deformations as described above to match the individual patient's anatomy, the position, direction, scale and/or shape of the geometric reference structures can be adjusted accordingly, i.e. the software applies the same global transformations and local deformations applied to the surface model to the geometric reference structures as well. During this process, the position, direction, scale and/or shape of the geometric reference structures can be adjusted as well based on the transformations and deformations of the virtual shape model. Adjusting the position, direction, scale and shape of the geometric reference structures can be performed automatically by the software or based on user or operator input or a combination thereof

Once the adjustment of the geometric reference structures is complete, they can be used as construction parameters for the implants and/or guide tool. For example, reference planes can be used to define bone cuts of the implant and associated cut guides. Reference axes can serve to define the direction of anchoring pegs and the holes that need to be drilled. Reference curves can define the outer margin of an implant. Reference surfaces can define the bearing surface of an implant.

### Deformity Correction

As part of an implant final design, an automated or semi-automated system can consider information regarding the misalignment and the proper mechanical alignment of a patient's limb (i.e., from anatomical information and/or models thereof), which can be used to preoperatively design and/or select one or more features of a joint implant and/or implant procedure. For example, based on the difference between the patient's misalignment and the proper mechanical axis, a knee implant and implant procedure can be designed and/or selected preoperatively to include implant and/or resection dimensions that substantially realign the patient's limb to correct or improve a patient's alignment deformity. In addition, the process can include selecting and/or designing one or more surgical tools (e.g., guide tools or cutting jigs) to direct a clinician or robotic surgical system in resectioning the patient's bone in accordance with the preoperatively designed and/or selected resection dimensions.

In certain embodiments, the degree of deformity correction that is necessary to establish a desired limb alignment can be calculated based on information from the alignment of a virtual model of a patient's limb. The virtual model can be generated from patient-specific data, such 2D and/or 3D imaging data of the patient's limb. The deformity correction can correct varus or valgus alignment or antecurvatum or recurvatum alignment. In a preferred embodiment, the desired deformity correction returns the leg to normal alignment, for example, a zero degree biomechanical axis in the coronal plane and absence of genu antecurvatum and recurvatum in the sagittal plane.

### Attaining Acceptable Joint Kinematics

In certain embodiments, bone cuts and implant shape including at least one of a bone-facing or a joint-facing surface of the implant can be designed or selected to achieve normal joint kinematics. In certain embodiments, a computer program simulating biomotion of one or more joints, such as, for example, a knee joint, or a knee and ankle joint, or a hip, knee and/or ankle joint can be utilized. In certain embodiments, patient-specific imaging data can be fed into this computer program. For example, a series of two-dimensional images of a patient's knee joint or a three-dimensional representation of a patient's knee joint can be entered into the program. Additionally, two-dimensional images or a three-dimensional representation of the patient's ankle joint and/or hip joint may be added.

Alternatively, patient-specific kinematic data, for example obtained in a gait lab, can be fed into the computer program. Alternatively, patient-specific navigation data, for example generated using a surgical navigation system, image guided or non-image guided can be fed into the computer program. This kinematic or navigation data can, for example, be generated by applying optical or RF markers to the limb and by registering the markers and then measuring limb movements, for example, flexion, extension, abduction, adduction, rotation, and other limb movements.

Optionally, other data including anthropometric data may be added for each patient. These data can include but are not limited to the patient's age, gender, weight, height, size, body mass index, and race. Desired limb alignment and/or deformity correction can be added into the model. The position of bone cuts on one or more articular surfaces as well as the intended location of implant bearing surfaces on one or more articular surfaces can be entered into the model.

A patient-specific biomotion model can be derived that includes combinations of parameters listed above. The biomotion model can simulate various activities of daily life including normal gait, stair climbing, descending stairs, running, kneeling, squatting, sitting and any other physical activity. The biomotion model can start out with standardized activities, typically derived from reference databases. These reference databases can be, for example, generated using biomotion measurements using force plates and motion trackers using radiofrequency or optical markers and video equipment.

The biomotion model can then be individualized with use of patient-specific information including at least one of, but not limited to the patient's age, gender, weight, height, body mass index, and race, the desired limb alignment or deformity correction, and the patient's imaging data, for example, a series of two-dimensional images or a three-dimensional representation of the joint for which surgery is contemplated.

An implant shape including associated bone cuts generated in the preceding optimizations, for example, limb alignment, deformity correction, bone preservation on one or more articular surfaces, can be introduced into the model. Table 7 includes an exemplary list of parameters that can be measured in a patient-specific biomotion model.

TABLE 7
Parameters measured in a patient-specific biomotion model for various implants

| Joint implant | Measured Parameter |
|---|---|
| Knee | Medial femoral rollback during flexion |
| Knee | Lateral femoral rollback during flexion |
| Knee | Patellar position, medial, lateral, superior, inferior for different flexion |
| | and extension angles |
| Knee | Internal and external rotation of one or more femoral condyles |
| Knee | Internal and external rotation of the tibia |
| Knee | Flexion and extension angles of one or more articular surfaces |
| Knee | Anterior slide and posterior slide of at least one of the medial and |
| | lateral femoral condyles during flexion or extension |
| Knee | Medial and lateral laxity throughout the range of motion |
| Knee | Contact pressure or forces on at least one or more articular surfaces, |
| | e.g., a femoral condyle and a tibial plateau, a trochlea and a patella |
| Knee | Contact area on at least one or more articular surfaces, e.g., a femoral |
| | condyle and a tibial plateau, a trochlea and a patella |
| Knee | Forces between the bone-facing surface of the implant, an optional |
| | cement interface and the adjacent bone or bone marrow, measured at |
| | least one or multiple bone cut or bone-facing surface of the implant on |
| | at least one or multiple articular surfaces or implant components. |
| Knee | Ligament location, e.g., ACL, PCL, MCL, LCL, retinacula, joint |
| | capsule, estimated or derived, for example using an imaging test. |
| Knee | Ligament tension, strain, shear force, estimated failure forces, loads for |
| | example for different angles of flexion, extension, rotation, abduction, |
| | adduction, with the different positions or movements optionally |
| | simulated in a virtual environment. |
| Knee | Potential implant impingement on other articular structures, e.g., in |
| | high flexion, high extension, internal or external rotation, abduction or |
| | adduction or any combinations thereof or other angles/positions/ |
| | movements. |
| Hip, shoulder or | Internal and external rotation of one or more articular surfaces |
| other joint | |
| Hip, shoulder or | Flexion and extension angles of one or more articular surfaces |
| other joint | |
| Hip, shoulder or | Anterior slide and posterior slide of at least one or more articular |
| other joint | surfaces during flexion or extension, abduction or adduction, elevation, |
| | internal or external rotation |
| Hip, shoulder or | Joint laxity throughout the range of motion |
| other joint | |
| Hip, shoulder or | Contact pressure or forces on at least one or more articular surfaces, |
| other joint | e.g., an acetabulum and a femoral head, a glenoid and a humeral head |
| Hip, shoulder or | Forces between the bone-facing surface of the implant, an optional |
| other joint | cement interface and the adjacent bone or bone marrow, measured at |
| | least one or multiple bone cut or bone-facing surface of the implant on |
| | at least one or multiple articular surfaces or implant components. |
| Hip, shoulder or | Ligament location, e.g., transverse ligament, glenohumeral ligaments, |
| other joint | retinacula, joint capsule, estimated or derived, for example using an |
| | imaging test. |
| Hip, shoulder or | Ligament tension, strain, shear force, estimated failure forces, loads |
| other joint | for example for different angles of flexion, extension, rotation, |
| | abduction, adduction, with the different positions or movements |
| | optionally simulated in a virtual environment. |
| Hip, shoulder or | Potential implant impingement on other articular structures, e.g., in |
| other joint | high flexion, high extension, internal or external rotation, abduction or |
| | adduction or elevation or any combinations thereof or other angles/ |
| | positions/movements. |

The above list is not meant to be exhaustive, but only exemplary. Any other biomechanical parameter known in the art can be included in the analysis.

The resultant biomotion data can be used to further optimize the implant design with the objective to establish normal or near normal kinematics. The implant optimizations can include one or multiple implant components. Implant optimizations based on patient-specific data including image based biomotion data include, but are not limited to:
Changes to external, joint-facing implant shape in coronal plane
Changes to external, joint-facing implant shape in sagittal plane
Changes to external, joint-facing implant shape in axial plane
Changes to external, joint-facing implant shape in multiple planes or three dimensions
Changes to internal, bone-facing implant shape in coronal plane
Changes to internal, bone-facing implant shape in sagittal plane
Changes to internal, bone-facing implant shape in axial plane
Changes to internal, bone-facing implant shape in multiple planes or three dimensions
Changes to one or more bone cuts, for example with regard to depth of cut, orientation of cut

Any single one or combinations of the above or all of the above on at least one articular surface or implant component or multiple articular surfaces or implant components.

When changes are made on multiple articular surfaces or implant components, these can be made in reference to or linked to each other. For example, in the knee, a change made to a femoral bone cut based on patient-specific biomotion data can be referenced to or linked with a concomitant change to a bone cut on an opposing tibial surface, for example, if less femoral bone is resected, the computer program may elect to resect more tibial bone.

Similarly, if a femoral implant shape is changed, for example on an external surface, this can be accompanied by a change in the tibial component shape. This is, for example, particularly applicable when at least portions of the tibial bearing surface negatively-match the femoral joint-facing surface. These linked changes also can apply for hip and/or shoulder implants. Any combination is possible for virtually any joint as it pertains to the shape, orientation, and size of implant components on two or more opposing surfaces.

By optimizing implant shape in this manner, it is possible to establish normal or near normal kinematics. Moreover, it is possible to avoid implant related complications, including but not limited to anterior notching, notch impingement, posterior femoral component impingement in high flexion, and other complications associated with existing implant designs. For example, certain designs of the femoral components of traditional knee implants have attempted to address limitations associated with traditional knee implants in high flexion by altering the thickness of the distal and/or posterior condyles of the femoral implant component or by altering the height of the posterior condyles of the femoral implant component. Since such traditional implants follow a one-size-fits-all approach, they are limited to altering only one or two aspects of an implant design. However, with the design approaches described herein, various features of an implant component can be designed for an individual to address multiple issues, including issues associated with high flexion motion. For example, designs as described herein can alter an implant component's bone-facing surface (for example, number, angle, and orientation of bone cuts), joint-facing surface (for example, surface contour and curvatures) and other features (for example, implant height, width, and other features) to address issues with high flexion together with other issues.

Biomotion models for a particular patient can be supplemented with patient-specific finite element modeling or other biomechanical models known in the art. Resultant forces in the knee joint can be calculated for each component for each specific patient. The implant can be engineered to the patient's load and force demands. For instance, a 125 lb. patient may not need a tibial plateau as thick as a patient with 280 lbs. Similarly, the polyethylene can be adjusted in shape, thickness and material properties for each patient. For example, a 3 mm polyethylene insert can be used in a light patient with low force and a heavier or more active patient may need an 8 mm polymer insert or similar device.

### Joint-Line Location and Joint-Gap Width

Various embodiments include the use of anatomical and/or modeling data to assist an automated and/or semi-automated program in designing and/or selecting implant components, and related designs and methods, having one or more features that are engineered from patient-specific data to restore or optimize the particular patient's joint-line location. In addition or alternatively, certain patient-specific implant components, and related designs and methods, can have one or more features that are engineered from patient-specific data to restore or optimize the particular patient's joint gap width.

By positively-matching the implant component thickness profile with the cut depth profile, and by negatively-matching the component bone-facing surface with the resected articular surface of the biological structure, certain features of the component joint-facing surface can positively-match the corresponding biological features that it replaces. For example, if the component bone-facing surface and thickness match the corresponding features of the biological structure, the component joint-facing curvature, such as a j-curve, also can match the corresponding surface curvature of the patient's biological structure.

If desired, the one or more materials and/or material properties of an implant can be varied to accommodate unique or localized requirements. For example, it may be desirable for the strength and/or elasticity of the polymer in a tibial tray insert to vary along the surface or cross-sectional profile of the implant. In a similar manner, it may be desirous for a surface of such an implant to posses differing mechanical properties than subsurface portions of the implant. Likewise, it may be desirous for a periphery of such an implant to posses differing mechanical properties than central portions of the implant. In such a case, it may be advantageous to alter the material properties of such an implant in some manner, such as by chemical or physical processing or crosslinking (via chemical vulcanization or via low or high-energy irradiation), to accommodate the varying demands placed upon the polymer implant. Alternatively, the implant may comprise various materials that are adhered, layered or otherwise arranged in some fashion to accomplish various objectives of the present invention. In a similar manner, implants comprising metals and/or ceramic constituents may be formed of two or more materials, or may comprise a single material with sections or portions having varying material characteristics (i.e., by radiation, heating, cooling, hipping, annealing, chemical action, work hardening, peening, carburizing, hardening, surface treating, oxidation, etc.) For example, the medial and/or lateral and/or superior and/or inferior portions of a tibial tray inset maybe formed from two or more materials adhered or otherwise connected in some manner, each material having a unique material property, resulting in an implant with differing mechanical properties on its medial and/or lateral and/or superior and/or inferior sides. Such an implant could alternatively comprise a multi-layered material, with different materials exposed on the surface during the machining process (with the processing tools extending to differing depths), thereby resulting in a generally uniform layered material with different surface properties on the surface of its medial and lateral sides.

In certain embodiments, one or more implant components can designed based on patient-specific data to include a thickness profile that retains or alters a particular patient's joint gap width to retain or correct another patient-specific feature. For example, the patient-specific data can include data regarding the length of the patient's corresponding limbs (e.g., left and right limbs) and the implant component(s) can be designed to, at least in part, alter the length of one limb to better match the length of the corresponding limb.

If desired, the automated and/or semi-automated program can make adjustments of implant position and/or orientation such as rotation, bone cuts, cut height and selected component thickness, insert thickness or selected component shape or insert shape. In this manner, an optimal compromise can be found, for example, between biomechanical alignment and joint laxity or biomechanical alignment and joint function, e.g., in a knee joint flexion gap and extension gap. Thus, multiple approaches exist for optimizing soft-tissue tension, ligament tension, ligament balance, and/or flexion and extension gap. These include, for example, one or more of the exemplary options described in Table 8.

TABLE 8
Exemplary approach options for optimizing soft-tissue tension, ligament
tension, ligament balance, and/or flexion and extension gap

| Option # | Description of Exemplary Option |
|---|---|
| 1 | Position of one or more femoral bone cuts |
| 2 | Orientation of one or more femoral bone cuts |
| 3 | Location of femoral component |
| 4 | Orientation of femoral component, including rotational |
| | alignment in axial, sagittal and coronal direction |
| 5 | Position of one or more tibial bone cuts |
| 6 | Orientation of one or more tibial bone cuts including sagittal |
| | slope, mediolateral orientation |
| 7 | Location of tibial component |
| 8 | Orientation of tibial component, including rotational |
| | alignment in axial, sagittal and coronal direction |
| 9 | Tibial component height |
| 10 | Medial tibial insert or component or composite height |
| 11 | Lateral tibial insert or component or composite height |
| 12 | Tibial component profile, e.g., convexity, concavity, trough, |
| | radii of curvature |
| 13 | Medial tibial insert or component or composite profile, e.g., |
| | convexity, concavity, trough, radii of curvature |
| 14 | Lateral tibial insert or component or composite profile, e.g., |
| | convexity, concavity, trough, radii of curvature |
| 15 | Select soft-tissue releases, e.g., partial or full releases of |
| | retinacula and/or ligaments, “pie-crusting” etc. |

Any one option described in Table 8 can be optimized alone or in combination with one or more other options identified in the table and/or known in the art for achieving different flexion and extension, abduction, or adduction, internal and external positions and different kinematic requirements.

In one embodiment, robotic surgical equipment can initially optimize the femoral and tibial resections. Optimization can be performed by measuring soft-tissue tension or ligament tension or balance for different flexion and extension angles or other joint positions before any bone has been resected, once a first bone resection on a first articular surface has been made and after a second bone resection on a first or second articular surface has been made, such as a femur and a tibia, humerus and a glenoid, femur and an acetabulum.

The position and orientation between a first implant component and a second, opposing implant component or a first articular surface and a trial implant or a first trial implant and a second trial implant or an alignment guide and an instrument guide and any combinations thereof can be optimized with the use of, for example, interposed spacers, wedges, screws and other mechanical or electrical methods known in the art. The robotic surgical equipment may desire to influence joint laxity as well as joint alignment. This can be optimized for different flexion and extension, abduction, or adduction, internal and external rotation angles. For this purpose, spacers can be introduced at or between one or more steps in the implant procedure. One or more of the spacers can be attached or in contact with one or more instruments, trials or, optionally, patient-specific molds. The robotic surgical equipment can intraoperatively evaluate the laxity or tightness of a joint using spacers with different thicknesses or one or more spacers with the same thickness. For example, spacers can be applied in a knee joint in the presence of one or more trials or instruments or patient-specific molds and the flexion gap can be evaluated with the knee joint in flexion. The knee joint can then be extended and the extension gap can be evaluated. Ultimately, the robotic surgical equipment selects for a given joint an optimal combination of spacers and trial or instrument or patient-specific mold. A surgical cut guide can be applied to the trial or instrument or patient-specific mold with the spacers optionally interposed between the trial or instrument or patient-specific mold and the cut guide. In this manner, the exact position of the surgical cuts can be influenced and can be adjusted to achieve an optimal result. Someone skilled in the art will recognize other means for optimizing the position of the surgical cuts. For example, expandable or ratchet-like devices can be utilized that can be inserted into the joint or that can be attached or that can touch the trial or instrument or patient-specific mold. Hinge-like mechanisms are applicable. Similarly, jack-like mechanisms are useful. In principal, any mechanical or electrical device useful for fine tuning the position of a cut guide relative to a trial or instrument or patient—specific mold can be used.

The robotic surgical equipment may choose to influence joint laxity as well as joint alignment. This can be optimized for different flexion and extension, abduction, or adduction, internal and external rotation angles. For this purpose, for example, spacers can be introduced that are attached or that are in contact with one or more trials or instruments or patient-specific molds. The robotic surgical equipment can intraoperatively evaluate the laxity or tightness of a joint using spacers with different thickness or one or more spacers with the same thickness. For example, spacers can be applied in a knee joint in the presence of one or more instruments or trials or molds and the flexion gap can be evaluated with the knee joint in flexion. Different thickness trials can be used. The terms spacer or insert can be used interchangeably with the term trial.

In certain embodiments, the robotic surgical equipment can introduce different trials or spacers or instruments of different thicknesses in the medial and/or lateral joint space in a knee. This can be done before any bone has been resected, once a first bone resection on a first articular surface has been made and after a second bone resection on a first or second articular surface has been made, such as a femur and a tibia or a medial and a lateral condyle or a medial and a lateral tibia. The joint can be tested for soft-tissue tension, ligament tension, ligament balance and/or flexion or extension gap for different orientations or kinematic requirements using different medial and lateral trial or spacer thicknesses, e.g., at different flexion and extension angles. Surgical bone cuts can subsequently optionally be adapted or changed. Alternatively, different medial and lateral insert thickness or profiles or composite heights can be selected for the tibial component(s).

Thus, by using separate medial and/or lateral spacers or trials or inserts, it is possible to determine an optimized combination of medial or lateral tibial components, for example with regard to medial and lateral composite thickness, insert thickness or medial and lateral implant or insert profile. Thus, medial and/or lateral tibial implant or component or insert thickness can be optimized for a desired soft-tissue or ligament tension or ligament balance for different flexion and extension angles and other joint poses. This offers a unique benefit beyond traditional balancing using bone cuts and soft-tissue releases. In one embodiment, the robotic surgical equipment can place the tibial and femoral surgical bone cuts and perform the proper soft-tissue or ligament tensioning or balancing entirely via selection of a medial or lateral tibial insert or composite thickness and/or profile. Additional adaptation and optimization of bone cuts and soft-tissue releases is possible.

The following example illustrates exemplary designs and implant components for tibial trays and inserts for certain embodiments described herein. In particular, this example describes a standard blank tibial tray and insert and a method for altering the standard blanks based on patient-specific data to include a patient-adapted feature (e.g., a patient-adapted tray and insert perimeter that substantially match the perimeter of the patient's resected tibia).

In this example and in certain embodiments, the top surface of the tibial tray can receive a one-piece tibial insert or two-piece tibial inserts. The tibial inserts can include one or more patient-adapted features (e.g., patient-matched or patient-engineered perimeter profile, thickness, and/or joint-facing surface) and/or one or more standard features, in addition to a standard locking mechanism to engage the tibial tray. In certain embodiments the locking mechanism on the tray and insert can include, for example, one or more of: (1) a posterior interlock, (2) a central dovetail interlock, (3) an anterior snap, (4) an anterior interlock, and (5) an anterior wedge.

If desired, the locking mechanism for securing the tibial insert to the tibial tray can be designed and manufactured as an integral portion of the tibial tray. In some embodiments, the locking mechanism can be significantly smaller than the upper surface of the tray, to allow for perimeter matching of the tray, whereby subsequent machining and/or processing of the outer periphery and upper portion of the tibial tray (to patient-matched dimensions) will not significantly degrade or otherwise affect the locking mechanism (i.e., the final patient-matched perimeter of the implant does not cut-into the lock). In an alternative embodiment, the locking mechanism may extend along the entire upper surface of the tibial tray, whereby perimeter matching of the tray results in removal of some portion of the locking mechanism, yet the remainder of the locking mechanism is still capable of retaining the tibial insert on the tibial tray (i.e., the final patient-matched perimeter of the implant cuts into some of the lock structure, but sufficient lock structure remains to retain the insert in the tray). Such embodiments may have locking mechanisms pre-formed in a library of pre-formed tibial tray blanks. As another alternative, one or more locking mechanism designs may be incorporated into the implant design program, with an appropriate locking mechanism design and size chosen at the time of implant design, and ultimately formed into (or otherwise attached to) a tibial tray (chosen or designed to match patient anatomy) during the process of designing, manufacturing and/or modifying the implant for use with the specific patient. Such design files can include CAD files or subroutines of locking mechanism of various sizes, shaped and/or locking features, with an appropriate locking mechanism chosen at an appropriate time. If desired, the design program can ultimately analyze the chosen/designed lock and locking mechanism to confirm that the final lock will be capable of retaining the insert within the tray under loading and fatigue conditions, and alerting (or choosing an alternative design) if FEA or other analyses identifies areas of weakness and/or concern in the currently-chosen design.

Standard blank tibial trays and/or inserts can be prepared in multiple sizes, e.g., having various AP dimensions, ML dimensions, and/or stem and keel dimensions and configurations. For example, in certain-sized embodiments, the stem can be 13 mm in diameter and 40 mm long and the keel can be 3.5 mm wide, 15 degrees biased on the lateral side and 5 degrees biased on the medial side. However, in other-sized embodiments (e.g., having larger or small tray ML and/or AP dimensions, the step and keel can be larger, smaller, or have a different configuration.

As mentioned above, in this example and in certain embodiments, the tibial tray can receive a one-piece tibial insert or two-piece tibial inserts. Alternatively, a two-piece tibial insert can be used with a two-piece tibial tray. Alternatively, a one-piece tibial insert can be used with a two-piece tibial tray.

In various embodiments, single and dual insert systems can be designed to have a different tibial dish depth medially and lateral. The different dish depth can have the same radius or radii medially and laterally or can have different radii. The radii medially and/or laterally can be patient-specific, patient-derived, patient-selected, engineered and/or standard sizes. The dish depth medially and/or laterally can be selected to reflect or replicate at least one of an uncut medial plateau, an uncut lateral tibial plateau, a medial femoral condyle, a lateral femoral condyle, a medial femoral resection level, a lateral femoral resection level, a medial-lateral distal femoral offset, a medial-lateral posterior femoral offset or an engineered offset.

Certain embodiments include altering a blank tibial tray and a blank tibial insert to each include a patient-adapted profile, for example, to substantially match the profile of the patient's resected tibial surface. For example, standard cast tibial tray blanks and standard machined insert blanks (e.g., having standard locking mechanisms) can be finished, e.g., using CAM machining technology, to alter the blanks to include one or more patient-adapted features. The blank tray and insert can be finish machined to match or optimize one or more patient-specific features based on patient-specific data. The patient-adapted features machined into the blanks can include for example, a patient-specific periphery profile and/or one or more medial coronal, medial sagittal, lateral coronal, lateral sagittal bone-facing insert curvatures.

In certain embodiments, the medial and lateral tibial component can, optionally, have the same thickness including composite thickness, but can be implanted at different resection heights. The resection height can be selected as the thickness of the component moved inferiorly, for example in reference from the medial tibial plateau, for the medial side and moved inferiorly, for example in reference from the lateral tibial plateau, for the lateral side. In this manner, the prosthesis can respect or substantially replicate not only the medial but also the lateral location of the joint space preoperatively. Similarly, a single metal backing tibial component can be designed to allow for a medial and a lateral tibial cut that are at different heights in order to replicate the natural medial and/or lateral joint space, in particular when inserts of the same thickness are used. A vertical or oblique connecting cut can then be used from the medial to the lateral side.

The medial and the lateral tibial plateau can also be cut with different slopes for single and dual (medial and separately lateral) components. The slopes can, for example, reflect the medial slope preoperatively on the medial side, the lateral slope preoperatively on the lateral side, or combinations thereof, or one of a medial or one of a lateral slope applied to the contralateral side using a form of offset or mathematical function to modify the slope. Similarly, a single metal backing tibial component can be designed to allow for a medial and a lateral tibial cut that differently slopes in order to replicate the natural medial and/or lateral slope. A vertical or oblique connecting cut can then be used from the medial to the lateral side.

The tibial component can incorporate various combinations of individual features and/or factors to reflect femoral offset and/or the medial or lateral joint line or derivations thereof

Tibial insert geometries can be selected or designed for a patient's tibial shape, medial and/or lateral joint space location or distal or posterior medial and/or lateral femoral offset. Alternatively engineered geometries can be used. In various embodiments, patient selected or designed inserts can be combined with engineered inserts, e.g., a patient-specific medial insert can be combined with an engineered lateral insert. Patient selection or design can be based on a single plane or dimension, e.g., sagittal geometry (optionally matched to tibia or femoral sagittal J-curve) or coronal geometry, on two planes or dimensions, e.g., sagittal and coronal, or can be three-dimensional, for example by also matching, at least in part, a tibial, glenoid or acetabular implant perimeter to the patient's cut bone, for example in a virtual 3D simulation. Insert selection or design can occur using information derived from the patient's native, uncut tibial plateau or other bone shape, e.g., medial or lateral plateau, tibial spines, or combinations thereof. Insert selection or design can also occur using information derived from the patient's femoral shape, e.g., in a sagittal or coronal plane or combinations thereof, or distal or posterior or combinations thereof medial and lateral femoral condyle offset. A medial and a lateral insert height (for single and dual insert components) can be the same, can be greater medially than laterally, or can be greater laterally than medially. A medial side can be concave, flat or convex, engineered, patient selected and/or patient designed. A lateral side can be concave, flat or convex, engineered, patient selected and/or patient designed. Any combination of medial and lateral concave, flat or convex, engineered, patient selected or patient designed insert shapes (single and dual insert designs) is possible. For example, a patient selected or patient designed insert shape can be used medially, while an engineered design is used lateral either in a single or dual insert application. These shape variations may be applicable to tibial components with metal backing and polyethylene inserts as well as any other tibial implant component (known in the art or developed in the future), e.g., using ceramics, composites, all polyethylene tibial components and the like. For any of these embodiments, a height difference between a medial and a lateral tibial bearing surface can be selected or designed based on the patient's native, uncut medial and lateral tibial plateau or based on the patient's medial or lateral distal and/or posterior femoral condyle offset or combinations thereof

Additional embodiments of tibial implant components that are cruciate retaining. Medial and lateral tibial components including trays and inserts or single material designs can be selected or designed to specifically avoid the tibial spines and/or the one or both cruciate ligaments, e.g., the ACL or ACL attachment or the PCL or PCL attachment. The insert perimeter can be optimized to avoid impingement on the MCL or LCL.

The bearing surface of the tibial insert can be adapted to follow the direction of the natural sweep of the femur on the tibia or a desired sweep of the femur on the tibia, both in fixed as well as in mobile bearing designs (slideably or rotatably engageable). The femoral sweep on the tibia can be estimated based on the patient's native, uncut femoral and tibial shape, a wear pattern observed, or kinematic analysis. This adaptation can be achieved by designing or selecting a tibial bearing surface that is, for example, curved medially and straight laterally or curved medially and laterally. The medial and lateral sweep curves can be the same with a single or multiple radii or they can be different with a single or multiple radii. They can be engineered, patient selected, patient derived and/or patient designed. All of these embodiments are applicable to fixed as well as mobile bearings in all joints, e.g. hip, shoulder, ankle, elbow, wrist, hand, foot, knee, spine.

Automated and/or Semi-Automated Optimzation

Any of the methods described herein can be performed, at least in part, using a computer-readable medium having instructions stored thereon, which, when executed by one or more processors, causes the one or more processors to perform one or more operations corresponding to one or more steps in the method. Any of the methods can include the steps of receiving input from a device or user and producing an output for a user, for example, a physician, clinician, technician, or other user. Executed instructions on the computer-readable medium (i.e., a software program) can be used, for example, to receive as input patient-specific information (e.g., images of a patient's biological structure) and provide as output a virtual model of the patient's biological structure. Similarly, executed instructions on a computer-readable medium can be used to receive as input patient-specific information and user-selected and/or weighted parameters and then provide as output to a user values or ranges of values for those parameters and/or for resection cut features, guide tool features, and/or implant component features. For example, in certain embodiments, patient-specific information can be input into a computer software program for selecting and/or designing one or more resection cuts, guide tools, and/or implant components, and one or more of the following parameters can be optimization in the design process: (1) correction of joint deformity; (2) correction of a limb alignment deformity; (3) preservation of bone, cartilage, and/or ligaments at the joint; (4) preservation, restoration, or enhancement of one or more features of the patient's biology, for example, trochlea and trochlear shape; (5) preservation, restoration, or enhancement of joint kinematics, including, for example, ligament function and implant impingement; (6) preservation, restoration, or enhancement of the patient's joint-line location and/or joint gap width; and (7) preservation, restoration, or enhancement of other target features.

Optimization of multiple parameters may result in conflicting constraints; for example, optimizing one parameter may cause an undesired deviation to one or more other parameters. In cases where not all constraints can be achieved at the same time, parameters can be assigned a priority or weight in the software program. The priority or weighting can be automated (e.g., part of the computer program) and/or it can be selected by a user depending on the user's desired design goals, for example, minimization of bone loss, or retention of existing joint-line to preserve kinematics, or combination to accommodate both parameters in overall design. As an illustrative example, in certain embodiments, selection and/or design of a knee implant can include obtaining patient-specific information (e.g., from radiographic images or CT images) of a patient's knee and inputting that information into the computer program to model features such as minimum thickness of femoral component (to minimize resected bone on femur), tibial resection cut height (to minimize resected bone on tibia), and joint-line position (preferably to preserve for natural kinematics). These features can be modeled and analyzed relative to a weighting of parameters such as preserving bone and preserving joint kinematics. As output, one or more resection cut features, guide tool features, and/or implant component features that optimize the identified parameters relative to the selective weightings can be provided.

In any automated process or process step performed by the computer system, constraints pertaining to a specific implant model, to a group of patients or to the individual patient may be taken into account. For example, the maximum implant thickness or allowable positions of implant anchors can depend on the type of implant. The minimum allowable implant thickness can depend on the patient's bone quality.

Any one or more steps of the assessment, selection, and/or design may be partially or fully automated, for example, using a computer-run software program and/or one or more robots. For example, processing of the patient data, the assessment of biological features and/or feature measurements, the assessment of implant component features and/or feature measurements, the optional assessment of resection cut and/or guide tool features and/or feature measurements, the selection and/or design of one or more features of a patient-adapted implant component, the manufacture of the designed/selected implant components and associated guide tools, and/or the implantation procedure(s) may be partially or wholly automated. For example, patient data, with optional user-defined parameters, may be inputted or transferred by a user and/or by electronic transfer into a software-directed computer system that can identify variable implant component features and/or feature measurements and perform operations to generate one or more virtual models and/or implant design specifications, for example, in accordance with one or more target or threshold parameters.

Using Parameters to Assess, Select and/or Design Implants

In various embodiments, an automated and/or semi-automated program can include modules for assessing various parameters described herein, alone or optionally in combination with one or more additional parameters conducted using various formats. For example, the assessment of one or more parameters can be performed in series, in parallel, or in a combination of serial and parallel steps, optionally with a software-directed computer. For example, one or more selected implant component features and feature measurements, optionally with one or more selected resection cut features and feature measurements and one or more selected guide tool features and feature measurements can be altered and assessed in series, in parallel, or in a combination format, to assess the fit between selected parameter thresholds and the selected features and feature measurements. Any one or more of the parameters and features and/or feature measurements can be the first to be selected and/or designed. Alternatively, one or more, or all, of the parameters and/or features can be assessed simultaneously.

The assessment process can be iterative in nature. For example, one or more first parameters can be assessed and the related implant component and/or resection cut features and feature measurements tentatively or conditionally can be determined. Next, one or more second parameters can be assessed and, optionally, one or more features and/or feature measurements determined. Then, the tentative or conditional features and/or feature measurements for the first assessed parameter(s) optionally can be altered based on the assessment and optional determinations for the second assessed parameters. The assessment process can be fully automated or it can be partially automated allowing for user interaction. User interaction can be particularly useful for quality assurance purposes.

In the assessment, different weighting can be applied to any of the parameters or parameter thresholds, for example, based on the patient's age, the surgeon's preference or the patient's preference. Feedback mechanisms can be used to show the user or the software the effect that certain feature and/or feature measurement changes can have on desired changes to parameters values, e.g., relative to selected parameter thresholds. For example, a feedback mechanism can be used to determine the effect that changes in features intended to maximize bone preservation (e.g., implant component thickness(es), bone cut number, cut angles, cut orientations, and related resection cut number, angles, and orientations) have on other parameters such as limb alignment, deformity correction, and/or joint kinematic parameters, for example, relative to selected parameter thresholds. Accordingly, implant component features and/or feature measurements (and, optionally, resection cut and guide tool features and/or feature measurements) can be modeled virtually and modified reiteratively to achieve an optimum solution for a particular patient.

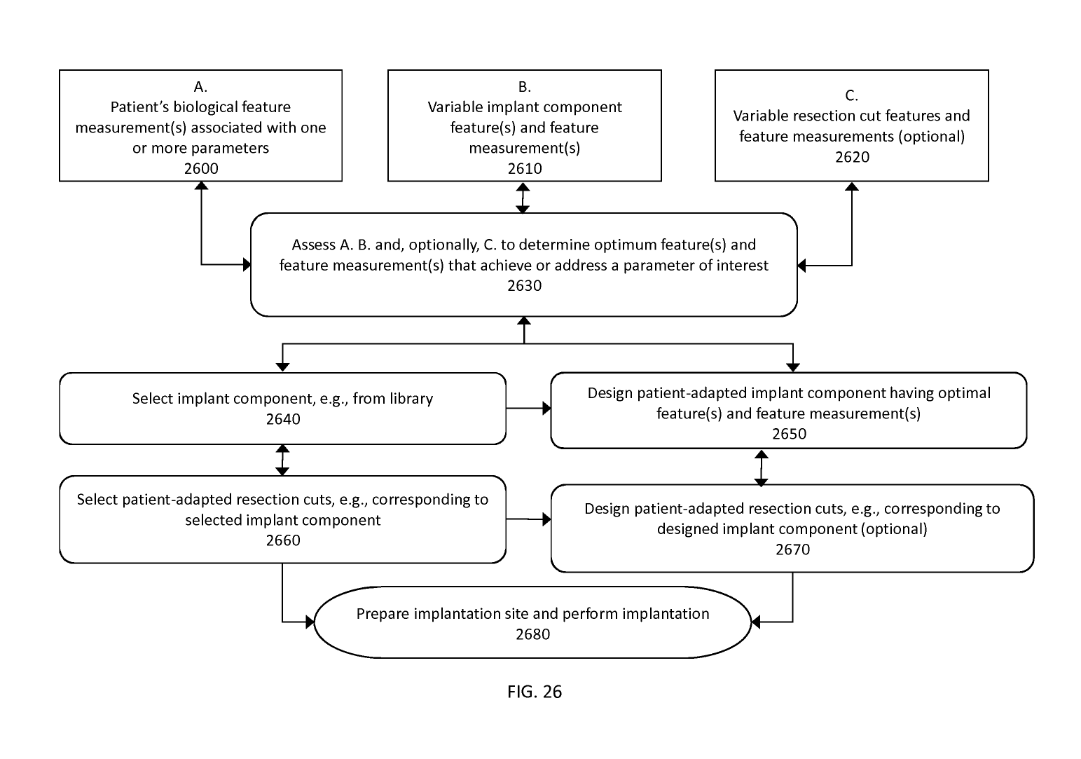

FIG. 26 is a flow chart illustrating the process of assessing and selecting and/or designing one or more implant component features and/or feature measurements, and, optionally assessing and selecting and/or designing one or more resection cut features and feature measurements, for a particular patient. Using the techniques described herein or those suitable and known in the art, one or more of the patient's biological features and/or feature measurements are obtained 2600. In addition, one or more variable implant component features and/or feature measurements are obtained 2610. Optionally, one or more variable resection cut features and/or feature measurements are obtained 2620. Moreover, one or more variable guide tool features and/or feature measurements also can optionally be obtained. Each one of these step can be repeated multiple times, as desired.

The obtained patient's biological features and feature measurements, implant component features and feature measurements, and, optionally, resection cut and/or guide tool features and/or feature measurements then can be assessed to determine the optimum implant component features and/or feature measurements, and optionally, resection cut and/or guide tool features and/or feature measurements, that achieve one or more target or threshold values for parameters of interest 2630 (e.g., by maintaining or restoring a patient's healthy joint feature). This step can be repeated as desired. For example, the assessment step 2630 can be reiteratively repeated after obtaining various feature and feature measurement information 2600, 2610, 2620.

Once the one or more optimum implant component features and/or feature measurements are determined, the implant component(s) can be selected 2640, designed 2650, or selected and designed 2640, 2650. For example, an implant component having some optimum features and/or feature measurements can be designed using one or more CAD software programs or other specialized software to optimize additional features or feature measurements of the implant component. One or more manufacturing techniques described herein or known in the art can be used in the design step to produce the additional optimized features and/or feature measurements. This process can be repeated as desired.

Optionally, one or more resection cut features and/or feature measurements can be selected 2660, designed 2670, or selected and further designed 2660, 2670. For example, a resection cut strategy selected to have some optimum features and/or feature measurements can be designed further using one or more CAD software programs or other specialized software to optimize additional features or measurements of the resection cuts, for example, so that the resected surfaces substantially match optimized bone-facing surfaces of the selected and designed implant component. This process can be repeated as desired.

Moreover, optionally, one or more guide tool features and/or feature measurements can be selected, designed, or selected and further designed. For example, a guide tool having some optimum features and/or feature measurements can be designed further using one or more CAD software programs or other specialized software to optimize additional features or feature measurements of the guide tool. One or more manufacturing techniques described herein or known in the art can be used in the design step to produce the additional, optimized features and/or feature measurements, for example, to facilitate one or more resection cuts that, optionally, substantially match one or more optimized bone-facing surfaces of a selected and designed implant component. This process can be repeated as desired.

As will be appreciated by those of skill in the art, the process of selecting and/or designing an implant component feature and/or feature measurement, resection cut feature and/or feature measurement, and/or guide tool feature and/or feature measurement can be tested against the information obtained regarding the patient's biological features, for example, from one or more MRI or CT or x-ray images from the patient, to ensure that the features and/or feature measurements are optimum with respect to the selected parameter targets or thresholds. Testing can be accomplished by, for example, superimposing the implant image over the image for the patient's joint. In a similar manner, load-bearing measurements and/or virtual simulations thereof may be utilized to optimize or otherwise alter a derived implant design. For example, where a proposed implant for a knee implant has been designed, it may then be virtually inserted into a biomechanical model or otherwise analyzed relative to the load-bearing conditions (or virtually simulations thereof) it may encounter after implantation. These conditions may indicate that one or more features of the implant are undesirable for varying reasons (i.e., the implant design creates unwanted anatomical impingement points, the implant design causes the joint to function in an undesirable fashion, the joint design somehow interferes with surrounding anatomy, the joint design creates a cosmetically-undesirable feature on the repaired limb or skin covering thereof, FEA or other loading analysis of the joint design indicates areas of high material failure risk, FEA or other loading analysis of the joint design indicates areas of high design failure risk, FEA or other loading analysis of the joint design indicates areas of high failure risk of the supporting or surrounding anatomical structures, etc.). In such a case, such undesirable features may be accommodated or otherwise ameliorated by further design iteration and/or modification that might not have been discovered without such analysis relative to the “real world” measurements and/or simulation.

Such load-bearing/modeling analysis may also be used to further optimize or otherwise modify the implant design, such as where the implant analysis indicates that the current design is “over-engineered” in some manner than required to accommodate the patient's biomechanical needs. In such a case, the implant design may be further modified and/or redesigned to more accurately accommodate the patient's needs, which may have an unintended (but potentially highly-desirable) consequence of reducing implant size or thickness, increasing or altering the number of potential implant component materials (due to altered requirements for material strength and/or flexibility), increasing estimate life of the implant, reduce wear or otherwise altering one or more of the various design “constraints” or limitations currently accommodated by the present design features of the implant.

Once optimum features and/or feature measurements for the implant component, and optionally for the resection cuts and/or guide tools, have been selected and/or designed, the implant site can be prepared, for example by removing cartilage and/or resectioning bone from the joint surface, and the implant component can be implanted into the joint 2680.

The joint implant component bone-facing surface, and optionally the resection cuts and guide tools, can be selected and/or designed to include one or more features that achieve an anatomic or near anatomic fit with the existing surface or with a resected surface of the joint. Moreover, the joint implant component joint-facing surface, and optionally the resection cuts and guide tools, can be selected and/or designed, for example, to replicate the patient's existing joint anatomy, to replicate the patient's healthy joint anatomy, to enhance the patient's joint anatomy, and/or to optimize fit with an opposing implant component. Accordingly, both the existing surface of the joint and the desired resulting surface of the joint can be assessed. This technique can be particularly useful for implants that are not anchored into the bone.

As will be appreciated by those of skill in the art, an automated or semi-automated system, either alone or in conjunction with a physician and/or other person, can obtain a measurement of a biological feature (e.g., a target joint) 2600 and then directly select 2640, design, 2650, or select and design 2640, 2650 a joint implant component having desired patient-adapted features and/or feature measurements. Designing can include, for example, design and manufacturing.

### Setting and Weighting Parameters

As described herein, certain embodiments can apply modeling, for example, virtual modeling and/or mathematical modeling, to identify optimum implant component features and measurements, and optionally resection features and measurements, to achieve or advance one or more parameter targets or thresholds. For example, a model of patient's joint or limb can be used to identify, select, and/or design one or more optimum features and/or feature measurements relative to selected parameters for an implant component and, optionally, for corresponding resection cuts and/or guide tools. In certain embodiments, a physician, clinician, or other user can select one or more parameters, parameter thresholds or targets, and/or relative weightings for the parameters included in the model. Alternatively or in addition, clinical data, for example obtained from clinical trials, or intraoperative data, can be included in selecting parameter targets or thresholds, and/or in determining optimum features and/or feature measurements for an implant component, resection cut, and/or guide tool.

One or more parametric thresholds and/or weightings can be applied for the selection and/or designing process. Different parameters can have the same weighting or they can have different weightings. A parameter can include one or more thresholds for selecting one or more implants. The thresholds can include one or more minimum threshold values (e.g., with different weightings), for example, 80%, greater than 80%, 85%, greater than 85%, 90%, greater than 90%, 95%, greater than 95%, 97%, greater than 97%, 98%, greater than 98%, 99%, greater than 99%, 100%, and/or greater than 100% a target value, such as minimum implant coverage of a certain surface on the patient's anatomical structure. Alternatively or in addition, the thresholds can include one or more maximum threshold values (e.g., with different weightings), such as 105%, less than 105%, 103%, less than 103%, 102%, less than 102%, 101%, less than 101%, 100%, and/or less than 100% a target value, such as maximum implant coverage of a certain surface on the patient's anatomical structure.

One or more parameter thresholds can be absolute, for example, by selecting and/or designing for only implants that meet the threshold, for example, a threshold for a particular patient of 95% mediolateral femoral condyle coverage all around the condyle(s). An example of a selection and/or design process having multiple absolute thresholds is a process that selects and/or designs femoral implant components that must meet both a minimum threshold for a particular patient of 95% mediolateral femoral condyle coverage in the central weight-bearing region, and a minimum threshold of greater than 80% mediolateral femoral condyle coverage outside the weight-bearing area.

Alternatively or in addition, one or more parameter thresholds can be contingent on one or more other factors. In particular, a selection and/or designing process can successively search a library for implants, guide tools and/or surgical procedure steps based on contingent thresholds. For example, femoral implant components meeting a minimum threshold of 99% mediolateral femoral condyle coverage initially can be selected. If no implant meets the threshold, or if some implants meet the threshold but do not meet other parameter thresholds, then a second selection round can include implants meeting a minimum threshold of 98% mediolateral femoral condyle coverage. The process can continue to use additional, contingent thresholds until an implant or other comparative feature with the selected parameter thresholds is identified.

Different thresholds can be defined in different anatomic regions and for different parameters. For example, in certain embodiments of a knee implant design, the amount of mediolateral tibial implant component coverage can be set at 90%, while the amount of anteroposterior tibial implant component coverage can be set at 85%. In another illustrative example, the congruency in intercondylar notch shape can be set at 80% required, while the required mediolateral condylar coverage can be set at 95%.

### FEA Analysis

In various alternative embodiments, the automated and/or semi-automated program may conduct FEA or other load analysis on the implant components, such as a tibial tray or guide tool (which may incorporate various patient-specific information, including patient weight and intended activity levels, among other factors), and determine if specific areas of the intended implant design at are an undesirable risk of failure or fatigue. Such areas can be reinforced, thickened or otherwise redesigned (if desired) to accommodate and/or alleviate such risks (desirably before actual manufacture of the implant). In a similar manner, areas of lower stress/fracture risk can be redesigned (if desired) by removal of material, etc., which may improve the fit and/or performance of the implant in various ways. Of course, either or both of the upper and lower surface of the tibial tray may be processed and/or redesigned in this manner.

### Blanks

In various embodiments, implant components may be constructed as a “standard” or “blank” in various sizes and may be subsequently modified and/or otherwise specifically formed for each patient based on their imaging data and anatomy. Computer modeling may be used and a library of virtual standards may be created for each of the components. A library of physical standards may also be amassed for each of the components. As part of the design process, the automated and/or semi-automated program may include information regarding the availability of blanks, as well as relevant sizes and/or shapes thereof, as an additional criteria in the design and/or selection of implant component and surgical procedures.

Any of the implant components for a knee, hip, ankle, shoulder, elbow or wrist or other joint can be formed or adapted based on a pre-existing blank. Various dimensions or shapes of the joint can be determined and a pre-existing blank component can then be selected and the shape adapted to the patient's shape, for example, by selectively removing material, e.g., with a machining or cutting or abrasion or other process, or by adding material. The shape of the blank will generally be selected to be smaller than the target anatomy when material is added to achieve the patient adapted or patient specific implant features or surfaces. The shape of the blank will generally be selected to be larger than the target anatomy when material is removed to achieve the patient adapted or patient specific implant features or surfaces. Any manufacturing process known in the art or developed in the future can be used to add or remove material, including for metals, ceramics, plastics and other materials.

An outer, bone facing component can be adapted to or matched to the patient's anatomic features using a blank in this manner. Alternatively or additionally, an insert can be adapted or shaped based on the patient's anatomic features in one or two or three dimensions. For example, a standard insert, e.g., with a standard locking mechanism into the outer component, can be adapted so that its outer rim will not overhang the patient's anatomy, e.g., a glenoid rim, before or after a surgical alteration such as a cutting or reaming. The surgical alteration can, in this example as well as in many of the foregoing and following embodiments, be simulated on a computer and the insert blank can then be shaped based on the result of the simulation. Thus, a glenoid insert as well as a metal backing can be adapted, e.g., machined, so that its perimeter will match the glenoid rim in at least a portion either before or after the surgical alteration of the glenoid. Similar adaptations are possible in any other joint, including the hip, knee, ankle, elbow and wrist.

In various embodiments, the position and orientation of any peg, keel or other fixation features of acetabular or glenoid or femoral or tibial components or implant components in any other joint can be designed, adapted, shaped, changed or optimized relative to the patient's geometry, e.g., relative to the adjacent cortex or, for example, the center of a medullary cavity or other anatomic or geometric features. In a glenoid, the length and width of the attachment mechanisms can be adapted to the mediolateral width of the glenoid or to the existing bone stock available or any other glenoid dimension, e.g., superoinferior. In a hip, the length and width of the attachment mechanisms can be adapted to the thickness of the acetabular wall or to the existing bone stock available in the underlying and adjacent bone structures including the acetabular roof. In a knee, the position of pegs or keels or stems can be standard or can be patient specific or adapted based on the patient's anatomy.

### Standard Features

In various embodiments, the automated and/or semi-automated system may design and/or select one or more standard features for inclusion into an implant design, such as a standard locking mechanisms to secure a tibial insert into a tibial tray. The locking mechanism can be pre-configured and/or available, for example, in two or three different geometries or size. Optionally, the automated and/or semi-automated program can have a library of CAD files or subroutines with different sizes and geometries of locking mechanisms available. For example, in a first step, the computer program can define, design or select a tibial, acetabular or glenoid implant profile that best matches a patient's cut (or, optionally, uncut) tibia, acetabulum or glenoid. In a second step, the computer program can then select the pre-configured CAD file or subroutine that is best suited for a given tibial or acetabular or glenoid perimeter or other shape or location or size. Moreover, the type of locking mechanism (e.g., snap, dovetail etc.) can be selected based on patient specific parameters, e.g., body weight, height, gender, race, activity level etc.).

Other standard features can include features that attach implant components to the underlying bone. Any attachment mechanism known in the art can be used, e.g., pegs, fins, keels, stems, anchors, pins and the like. The attachment mechanisms can be standard in at least one of shape, size and location. Thus, in a glenoid component, an all polyethylene component can be used. Using imaging data, the blank glenoid component can be aligned relative to the patient's glenoid (optionally after a simulated surgical intervention) to optimize the position of any standard attachment mechanisms relative to the bone to which they are intended to be attached. Once the optimal position of the glenoid blank and its attachment mechanisms has been selected, the outer rim and, optionally, the bearing surface of the component can be adapted based on the patient's anatomy. Thus, for example, the outer periphery of the implant can be machined then to substantially align with portions of the patients glenoid rim.

### Manufacturing

The step of designing an implant component and/or guide tool as described herein can include both configuring one or more features, measurements, and/or dimensions of the implant and/or guide tool (e.g., derived from patient-specific data from a particular patient and adapted for the particular patient) and manufacturing the implant. In certain embodiments, manufacturing can include making the implant component and/or guide tool from starting materials, for example, metals and/or polymers or other materials in solid (e.g., powders or blocks) or liquid form. In addition or alternatively, in certain embodiments, manufacturing can include altering (e.g., machining) an existing implant component and/or guide tool, for example, a standard blank implant component and/or guide tool or an existing implant component and/or guide tool (e.g., selected from a library). The manufacturing techniques to making or altering an implant component and/or guide tool can include any techniques known in the art today and in the future. Such techniques include, but are not limited to additive as well as subtractive methods, i.e., methods that add material, for example to a standard blank, and methods that remove material, for example from a standard blank.

In various embodiments described herein, the act of designing an implant component can include manufacturing the implant component having the related design features. For example, designing an implant component can include preoperatively establishing a design of one or more features of an implant component, for example, using a CAD computer program on a computer system specialized operated for such use and having one or more user interfaces, and instructing the transfer of that design data, for example, from a CAD computer program or computer system to a CAM (computer-aided manufacturing) computer program or computer system. Optionally, in certain embodiments, designing the implant can further include instructing the initiation of manufacturing the physical implant and/or manufacturing the implant.

Various technologies appropriate for this purpose are known in the art, for example, as described in Wohlers Report 2009, State of the Industry Annual Worldwide Progress Report on Additive Manufacturing, Wohlers Associates, 2009 (ISBN 0-9754429-5-3), available from the web www.wohlersassociates.com; Pham and Dimov, Rapid manufacturing, Springer-Verlag, 2001 (ISBN 1-85233-360-X); Grenda, Printing the Future, The 3D Printing and Rapid Prototyping Source Book, Castle Island Co., 2009; Virtual Prototyping & Bio Manufacturing in Medical Applications, Bidanda and Bartolo (Eds.), Springer, Dec. 17, 2007 (ISBN: 10: 0387334297; 13: 978-0387334295); Bio-Materials and Prototyping Applications in Medicine, Bártolo and Bidanda (Eds.), Springer, Dec. 10, 2007 (ISBN: 10: 0387476822; 13: 978-0387476827); Liou, Rapid Prototyping and Engineering Applications: A Toolbox for Prototype Development, CRC, Sep. 26, 2007 (ISBN: 10: 0849334098; 13: 978-0849334092); Advanced Manufacturing Technology for Medical Applications: Reverse Engineering, Software Conversion and Rapid Prototyping, Gibson (Ed.), Wiley, January 2006 (ISBN: 10: 0470016884; 13: 978-0470016886); and Branner et al., “Coupled Field Simulation in Additive Layer Manufacturing,” 3rd International Conference PMI, 2008 (10 pages).

Exemplary techniques for adapting an implant to a patient's anatomy include, but are not limited to those shown in Table 9.

TABLE 9
Exemplary techniques for forming or altering a patient-specific and/or patient-
engineered implant component for a patient's anatomy

| Technique | Brief description of technique and related notes |
|---|---|
| CNC | CNC refers to computer numerically controlled (CNC) machine |
| | tools, a computer-driven technique, e.g., computer-code |
| | instructions, in which machine tools are driven by one or more |
| | computers. Embodiments of this method can interface with |
| | CAD software to streamline the automated design and |
| | manufacturing process. |
| CAM | CAM refers to computer-aided manufacturing (CAM) and can |
| | be used to describe the use of software programming tools to |
| | efficiently manage manufacturing and production of products |
| | and prototypes. CAM can be used with CAD to generate CNC |
| | code for manufacturing three-dimensional objects. |
| Casting, including | Casting is a manufacturing technique that employs a mold. |
| casting using rapid | Typically, a mold includes the negative of the desired shape of |
| prototyped casting | a product. A liquid material is poured into the mold and |
| patterns | allowed to cure, for example, with time, cooling, and/or with |
| | the addition of a solidifying agent. The resulting solid material |
| | or casting can be worked subsequently, for example, by sanding |
| | or bonding to another casting to generate a final product. |
| Welding | Welding is a manufacturing technique in which two |
| | components are fused together at one or more locations. In |
| | certain embodiments, the component joining surfaces include |
| | metal or thermoplastic and heat is administered as part of the |
| | fusion technique. |
| Forging | Forging is a manufacturing technique in which a product or |
| | component, typically a metal, is shaped, typically by heating |
| | and applying force. |
| Rapid prototyping | Rapid prototyping refers generally to automated construction of |
| | a prototype or product, typically using an additive |
| | manufacturing technology, such as EBM, SLS, SLM, SLA, |
| | DMLS, 3DP, FDM and other technologies |
| EBM ® | EBM ® refers to electron beam melting (EBM ®), which is a |
| | powder-based additive manufacturing technology. Typically, |
| | successive layers of metal powder are deposited and melted |
| | with an electron beam in a vacuum. |
| SLS | SLS refers to selective laser sintering (SLS), which is a powder- |
| | based additive manufacturing technology. Typically, |
| | successive layers of a powder (e.g., polymer, metal, sand, or |
| | other material) are deposited and melted with a scanning laser, |
| | for example, a carbon dioxide laser. |
| SLM | SLM refers to selective laser melting ™ (SLM), which is a |
| | technology similar to SLS; however, with SLM the powder |
| | material is fully melted to form a fully-dense product. |
| SLA or SL | SLA or SL refers to stereolithography (SLA or SL), which is a |
| | liquid-based additive manufacturing technology. Typically, |
| | successive layers of a liquid resin are exposed to a curing, for |
| | example, with UV laser light, to solidify each layer and bond it |
| | to the layer below. This technology typically requires the |
| | additional and removal of support structures when creating |
| | particular geometries. |
| DMLS | DMLS refers to direct metal laser sintering (DMLS), which is a |
| | powder-based additive manufacturing technology. Typically, |
| | metal powder is deposited and melted locally using a fiber optic |
| | laser. Complex and highly accurate geometries can be |
| | produced with this technology. This technology supports net- |
| | shaping, which means that the product generated from the |
| | technology requires little or no subsequent surface finishing. |
| LC | LC refers to LaserCusing ® (LC), which is a powder-based |
| | additive manufacturing technology. LC is similar to DMLS; |
| | however, with LC a high-energy laser is used to completely |
| | melt the powder, thereby creating a fully-dense product. |
| 3DP | 3DP refers to three-dimensional printing (3DP), which is a |
| | high-speed additive manufacturing technology that can deposit |
| | various types of materials in powder, liquid, or granular form in |
| | a printer-like fashion. Deposited layers can be cured layer by |
| | layer or, alternatively, for granular deposition, an intervening |
| | adhesive step can be used to secure layered granules together in |
| | bed of granules and the multiple layers subsequently can be |
| | cured together, for example, with laser or light curing. |
| LENS | LENS ® refers to Laser Engineered Net Shaping ™ (LENS ®), |
| | which is a powder-based additive manufacturing technology. |
| | Typically, a metal powder is supplied to the focus of the laser |
| | beam at a deposition head. The laser beam melts the powder as |
| | it is applied, in raster fashion. The process continues layer by |
| | and layer and requires no subsequent curing. This technology |
| | supports net-shaping, which means that the product generated |
| | from the technology requires little or no subsequent surface finishing. |
| FDM | FDM refers to fused deposition modeling ™ (FDM) is an |
| | extrusion-based additive manufacturing technology. Typically, |
| | beads of heated extruded polymers are deposited row by row |
| | and layer by layer. The beads harden as the extruded polymer cools. |

### Results of Different Manufacturing Methods

Implant components generated by different techniques can be assessed and compared for their accuracy of shape relative to the intended shape design, for their mechanical strength, and for other factors. In this way, different manufacturing techniques can supply another consideration for achieving an implant component design with one or more target features. For example, if accuracy of shape relative to the intended shape design is critical to a particular patient's implant component design, then the manufacturing technique supplying the most accurate shape can be selected. If a minimum implant thickness is critical to a particular patient's implant component design, then the manufacturing technique supplying the highest mechanical strength and therefore allowing the most minimal implant component thickness, can be selected. Branner et al. describe a method a method for the design and optimization of additive layer manufacturing through a numerical coupled-field simulation, based on the finite element analysis (FEA). Branner's method can be used for assessing and comparing product mechanical strength generated by different additive layer manufacturing techniques, for example, SLM, DMLS, and LC.

In certain embodiments, an implant can include components and/or implant component parts produced via various methods. For example, a knee implant can include a metal femoral implant component produced by casting or by an additive manufacturing technique that is patient-specific with respect to a particular patient's M-L dimension and standard with respect to the patient's femoral intercondylar distance; a tibial component cut from a blank and machined to be patient-specific for the perimeter of the patient's cut tibia; and a tibial insert having a standard lock and a top surface that includes a standard intercondylar distance between the tibial insert dishes to accommodate the standard femoral intercondylar distance of the femoral implant.

### Repair Materials

A wide variety of materials find use in the practice of the embodiments described herein, including, but not limited to, plastics, metals, crystal free metals, ceramics, biological materials (e.g., collagen or other extracellular matrix materials), hydroxyapatite, cells (e.g., stem cells, chondrocyte cells or the like), or combinations thereof. Based on the information (e.g., measurements) obtained regarding the defect and the articular surface and/or the subchondral bone, a repair material can be formed or selected. Further, using one or more of these techniques described herein, a cartilage replacement or regenerating material having a curvature that will fit into a particular cartilage defect, will follow the contour and shape of the articular surface, and will match the thickness of the surrounding cartilage. The repair material can include any combination of materials, and typically includes at least one non-pliable material, for example materials that are not easily bent or changed.

Currently, joint repair systems often employ metal and/or polymeric materials in prostheses which are anchored into the underlying bone. A wide-variety of metals is useful in the practice of the embodiments described herein, and can be selected based on any criteria. For example, material selection can be based on resiliency to impart a desired degree of rigidity. Non-limiting examples of suitable metals include silver, gold, platinum, palladium, iridium, copper, tin, lead, antimony, bismuth, zinc, titanium, cobalt, stainless steel, nickel, iron alloys, cobalt alloys, such as Elgiloy®, a cobalt-chromium-nickel alloy, and MP35N, a nickel-cobalt-chromiummolybdenum alloy, and Nitinol T™, a nickel-titanium alloy, aluminum, manganese, iron, tantalum, crystal free metals, such as Liquidmetal® alloys (available from LiquidMetal Technologies, www.liquidmetal.com), other metals that can slowly form polyvalent metal ions, for example to inhibit calcification of implanted substrates in contact with a patient's bodily fluids or tissues, and combinations thereof

Suitable synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Bioresorbable synthetic polymers can also be used such as dextran, hydroxyethyl starch, derivatives of gelatin, polyvinylpyrrolidone, polyvinyl alcohol, poly[N-(2-hydroxypropyl) methacrylamide], poly(hydroxy acids), poly(epsilon-caprolactone), polylactic acid, polyglycolic acid, poly(dimethyl glycolic acid), poly(hydroxy butyrate), and similar copolymers.

Other appropriate materials include, for example, the polyketone known as polyetheretherketone (PEEK). This includes the material PEEK 450G, which is an unfilled PEEK approved for medical implantation available from Victrex of Lancashire, Great Britain. (Victrex is located at www.matweb.com or see Boedeker www.boedeker.com). Other sources of this material include Gharda located in Panoli, India (www.ghardapolymers.com).

It should be noted that the material selected can also be filled. For example, other grades of PEEK are also available and contemplated, such as 30% glass-filled or 30% carbon filled, provided such materials are cleared for use in implantable devices by the FDA, or other regulatory body. Glass filled PEEK reduces the expansion rate and increases the flexural modulus of PEEK relative to that portion which is unfilled. The resulting product is known to be ideal for improved strength, stiffness, or stability. Carbon filled PEEK is known to enhance the compressive strength and stiffness of PEEK and lower its expansion rate. Carbon filled PEEK offers wear resistance and load carrying capability.

As will be appreciated, other suitable similarly biocompatible thermoplastic or thermoplastic polycondensate materials that resist fatigue, have good memory, are flexible, are deflectable, have very low moisture absorption, and/or have good wear and/or abrasion resistance, can be used. The implant can also be comprised of polyetherketoneketone (PEKK). Other materials that can be used include polyetherketone (PEK), polyetherketoneetherketoneketone (PEKEKK), and polyetheretherketoneketone (PEEKK), and, generally, a polyaryletheretherketone. Further, other polyketones can be used as well as other thermoplastics.

Polymers can be prepared by any of a variety of approaches including conventional polymer processing methods. Preferred approaches include, for example, injection molding, which is suitable for the production of polymer components with significant structural features, and rapid prototyping approaches, such as reaction injection molding and stereo-lithography. The substrate can be textured or made porous by either physical abrasion or chemical alteration to facilitate incorporation of the metal coating. Other processes are also appropriate, such as extrusion, injection, compression molding and/or machining techniques. Typically, the polymer is chosen for its physical and mechanical properties and is suitable for carrying and spreading the physical load between the joint surfaces.

More than one metal and/or polymer can be used in combination with each other. For example, one or more metal-containing substrates can be coated with polymers in one or more regions or, alternatively, one or more polymer-containing substrate can be coated in one or more regions with one or more metals.

The system or prosthesis can be porous or porous coated. The porous surface components can be made of various materials including metals, ceramics, and polymers. These surface components can, in turn, be secured by various means to a multitude of structural cores formed of various metals. Suitable porous coatings include, but are not limited to, metal, ceramic, polymeric (e.g., biologically neutral elastomers such as silicone rubber, polyethylene terephthalate and/or combinations thereof or combinations thereof. There can be more than one coating layer and the layers can have the same or different porosities.

The coating can be applied by surrounding a core with powdered polymer and heating until cured to form a coating with an internal network of interconnected pores. The tortuosity of the pores (e.g., a measure of length to diameter of the paths through the pores) can be important in evaluating the probable success of such a coating in use on a prosthetic device. The porous coating can be applied in the form of a powder and the article as a whole subjected to an elevated temperature that bonds the powder to the substrate. Selection of suitable polymers and/or powder coatings can be determined in view of the teachings and references cited herein, for example based on the melt index of each.

Any material known in the art can be used for any of the implant systems and component described in the foregoing embodiments, for example including, but not limited to metal, metal alloys, combinations of metals, plastic, polyethylene, cross-linked polyethylene's or polymers or plastics, pyrolytic carbon, nanotubes and carbons, as well as biologic materials.

### Automated Surgical Systems, Robots and Procedures

In various embodiments, automated systems, devices, and/or methods can be deployed intraoperatively and utilized to prepare a surgical implantation site and/or place a patient-adapted implant. Automated systems, devices, and methods may be fully-automated and/or semi-automated and may include, or include the use of, one or more robots, robotic surgical systems, robotic surgical tools/equipment, computer-controlled surgical systems, computer-controlled surgical tools/equipment, robotic control systems, and computing systems.

Various robotic surgical systems are commercially available, including the RIO robotic-arm interactive orthopedic system (MAKO Surgical, Ft. Lauderdale, Fla.), the da Vinci Surgical System (Intuitive Surgical, Sunnyvale, Calif.) the Magellan Robotic System (Hansen Medical, Mountain View, Calif.) and the Hansen Sensei Robot Surgical System (Texas Cardiac Arrhythmia Institute, Austin, Tex.). These systems typically incorporate a manipulator arm or arms in combination with an electronic computer controller, and the majority of such systems include sensors and registration features to align the system with an intended environment of use. Robotic surgical systems can be supervisory-controlled systems (i.e., the robot performs the surgery based on a prepared surgical plan), tele-surgical systems (i.e., where the robot is directed by a surgeon from a remote location), or shared-control system (i.e., where the surgeon and robot share surgical tasks or functions).

The RIO™ Robotic Arm Interactive Orthopedic System is a proprietary robotic arm system that enables the surgeon to pre-operatively plan the alignment and placement of knee resurfacing implants and to intra-operatively sculpt complex, anatomic, tissue-sparing and bone-conserving cuts. RIO™ assists the surgeon by executing a pre-planned surgical procedure while allowing for real-time intra-operative adjustments for correct knee kinematics and soft-tissue balance. The RIO system provides the surgeon with real-time visual, tactile and auditory feedback to desirably facilitate optimal joint resurfacing and implant positioning.

The da Vinci Surgical System is a tele-surgical system that enables surgeons to perform delicate and complex operations through a few tiny incisions with increased vision, precision, dexterity and control. The da Vinci Surgical System consists of several components, including an ergonomically designed console where the surgeon sits while operating, a patient-side cart where the patient lays during surgery, four interactive robotic arms, a high-definition 3D vision system and proprietary EndoWrist® instruments. In using the system, the surgeon's fingers grasp the master controls below the display with hands and wrists naturally positioned relative to this surgeon's eyes, and the system translates the surgeon's hand, wrist and finger movements into precise, real-time movements of surgical instruments. The robotic arms move around fixed pivot points, which desirably reduces patient trauma, improves the cosmetic outcome, and increases overall precision. The system requires that every surgical maneuver be under the direct control of the surgeon. Repeated safety checks prevent any independent movement of the instruments or robotic arms.

The Magellan™ Robotic System is a tele-surgical system that cannulates peripheral vessels from a centralized, remote workstation using a proprietary technology that delivers simultaneous distal tip control of a catheter and a sheath. This system is designed to provide a robotically stabilized conduit for the placement and delivery of therapeutic devices during peripheral vascular intervention. The system desirably provides vessel navigation with less trauma and more controllability than current manual approaches, and isolates the physician from radiation exposure and procedural fatigue. In addition, the system provides precise robotic control of distal catheter tips during the interventional procedure.

The Hansen Sensei Robot Surgical System is a robotic system for the accurate and stable control of catheter movement during complex cardiac procedures performed to diagnose and treat patients suffering from cardiac arrhythmias. The system manipulates a magnetic field created around the patient in order to guide catheters with magnetic tips. The system is controlled by a physician who maneuvers the catheters remotely using touch screens and a joy stick. The system claims to increase the accuracy of catheter placement, improve procedure safety and enhance patient outcomes.

Various embodiments of the present invention disclose and describe robotic surgical systems and methods that can be utilized to assist with the planning, design and execution of a joint replacement/resurfacing procedure, with or without surgeon intervention, including the design, selection and/or manufacturing of the joint replacement/resurfacing implant. The robot or other surgical tool can directly execute the surgical plan or, alternatively, it can guide a surgeon in executing the surgical plan, for example via haptic feedback or by limiting the excursion of a surgical instrument held by the surgeon in one or two or three dimensions. The system can also act as a surgical assistant and/or “back table” technician, by holding or otherwise “parking” various tools/instruments and/or implants in desired locations relative to the surgical site, either within the surgical site or adjacent to the site in a sterile condition and/or location.

In the various disclosed embodiments, one or more preoperative image scans are taken of an intended surgical site, including anatomic and/or biomechanical data of the patient. Based on this anatomic or biomechanical data, including for example one or more anatomic or biomechanical axis, a surgical plan can be developed for partial or complete execution by the robotic surgical system. The anatomic and biomechanical data can, for example, be obtained at least in part using a scan such as ultrasound (2D and 3D), laser imaging, optical imaging, conventional or digital radiography, digital tomosynthesis, cone beam CT, conventional CT scanning, spiral CT, MRI with 2D or 3D sequences and any other imaging technique know today or developed in the future. The data can be manipulated and/or processed in various fashions, and can be utilized to create a 2D or 3D electronic or physical model of the patient's anatomy for use in further steps of the invention.

In addition, various embodiments of the present invention will further include data on soft tissues and/or other intervening tissues. Moreover, data regarding the patient's disease state, body type, weight, sex, lifestyle, intended implant use, BMI, etc., can be included in the data considered and evaluated by the system.

### Implant Design First Approach

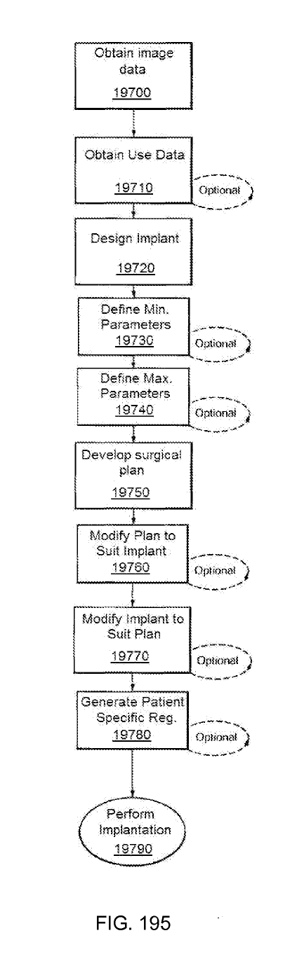

In one embodiment of the present invention (see FIG. 195), the robotic control system (or other computing system) initially utilizes the anatomic/biomechanical data 19700 and 19710 and/or the electronic/physical model to design, select, adapt and/or modify an implant 19720 for use in the targeted anatomical site. Such sites can include a femur (femoral condyle, trochlea, etc.), a tibia, an acetabulum, a femoral neck or head, a glenoid, a humeral head, and/or a vertebrae. The system desirably selects, adapts and/or designs an implant having structural and/or performance features appropriate to the targeted anatomical site and patient. Such features can include a suitable implant size and/or geometry, as well as a desired or appropriate minimum implant thickness or other features 19730 in one or more locations appropriate to anticipated loading conditions due to patient size, geometry, weight, implant material properties, implant component characteristics, etc. In addition, the features can include a desired or appropriate maximum implant thickness or other features 19740 in one or more locations to accommodate the native joint geometry and/or soft/connective tissue conditions (i.e., tightness, laxity, scarification, etc.) as well as accommodating the existing underlying bone stock and/or biomechanical loading conditions on the underlying bone. A significant factor can also include a desire to minimize the removal of supporting anatomical structures, if possible. The surgical plan will desirably consider a minimum or maximum implant thickness and adapt the surgical procedure accordingly, e.g., adjust the depth of burring on a femoral condyle or in a hip. The minimum thickness of the implant can be selected or designed based on material properties of the implant, loading conditions, types of implant components used, patient shape, weight, activity level etc. The maximum thickness of the implant can be a function of the underlying bone stock, e.g., the amount of bone that the surgeon can reasonably remove intraoperatively without impairing the biomechanical strength of the bone or joint.

After the initial implant design/selection step has been accomplished, the system subsequently develops a surgical plan 19750 for the robotic-assisted surgical approach and procedure, taking into account the anatomical site data and/or geometry of the anatomical site, the geometry, size and thickness of the implant and its intended implantation position, the amount of underlying bone stock, the amount and direction of required preparation of the anatomical surfaces, the biomechanical conditions and/or the alignment and/or intended correction of alignment of the joint. Also considered are the type of procedure (i.e., open, partially-open and/or minimally-invasive), the amounts and positions of intervening tissues, the size and orientation of available access paths (i.e., smaller incision will mean smaller access path and/or heavier patient with larger fat deposits may increase depth of access “tunnel” thereby potentially limiting maneuvering room for surgical tools).

Once a desired surgical plan has been created, the plan can be reviewed and/or revised/improved (if desired) using information and/or parameters from the implant design/selection phase 19760. Revisions/redesign and/or reselection of an implant may be appropriate or desirous where the surgical plan is suboptimal in one or more respects, or simply as a means of ensuring that the surgical procedure and chosen implant are optimized for the patient. For example, where the embodiment design/selects an appropriate implant, but the surgical plan indicates a difficult or otherwise suboptimal plan for implantation, it may be desirous to choose/design a different implant appropriate for the chosen surgical plan 19770, or modifying the implant in some manner to widen or otherwise alter the available surgical plan options. In such a case, the final implant/plan choice may be suboptimal in one or more respects (i.e., the implant is not the absolute “best” implant for the patient, but is an acceptable alternative), but can be implanted in a minimally-invasive manner, resulting in shorter healing times and less scarification of the anatomy. Similarly, the chosen procedure may be altered due to implant factors that cannot be ignored or modified.

After the implant and surgical plan have been designed/chosen, the implant can be designed, manufactured, selected and/or modified as appropriate.

The robotic system can be utilized to execute the appropriate surgical plan 19790. As previously noted, the robot can be utilized to directly execute the entire surgical plan, or portions thereof (for example, preparing an individual implantation site in preparation for a joint implant) or the system can assist and/or “guide” the surgeon in executing the surgical plan (i.e., providing tools as needed, executing individual surgical steps including cutting or burring operations to desired depths and/or along desired cutting planes and/or displaying pertinent surgical steps prior to or as they occur). If required, the system could also monitor the procedure and identify incorrect surgical steps and/or highlight areas of concern (i.e., where actual anatomical conditions are different than those anticipated from the imaging data). If desired, the system could include ongoing feedback and/or checksum operations to identify procedure steps, and could include an “on the fly” analysis subroutine that identifies and recommends improvements or changes to the surgical procedure based on current conditions. If desired, the system could include optical recognition software to cross-reference visual information against the imaged anatomical data, and possibly display the anatomy with identifiers and/or other indicia to assist the surgeon with the procedure.

### Surgical Plan First Approach

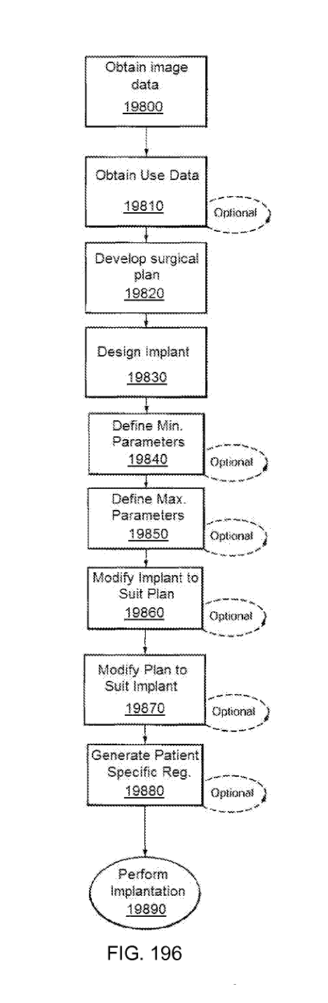

In one alternative embodiment (see FIG. 196), the robotic control system (or other computing system) initially utilizes the anatomic/biomechanical data 19800 and 19810 and/or the electronic/physical model to develop a surgical plan 19820 for the robotic-assisted surgical approach and procedure, taking into account the anatomical site data and/or geometry of the anatomical site, the amount of underlying bone stock, the amount and direction of required preparation of the anatomical surfaces, the biomechanical conditions and/or the alignment and/or intended correction of alignment of the joint. Also considered are the type of procedure (i.e., open, partially-open and/or minimally-invasive) and the amounts and positions of intervening tissues, the size and orientation of available access paths (i.e., smaller incision will mean smaller access path and/or heavier patient with larger fat deposits may increase depth of access “tunnel,” thereby potentially limiting maneuvering room for surgical tools). If desired, an estimated implant shape/size may be incorporated into the initial analysis, or the system can derive an anatomical model of the articulating surfaces from the anatomical data.

Once a desired surgical plan has been created, the system can utilize the intended plan and the anatomic/biomechanical data and/or the electronic/physical model to design, select, adapt and/or modify an implant for use in the targeted anatomical site 19830. Such sites can include a femur (femoral condyle, trochlea, etc.), a tibia, an acetabulum, a femoral neck or head, a glenoid, a humeral head, and/or a vertebrae. The system desirably designs/chooses an implant having structural and/or performance features appropriate to the targeted anatomical site and patient, including a desired constraint of being within size and/or shape that can be accommodated by the intended surgical plan. Additional design features can include a suitable implant size and/or geometry, as well as a desired or appropriate minimum implant thickness or other features 19840 in one or more locations appropriate to anticipated loading conditions due to patient size, geometry, weight, implant material properties, implant component characteristics, etc. In addition, the features can include a desired or appropriate maximum implant thickness or other features 19850 in one or more locations to accommodate the native joint geometry and/or soft/connective tissue condition (i.e., tightness, laxity, scarification, etc.) as well as accommodating the existing underlying bone stock and/or biomechanical loading conditions on the underlying bone. A significant factor can also include a desire to minimize the removal of supporting anatomical structures, if possible. The surgical plan will desirably consider a minimum or maximum implant thickness and adapt the surgical procedure accordingly, e.g., adjust the depth of burring on a femoral condyle or in a hip. The minimum thickness of the implant can be selected or designed based on material properties of the implant, loading conditions, types of implant components used, patient shape, weight, activity level, etc. The maximum thickness of the implant can be a function of the underlying bone stock, e.g., the amount of bone that the surgeon can reasonably remove intraoperatively without impairing the biomechanical strength of the bone or joint.

Once a desired implant has been designed/selected/adapted, the implant features can be reviewed and/or revised/improved (if desired) or further adapted using information and/or parameters from the intended surgical planning phase 19860. Revisions/redesign, readaptation and/or reselection of the surgical plan may also be appropriate or desirous where the implant is suboptimal in one or more respects, or simply as a means of ensuring that the surgical procedure and chosen implant are optimized for the patient 19870. For example, where the embodiment designs an appropriate surgical plan, but the surgical plan cannot accommodate the designed/selected implant (or the selected implant is inadequate or otherwise suboptimal for implantation), it may be desirous to choose/design a different plan appropriate for the implant, or modifying the plan in some manner to widen or otherwise alter the available surgical implant options. In such a case, the final implant/plan choice may be suboptimal in one or more respects (i.e., the implant is not the absolute “best” implant for the patient, but is an acceptable alternative), but can be implanted in a partially-open manner, resulting in better performing kinematics and/or durability. Similarly, the chosen implant may be altered due to surgical plan factors that cannot be ignored or modified.

After the implant and surgical plan have been designed/chosen,the implant can be designed, manufactured, selected and/or modified as appropriate.

The robotic system can be utilized to execute the appropriate surgical plan 19890. As previously noted, the robot can be utilized to directly execute the entire surgical plan, or portions thereof (for example, preparing an individual implantation site in preparation for a joint implant) or the system can assist and/or “guide” the surgeon in executing the surgical plan (i.e., providing tools as needed, executing individual surgical steps including cutting or burring operations to desired depths and/or along desired cutting planes and/or displaying pertinent surgical steps prior to or as they occur). If required, the system could also monitor the procedure and identify incorrect surgical steps and/or highlight areas of concern (i.e., where actual anatomical conditions are different than those anticipated from the imaging data). If desired, the system could include ongoing feedback and/or checksum operations to identify procedure steps, and could include an “on the fly” analysis subroutine that identifies and recommends improvements or changes to the surgical procedure based on current conditions. If desired, the system could include optical recognition software to cross-reference visual information against the imaged anatomical data, and possibly display the anatomy with identifiers and/or other indicia to assist the surgeon with the procedure.

If the surgical plan is determined first, the implant thickness can, for example, be adapted for that surgical plan or it can be fixed or it can include a minimum or a maximum. If the implant thickness is adapted for a given surgical plan in a patient, the adaption of the implant thickness can include lower and upper boundaries, e.g., a lower boundary to protect against fatigue fracture. If a surgical plan would require use of an implant thickness below the lower boundary, it can be optionally rejected and replaced by a surgical plan that will respect such lower boundary. This process can be performed in an iterative fashion. If a surgical plan would require use of an implant thickness above the upper boundary, it can be optionally rejected and replaced by a surgical plan that will respect such upper boundary. This process can be performed in an iterative fashion.

If the implant is selected first, the surgical plan can be adapted to respect a given implant thickness, e.g., a minimum implant thickness to withstand fatigue fracture of the implant, or a maximum implant thickness to leave sufficient underlying bone stock, e.g., for future revision surgery. The implant thickness can include a fixed minimum or can be constant, while the implant includes one or more patient-adapted features, e.g., an adaptation to an ML width of a femoral condyle. In some embodiments, the implant may include a patient-adapted joint-facing surface (e.g., external surface) and a bone-facing surface (e.g., internal surface) that is derived from minimum and/or maximum implant thickness requirements and the patient-adapted external surface. Accordingly, in some embodiments, the shape of the implant's bone-facing surface may follow the patient-adapted joint-facing surface (in whole or in part), offset by the thickness (e.g., minimal thickness) of the implant. Therefore, in some embodiments, the implant's joint-facing surface may, at least in part, be irregular and/or organically shaped. In some embodiments, the joint-facing surface may include one or more irregular and/or organically shaped portions, as well as one or more planar portions. A robot may facilitate the implantation of such a minimal thickness implant by modifying the implantation site (e.g., a femoral condyle), for example, by burring.

The surgical plan and the site and location of the intended implantation as well as the selection, adaptation or design of the implant can also be adapted for different biomechanical conditions.

A robot can also allow for the implantation of an implant with an irregular or organically shaped bone-facing surface. While it may be difficult to precisely prepare the implantation site for an implant with an irregular or organically shaped bone-facing surface manually or using mechanical tools alone, this can be greatly facilitated by using a robot. The shape information for the bone-facing surface is included in the surgical plan, which is electronically transferred to and executed by the robot. For example, the robot could resect the bone at the implant site to substantially match the bone-facing surface of the implant based on the shape information.

Similarly, an implant with an organically shaped bone-facing surface can be an implant that is fitted to the specific patient's bone surface as an inlay implant. Images of the patient's joint can be used to derive information on the 3-dimensional shape of the patient's bone, which is used to determine the shape of the internal implant surface. The internal implant surface can be sunk into the patient's bone by a uniform distance, e.g., 2 mm or 3 mm. Alternatively, the distance by which the implant is laid into the bone can be variable, for example to compensate for deformation of the patient's bone. Other areas of the bone-facing surface can also be laid on top of the bone, i.e. they conform to the bone surface.

In another embodiment of this invention, the bone-facing surface of the implant can be a combination of organically shaped areas, regularly shaped areas (e.g., spherical or elliptical), and/or flat areas.

### Patient-Specific Registration Features

In various embodiments, including those of FIGS. 195 and 196, the surgical system can incorporate one or more patient-specific or patient-engineered registration features 19780, 19880 that desirably align the robotic system with known features of the patient's anatomy.

In certain embodiments, the robotic surgical system can include registration features that incorporate one or more patient-specific surfaces (i.e., inner or outer surfaces) that correspond to a portion of the patient's corresponding biological structure (e.g., bone or cartilage surface). In this way, the conforming surfaces fit optimally on the particular patient's bone and thereby provides a secure and unique reference point from which the robotic system can align itself. The registration feature can include confirming features such as detents and/or pressure sensitive structures to ensure adequate and correct contact with the underlying anatomical features. The registration features may form part of a surgical tool or tools for preparing the anatomical surface, including routers, cutting saws and/or drills. In certain embodiments, the registration feature may be used in conjunction with a surgical tool to prepare an anatomical surface, and then a subsequent registration feature and tool combination can incorporate a surface that corresponds to the prepared anatomical surface for subsequent surgical placement and anatomical surface preparation.

In various embodiments, the surgical plans and use of registration features can be developed and executed for a patient adapted or patient specific implant. The patient adapted or patient specific implant can be selected, adapted or designed using, for example, one or more of the features described in Table 2.

The surgical plans can be designed for implants with any combination of patient specific, patient engineered or standard features as described in Table 3.

The surgical plan can be designed or implemented for single implant components or multi-component systems including, for example, hybrid system components and combinations.

The surgical plan can be designed or implemented using any single or combination of patient specific measurements, as shown for example in Table 1; the same one or more patient specific measurements can also be used for designing, adapting or selecting implant shapes or features.

In various embodiments, the robotic surgical system can be utilized to perform various surgical operations, either autonomously or under direct control/supervision of the surgeon. If desired, a “dead-man switch” or other direct control mechanism could be used by the surgeon (such as, for example, a floor switch that the surgeon steps on to allow the robot to perform operation steps, but that immediately stops all robotic actions when the floor switch is released). The preparation of the surgical site can include burring, drilling, sawing (e.g., with a straight blade, a curved blade, blades with variable radii), mechanical abrasion, laser abrasion, and/or any other technique known in the art or developed in the future to prepare a surgical site. If desired, the robotic surgical system could comprise a registration feature that, when placed against an appropriate anatomical site (i.e., against a femoral head) and the system is activated, performs all surgical cutting and shaping steps to prepare the anatomical site for the intended implant. Once surgical preparation was complete, the system could indicate the readiness of the site for the implant. A second registration feature could be similarly suited for robotic preparation of the tibial surfaces.

If desired, the robotic system could include components or other features that place the implant into its intended implantation site and/or inject bone cement or other adhesive to secure the implant. The system could also secure the implant against the anatomical site for the amount of time necessary to allow the adhesive to secure the implant (i.e., for the curing time).

Implant selection and/or design data, with optional user-defined parameters, may be inputted or transferred by a user and/or by electronic transfer into a software-directed computer system that performs a series of operations to transform the data and optional parameters into one or more implant manufacturing specifications. Implant design data or implant manufacturing data, optionally with user-defined parameters, may be inputted or transferred by a user and/or by electronic transfer into a software-directed computer system that directs one or more manufacturing instruments to produce one or more implant components from a starting material, such as a raw material or starting blank material. Implant design data, implant manufacturing data, or implant data, optionally with user-defined parameters, may be inputted or transferred by a user and/or by electronic transfer into a software-directed computer system that performs a series of operations to transform the data and optional parameters into one or more surgical procedure specifications or instructions. Implant design data, implant manufacturing data, implant data, or surgical procedure data, optionally with user-defined parameters, may be inputted or transferred by a user and/or by electronic transfer into a software-directed computer system that directs one or more automated surgical instruments, for example, a robot, to perform one or more surgical steps. In certain embodiments, one or more of these actions can be performed as steps in a single process by one or more software-directed computer systems.

In certain embodiments, the implant component includes one or more bone cuts on its bone-facing surface. Features of these bone cuts, and optionally features of corresponding resection cuts, can be optimized by a computer system based on patient-specific data. For example, the bone cut number and one or more bone cut planes, angles, or depths, as well as the corresponding resection cut number and one or more resection cut planes, angles, or depths, can be optimized, for example, to preserve the maximum amount of bone for each individual patient based on a series of two-dimensional images or a three-dimensional representation of the articular anatomy and geometry and/or on a target limb alignment and/or deformity correction. Optionally, one or more of the bone cut features and/or resection cut features can be adjusted by the operator.

The computer system also can construct the implant surfaces. Surfaces may be composed of different elements. In certain embodiments, elements of the surfaces can conform to the patient's anatomy. In these situations, the computer system can build a surface using the patient's anatomical model, for example by constructing a surface that is identical with or mostly parallel to the patient's anatomical surface. In certain embodiments, the computer system can use geometric elements such as arcs or planes to construct a surface. Transitions between surfaces can be smoothed using tapers or fillets. Additionally, the computer system may take into account constraints such as a minimum or maximum threshold thickness or length or curvature of parts or features of the implant component when constructing the surfaces.

In another embodiment, the computer system can automatically or semi-automatically add other features to the implant design. For example, the computer system can add pegs or anchors or other attachment mechanisms. The system can place the features using anatomical landmarks. Constraints can be used to restrict the placement of the features. Examples of constraints for placement of pegs are the distance between pegs and from the pegs to the edge of the implant, the height of the pegs that results from their position on the implant, and forcing the pegs to be located on the center line. Optionally, the system can allow the user to fine-tune the peg placement, with or without enforcing the constraints.

### Hybrid Systems

The implants and implant systems described herein include any number of patient-adapted implant components and any number of non-patient-adapted implant components. In certain embodiments, the implants and implant systems described herein can include a combination of implant components, such as a traditional unicompartmental device with a patient-specific bicompartmental device or a combination of a patient-specific unicompartmental device with standard bicompartmental device. Such implant combinations allow for a flexible design of an implant or implant system that includes both standard and patient-specific features and components. This flexibility and level of patient-specificity allows for various engineered optimizations, such as retention of alignments, maximization of bone preservation, and/or restoration of normal or near-normal patient kinematics. In certain embodiments, an implant component is designed and installed as one or more pieces.

Embodiments described herein can be applied to partial or total joint replacement systems. Bone cuts or changes to an implant component dimension described herein can be applied to a portion of the dimension, or to the entire dimension.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method for treating a joint of a patient, comprising:
providing an implant component having a patient-adapted curvature in a first plane and a standard curvature in a second plane; and
utilizing an automated system during at least a portion of a procedure to implant the component into the joint,
wherein the patient-adapted curvature is derived from the patient's information.

2. The method of claim 1, wherein the first plane is a sagittal plane and the second plane is a coronal plane.

3. The method of claim 1, wherein the automated system includes a robot.

4. The method of claim 1, further comprising utilizing a guide tool during at least a portion of the procedure to implant the component into the joint.

5. A system for treating a joint of a patient, comprising:
an implant component having a patient-adapted feature and a standard feature; and
an automated system configured to assist with implanting the component into the joint.

6. The system of claim 5, wherein the patient-adapted feature includes a patient-adapted curvature in a first plane.

7. The system of claim 5, wherein the standard feature includes a standard curvature in a second plane.

8. The system of claim 5, further comprising a guide tool configured to guide resection of bone at the implantation site.

9. A method for treating a joint of a patient, comprising:
receiving information associated with at least a portion of the joint;
providing an implant component having patient-adapted features;
deriving a surgical plan based at least in part on the information and on features of the implant component; and
implanting the implant component into the joint according to the surgical plan.

10. The method of claim 9, wherein the implant component has a joint-facing surface that includes a patient-adapted external curvature in at least one plane.

11. The method of claim 10, wherein the shape of the implant component's bone-facing surface includes at least a portion derived from the implant component's joint-facing surface and a predetermined minimum thickness of the implant.

12. The method of claim 11, further comprising utilizing an automated system to prepare an implantation site of the joint based on the shape of the implant component's bone-facing surface.

13. The method of claim 12, where the step of utilizing an automated system to prepare an implantation site includes resecting portions of bone at the implantation site to match the shape of the implant component's bone-facing surface.

14. The method of claim 11, wherein the shape of the implant component's bone-facing surface further includes a planar portion.

15. The method of claim 9, wherein the features of the implant component comprise at least one of a minimum implant thickness, a maximum implant thickness and a patient-adapted external curvature.

16. An implant component for treating a joint of a patient, comprising:
a joint-facing surface having a shape, which includes a patient-adapted curvature in a first plane and a standard curvature in a second plane;
a bone-facing surface; and
a minimum thickness, which separates the joint-facing surface from the bone-facing surface and which is derived from information including data associated with the patient,
wherein the bone-facing surface has a shape, which includes at least a portion substantially derived from the implant component's joint-facing surface shape and the minimum thickness.

17. The implant component of 16, wherein the shape of the bone-facing surface further includes a substantially planar portion.

18. The implant component of claim 16, wherein the shape of the joint-facing surface is derived from the patient's information.

19. (canceled)
